(19) United States
(12) Patent Application Publication
(10) Pub. No.: US 2024/0327385 A1
(43) Pub. Date: Oct. 3, 2024

(54) SUBSTITUTED PIPERIDINE COMPOUNDS AS RENIN INHIBITORS

(71) Applicant: Guibai LIANG, Scotch Plains, NJ (US)

(72) Inventors: Jinyou XU, Scotch Plains, NJ (US); Guibai LIANG, Scotch Plains, NJ (US); Siyuan LE, Newtown Square, PA (US)

(21) Appl. No.: 18/598,315

(22) Filed: Mar. 7, 2024

Related U.S. Application Data
(60) Provisional application No. 63/489,882, filed on Mar. 13, 2023.

(51) Int. Cl. C07D 401/14; A61K 31/444; A61K 31/5377; A61P 9/12; C07D 401/12; C07D 405/14; C07D 413/14

(52) U.S. Cl. CPC C07D 401/14; A61K 31/444; A61K 31/5377; A61P 9/12; C07D 401/12; C07D 405/14; C07D 413/14

(57) ABSTRACT

Compounds having the formula I or II, pharmaceutical compositions containing the same, and their uses as renin inhibitors.

This application claims priority to U.S. provisional application No. 63/489,882 filed on Mar. 13, 2023.

### INCORPORATION BY REFERENCE

All documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

Citation or identification of any document in this application is not an admission that such document is available as prior art to the present disclosure.

### FIELD OF THE INVENTION

This application relates to novel 3, 5-substituted piperidine compounds and their derivatives, which have superior renin inhibitory activity over prior art molecules. The compounds, and their derivatives of the disclosure are useful for the treatment of diseases associated with renin activity, particularly diseases associated with excessive renin activity.

### BACKGROUND OF THE INVENTION

The renin-angiotensin-aldosterone system (RAAS) plays a critical role in the regulation of cardiovascular and renal physiology. A cascade of events of this system begins with conversion of angiotensinogen to angiotensin I by plasma renin, a circulating aspartic protease synthesized in juxtaglomerular epithelioid cells and released in granules in a controlled manner, then to angiotensin II catalyzed by the angiotensin-converting enzyme (ACE) expressed on the surface of vascular endothelial cells (Castrop H et al., (2010) Physiol. Rev. 90(2): 607-673). Angiotensin II may bind the angiotensin type-1 receptor to induce increased sodium retention, vasoconstriction, and aldosterone secretion (Ames M K et al., (2019) J Vet Intern Med. 33(2): 363-382). While aldosterone, the terminal hormone of the RAAS, is the key regulator of sodium, potassium and body fluid balance, its release may trigger sodium reabsorption by renal tubules, followed by water reabsorption into the blood and extracellular fluid volume increase in the body (Seelinger E. et al., (2005) Clin Exp Pharmacol Physiol. 32 (5-6): 394-399).

The renin-angiotensin-aldosterone system is stimulated under normal conditions in response to threats that compromise blood pressure stability, and its chronic activation may cause pathologic remodeling and dysfunction in cardiovascular and renal tissues, leading to cardiovascular disease, hypertension, diabetic kidney disease, heart failure, or other tissue/organ pathological conditions (Ames M K et al., (2019) supra).

RAAS suppression is a key strategy to relieve these conditions. Inhibitors to RRAS or its components include angiotensin-converting enzyme (ACE) inhibitors, angiotensin II receptor blockers (ARBs), and renin inhibitors. Among them, the renin inhibitors that block the first and rate-limiting step of RAAS activation attracted much attention since 1970s. However, it took the scientists about three decades to find the first and only potent one with acceptable oral bioavailability, aliskiren, which is approved in 2007 for clinical treatment of hypertension (Jensen C. et al., (2008) Nat Rev Drug Discov 7(5): 399-410). Angioedema, high blood potassium level, diarrhea, headache, dizziness and cough were observed as adverse effects in patients taking aliskiren.

More renin inhibitors having improved oral bioavailability, specificity, and efficacy with less severe side effects are needed.

### SUMMARY OF THE INVENTION

The present inventors have invented 3, 5-substituted piperidine compounds that have superior inhibitory effects on renin activity and are useful for prophylaxis or treatment of diseases associated with renin activity, e.g., excessive renin activity.

In one aspect, the present application relates to compounds of Formula I, or their respective geometric isomers, and pharmaecutically acceptable isotopic isomers, salts, prodrugs and solvates thereof,

wherein R₁ and R₂ may be independently a hydrogen atom, a deuterium atom, a halogen atom, a hydroxyl group, a carboxyl group, a cyano group, an optionally substituted C₁₋₆ alkyl group, an optionally substituted C₂₋₆ alkenyl group, an optionally substituted C₂₋₆ alkynyl group, an optionally substituted C₁₋₃ alkylthio group, an optionally substituted C₁₋₃ alkylsulfinyl group, an optionally substituted C₁₋₃ alkylsulfonyl group, an optionally substituted C₁₋₆ alkoxy group, an optionally substituted C₁₋₆ alkoxyC₁₋₆ alkyl group, an optionally substituted C₁₋₃ alkyl C₁₋₆ alkoxy group, an optionally substituted C₃₋₆ cycloalkyl group, an optionally substituted C₁₋₃ alkyl C₃₋₆ cycloalkyl group, an optionally substituted amino group, an optionally substituted C₁₋₃ alkyl amino group, an optionally substituted mercapto group, an optionally substituted aminocarbonyl group, an optionally substituted carbonyl group, an optionally substituted C₁₋₆ alkyl carbonyl group, or an optionally substituted C₁₋₃ alkoxycarbonyl group,
n may be 0, 1, 2 or 3,
X may be a methylene group, an oxygen atom, an amine group, a sulfinyl group or a sulfonyl group,
Ring A may be an optionally substituted 5- or 6-membered heterocycle that contains one or more N, O, S, SO and SO₂,
R₃ may be a hydrogen atom, a deuterium atom, a halogen atom, a hydroxyl group, a carboxyl group, an optionally substituted C₁₋₆ alkyl group, an optionally substituted C₃₋₆ cycloalkyl group, an optionally substituted C₂₋₆ alkenyl group, an optionally substituted C₂₋₆ alkynyl group, an optionally substituted C₁₋₆ alkoxy group, an optionally substituted amino group, an optionally substituted mercapto group, an optionally substituted aminocarbonyl group, an optionally substituted C₁₋₆ alkylcarbonyl group, an optionally substituted C₁₋₆ alkoxyC₁₋₆ alkyl group, or an optionally substituted C₁₋₃ alkoxycarbonyl group,
R₄ may be a hydrogen atom, a halogen atom, a deuterium atom, a hydroxyl group, a cyano group, an optionally substituted amino group, an optionally substituted C₁₋₆ alkyl group (e.g., a hydroxyl C₁₋₆ alkyl group), an optionally substituted C₁₋₆ haloalkyl group, an optionally substituted C₂₋₆ alkenyl group, an optionally substituted C₂₋₆ alkynyl group, an optionally substituted mercapto group, an optionally substituted C₁₋₆ alkylsulfinyl group, an optionally substituted C₁₋₆alkylsulfonyl group, an optionally substituted C₁₋₆ alkoxy group, an optionally substituted C₁₋₆ haloalkoxy group, an optionally substituted C₃₋₆ cycloalkyl group, an optionally substituted aminocarbonyl group, an optionally substituted C₁₋₆ alkylcarbonyl group, an optionally substituted C₁₋₆ haloalkylcarbonyl group, an optionally substituted C₁₋₃ alkoxycarbonyl group, an optionally substituted C₁₋₃ haloalkoxycarbonyl group, an optionally substituted C₁₋₆ alkoxyC₁₋₆ alkyl group, an optionally substituted C₁₋₆ haloalkoxyC₁₋₆ alkyl group, a 4- to 6-membered heterocycle substituted formyl group wherein the 4 to 6-membered heterocycle may contain one or more N, O, S, SO, and SO₂ and may be optionally substituted, an optionally substituted 3 to 6-membered cyclic hydrocarbon group, an optionally substituted 5- or 6-membered heterocycle, saturated or unsaturated, wherein the heterocycle may contain one or more N, O, S, SO, and SO₂, or an optionally substituted amide group, and
R₅ may be a hydrogen atom, a deuterium atom, a halogen atom, a hydroxyl group, a cyano group, a mercapto group, an optionally substituted C₁₋₃ alkylsulfinyl group, an optionally substituted C₁₋₃ alkylsulfonyl group, an optionally substituted C₁₋₃ haloalkylsulfonyl group, an optionally substituted amino group, an optionally substituted C₁₋₆ alkyl group, an optionally substituted C₁₋₆ haloalkyl group, an optionally substituted C₂₋₆ alkenyl group, an optionally substituted C₂₋₆ alkynyl group, an optionally substituted C₁₋₆ alkoxy group, an optionally substituted C₁₋₆haloalkoxy group, an optionally substituted C₃₋₆ cycloalkyl group, an optionally substituted C₃₋₆ halocycloalkyl group, an optionally substituted C₁₋₃ alkylthio group, an optionally substituted C₁₋₃ alkylsulfinyl group, an optionally substituted C₁₋₃ haloalkylsulfinyl group, an optionally substituted C₃₋₄ cycloalkyl group, an optionally substituted aminocarbonyl group, an optionally substituted C₁₋₆ alkylcarbonyl group, an optionally substituted C₁₋₆ alkoxyC₁₋₆ alkyl group, an optionally substituted C₁₋₆ haloalkoxyC₁₋₆ alkyl group, an optionally substituted C₁₋₃ alkoxycarbonyl group, or an optionally substituted C₁₋₃ haloalkoxycarbonyl group.

R₁ and R₂ may attach to the same carbon atom or different carbon atoms.

When X is a methylene group, it may be substituted by R₁ and/or R₂,

R₁ and R₂ may independently have one or more substituents selected from the group consisting of a halogen atom, a hydroxyl group, a C₁₋₆ alkyl group, a C₁₋₆ haloalkyl group, a hydroxyl C₁₋₆ alkyl group, a C₁₋₆ alkoxy group, a C₁₋₆ haloalkoxy group, a C₁₋₃ alkoxyC₁₋₃ alkyl group, a C₁₋₃ haloalkoxyC₁₋₃ alkyl group, a C₁₋₆ alkylcarbonyl group, a C₁₋₆ haloalkylcarbonyl group, a C₁₋₆ alkyl amino group, and a C₁₋₆ haloalkyl amino group.

R₃ may have one or more substituents selected from the group consisting of a halogen atom, a hydroxyl group, a C₁₋₆ alkyl group, a C₁₋₆ haloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₃ alkyl group, a C₁₋₃ haloalkoxyC₁₋₃ alkyl group, a C₁₋₆ alkyl amino group, and a C₁₋₆ haloalkyl amino group.

Ring A may be an optionally substituted 5- or 6-membered heterocycle, partially unsaturated or unsaturated, containing one or more, e.g., one or two, N, O, S, SO and SO₂. In certain embodiments, ring A may be an optionally substituted 6-membered heterocycle, partially unsaturated or unsaturated, containing one or more, e.g., one, two or three atoms selected from N, O and S. In certain embodiments, ring A may be an optionally substituted 6-membered heterocycle, partially unsaturated or unsaturated, containing one or more, e.g., one, two or three N atoms. In certain embodiments, ring A may be an optionally substituted pyridine, an optionally substituted pyridazine, an optionally substituted pyrimidine, or an optionally substituted pyrazine. In certain embodiments, ring A may be an optionally substituted pyran, an optionally substituted thiopyran, an optionally substituted oxazine, an optionally substituted thiazine, an optionally substituted dioxin, an optionally substituted dithiin, or an optionally substituted triazine.

Ring A may have one or more, e.g., one or two, substituents selected from the group consisting of a deuterium atom, a halogen atom, a cyano group, a hydroxyl group, a C₁₋₆ alkyl group, a C₁₋₆ haloalkyl group, a C₂₋₆ alkenyl group, a C₂₋₆ haloalkenyl group, a C₂₋₆ alkynyl group, a C₂₋₆ haloalkynyl group, a C₃₋₆ cycloalkyl group, a C₁₋₆ alkylsulfinyl group, a C₁₋₆ haloalkylsulfinyl group, a C₁₋₆ alkylsulfonyl group, a C₁₋₆ haloalkylsulfonyl group, a C₁₋₆ alkoxy group, a C₁₋₆ haloalkoxy group, an amino group, a C₁₋₆ alkylcarbonyl group, a C₁₋₆ haloalkylcarbonyl group, a C₁₋₃ alkoxycarbonyl group, a C₁₋₃ haloalkoxycarbonyl group, a C₁₋₆ alkoxyC₁₋₆ alkyl group, a C₁₋₆ haloalkoxyC₁₋₆ alkyl group, or an optionally substituted 5 or 6-membered heterocycle containing one or more N, O, S, SO, and SO₂, wherein the substituents may be each further contain one or more, e.g., one or two, substituents selected from the group consisting of a halogen atom, a hydroxyl group, a C₁₋₆ alkyl group, a C₁₋₆ haloalkyl group, a C₃₋₆ cycloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₃ alkyl group, a C₁₋₃ haloalkoxyC₁₋₃ alkyl group, a C₁₋₆ alkyl amino group and a C₁₋₆ haloalkyl amino group. In certain embodiments, Ring A may have a C₁₋₆ alkyl group, as the substituent, which may be unsubstituted or substituted with one or more selected from the group consisting of a halogen atom, a C₁₋₃ alkyl group and a C₁₋₃ haloalkyl group. In certain embodiments, Ring A may have a C₃₋₆ cycloalkyl group, as the substituent, which may be unsubstituted or substituted with one or more selected from the group consisting of a halogen atom, a C₁₋₃ alkyl group and a C₁₋₃ haloalkyl group. In certain embodiments, Ring A may have an amino group, as the substituent, which may be unsubstituted or substituted with one or more selected from the group consisting of a C₁₋₃ alkyl group and a C₁₋₃ haloalkyl group.

In certain embodiments, ring A may be any one of following:

wherein R₁′, R₂′, and R₃′ may be independently a hydrogen atom, a deuterium atom, a halogen atom, a cyano group, a hydroxyl group, an optionally substituted C₁₋₆ alkyl group, an optionally substituted C₁₋₆ haloalkyl group, an optionally substituted C₂₋₆ alkenyl group, an optionally substituted C₂₋₆ haloalkenyl group, an optionally substituted C₂₋₆ alkynyl group, an optionally substituted C₂₋₆ haloalkynyl group, an optionally substituted C₃₋₆ cycloalkyl group, an optionally substituted C₁₋₆ alkylsulfinyl group, an optionally substituted C₁₋₆ haloalkylsulfinyl group, an optionally substituted C₁₋₆ alkylsulfonyl group, an optionally substituted C₁₋₆ haloalkylsulfonyl group, an optionally substituted C₁₋₆ alkoxy group, an optionally substituted C₁₋₆ haloalkoxy group, an optionally substituted amino group, an optionally substituted C₁₋₆ alkylcarbonyl group, an optionally substituted C₁₋₆ haloalkylcarbonyl group, an optionally substituted C₁₋₃ alkoxycarbonyl group, an optionally substituted C₁₋₃ haloalkoxycarbonyl group, an optionally substituted C₁₋₆ alkoxyC₁₋₆ alkyl group, an optionally substituted C₁₋₆ haloalkoxyC₁₋₆ alkyl group, or an optionally substituted 5 or 6-membered heterocycle containing one or more N, O, S, SO, and SO₂.

R₁′, R₂′, and R₃′ may independently have one or more substituents selected from the group consisting of a halogen atom, a hydroxyl group, a C₁₋₆ alkyl group, a C₁₋₆ haloalkyl group, a C₃₋₆ cycloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₃ alkyl group, a C₁₋₃ haloalkoxyC₁₋₃ alkyl group, a C₁₋₆ alkyl amino group and a C₁₋₆ haloalkyl amino group. In certain embodiments, R₁′, R₂′, and R₃′ may be independently a C₁₋₆ alkyl group which may be unsubstituted or substituted with one or more selected from the group consisting of a halogen atom, a C₁₋₃ alkyl group and a C₁₋₃ haloalkyl group. In certain embodiments, R₁′, R₂′, and R₃′ may be independently a C₃₋₆ cycloalkyl group which may be unsubstituted or substituted with one or more selected from the group consisting of a halogen atom, a C₁₋₃ alkyl group and a C₁₋₃ haloalkyl group. In certain embodiments, R₁′, R₂′, and R₃′ may be independently an amino group which may be unsubstituted or substituted with one or more selected from the group consisting of a halogen atom, a C₁₋₃ alkyl group and a C₁₋₃ haloalkyl group.

R₄ may have one or more substituents selected from the group consisting of a deuterium, a halogen atom, a hydroxyl group, a cyano group, an amino group, a halo-substituted amino group, a C₁₋₆ alkyl group, a C₁₋₆haloalkyl group, a C₃₋₆ cycloalkyl group, a C₃₋₆halocycloalkyl group, a C₁₋₃ alkyl C₃₋₆ cycloalkyl group, a C₁₋₃ haloalkyl C₃₋₆ cycloalkyl group, a hydroxyl C₁₋₆ alkyl group, a hydroxyl C₃₋₆cycloalkyl group, a C₁₋₆ alkylcarbonyl group, a C₁₋₆ alkoxy group, a C₁₋₆ haloalkoxy group, a C₁₋₆ haloalkylcarbonyl group, a C₃₋₆ halocycloalkylcarbonyl group, a C₁₋₃ alkoxyC₁₋₃ alkyl group, a C₁₋₆ alkyl amino group, a C₁₋₆ haloalkyl amino group, a C₁₋₃ haloalkoxyC₁₋₃ alkyl group, and a 3- or 6-membered heterocycle which contains one or more selected from the group consisting of N, O, S, SO and SO₂.

R₅ may attach to any carbon atom in the piperidine ring.

R₅ may have one or more substituents selected from the group consisting of a deuterium atom, a halogen atom, a hydroxyl group, an amino group, a C₁₋₆ alkyl group, a C₁₋₆ haloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₆ alkyl group, a C₁₋₃ haloalkoxyC₁₋₆ alkyl group, a C₁₋₆ alkyl amino group, and a C₁₋₆ haloalkyl amino group.

In certain embodiments, X may be a methylene group, an oxygen atom, or an amine group. When X is a methylene group, it may be unsubstituted or substituted by R₁ and/or R₂.

In certain embodiments, n may be 0 or 1.

In certain embodiments, R₁ and R₂ may be independently a hydrogen atom, a halogen atom, a hydroxyl group, a C₁₋₆ alkyl group, a C₁₋₆ haloalkyl group, a C₃₋₆ cycloalkyl group, a C₁₋₆ alkoxy group, an amino group, an aminocarbonyl group, a C₁₋₆ alkylcarbonyl group, a C₁₋₆ haloalkylcarbonyl group, a C₁₋₆ alkoxyC₁₋₆ alkyl group, a C₁₋₆ haloalkoxyC₁₋₆ alkyl group, a C₁₋₃ alkoxycarbonyl group or a C₁₋₃ halolkoxycarbonyl group. In certain embodiments, these groups may each contain one or more, e.g., one or two, substituents selected from the group consisting of a halogen atom, a hydroxyl group, a C₁₋₆ alkyl group, a C₁₋₆ haloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₃ alkyl group, a C₁₋₃ haloalkoxyC₁₋₃ alkyl group, a C₁₋₆ alkylamino group and a C₁₋₆ haloalkylamino group. In certain embodiments, R₁ and R₂ may be independently a hydrogen atom, a halogen atom, a hydroxyl group, a C₁₋₆ alkyl group, a C₁₋₆ haloalkyl group, a C₃₋₆ cycloalkyl group (which may be unsubstituted or substituted with one or more selected from the group consisting of a halogen atom, a C₁₋₃ alkyl group and a C₁₋₃ haloalkyl group), a C₁₋₆ alkoxy group (which may be unsubstituted or substituted with one or more selected from the group consisting of a halogen atom, a C₁₋₃ alkyl group and a C₁₋₃ haloalkyl group), an amino group (which may be unsubstituted or substituted with one or more selected from the group consisting of a halogen atom, a C₁₋₃ alkyl group and a C₁₋₃ haloalkyl group), an aminocarbonyl group (which may be unsubstituted or substituted with one or more selected from the group consisting of a halogen atom, a C₁₋₃ alkyl group and a C₁₋₃ haloalkyl group), a C₁₋₆ alkylcarbonyl group, a C₁₋₆ haloalkylcarbonyl group, a C₁₋₆ alkoxyC₁₋₆ alkyl group, a C₁₋₆ haloalkoxyC₁₋₆ alkyl group, a C₁₋₃ alkoxycarbonyl group or a C₁₋₃ halolkoxycarbonyl group.

In certain embodiments, R₁′, R₂′, and R₃′ may independently be a hydrogen atom, a halogen group, a hydroxyl group, a C₁₋₆ alkyl group, a C₁₋₆ haloalkyl group, a C₃₋₆ cycloalkyl group, a C₁₋₆ alkoxy group, a C₁₋₆ haloalkoxy group, an amino group, a C₁₋₆ alkylcarbonyl group, a C₁₋₆ haloalkylcarbonyl group, a C₁₋₃ alkoxycarbonyl group, a C₁₋₃ haloalkoxycarbonyl group, a C₁₋₆ alkoxyC₁₋₆ alkyl group, a C₁₋₆ haloalkoxyC₁₋₆ alkyl group, or a 5- or 6-membered heterocycle containing one or more N, O, S, SO, and SO₂. In certain embodiments, these groups may each contain one or more, e.g., one or two, substituents selected from the group consisting of a halogen atom, a hydroxyl group, a C₁₋₆ alkyl group, a C₁₋₆ haloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₃ alkyl group, a C₁₋₃ haloalkoxyC₁₋₃ alkyl group, a C₁₋₆ alkyl amino group, and a C₁₋₆ haloalkyl amino group. In certain embodiments, R₁′, R₂′, and R₃′ may independently be a hydrogen atom, a halogen group, a hydroxyl group, a C₁₋₆ alkyl group, a C₁₋₆ haloalkyl group, a C₃₋₆ cycloalkyl group (which may be unsubstituted or substituted with one or more selected from the group consisting of a halogen atom, a C₁₋₃ alkyl group and a C₁₋₃ haloalkyl group), a C₁₋₆ alkoxy group, a C₁₋₆ haloalkoxy group, an amino group (which may be unsubstituted or substituted with one or more selected from the group consisting of a halogen atom, a C₁₋₃ alkyl group and a C₁₋₃ haloalkyl group), a C₁₋₆ alkylcarbonyl group, a C₁₋₆ haloalkylcarbonyl group, a C₁₋₃ alkoxycarbonyl group, a C₁₋₃ haloalkoxycarbonyl group, a C₁₋₆ alkoxyC₁₋₆ alkyl group, a C₁₋₆ haloalkoxyC₁₋₆ alkyl group, or a 5- or 6-membered heterocycle containing one or more N, O, S, SO, and SO₂.

In certain embodiments, R₃ may be a hydrogen atom, a deuterium atom, a halogen atom, a C₁₋₆ alkyl group, a C₃₋₆ cycloalkyl group, a C₁₋₆ alkoxy group, an amino group, an aminocarbonyl group, a C₁₋₆ alkylcarbonyl group, a C₁₋₆ alkoxyC₁₋₆ alkyl group, or a C₁₋₃ alkoxycarbonyl group. In certain embodiments, these groups may each contain one or more, e.g., one or two, substituents selected from the group consisting of a halogen atom, a hydroxyl group, a C₁₋₆ alkyl group, a hydroxyl C₁₋₆ alkyl group, a C₁₋₆ alkylcarbonyl group, a C₁₋₃ alkoxyC₁₋₃ alkyl group, and a C₁₋₆ alkyl amino group. In certain embodiments, R₃ may be a hydrogen atom, a deuterium atom, a halogen atom, a C₁₋₆ alkyl group (which may be unsubstituted or substituted with one or more selected from the group consisting of a halogen atom, a C₁₋₃ alkyl group and a C₁₋₃ haloalkyl group), a C₃₋₆ cycloalkyl group (which may be unsubstituted or substituted with one or more selected from the group consisting of a halogen atom, a C₁₋₃ alkyl group and a C₁₋₃ haloalkyl group), a C₁₋₆ alkoxy group (which may be unsubstituted or substituted with one or more selected from the group consisting of a halogen atom, a C₁₋₃ alkyl group and a C₁₋₃ haloalkyl group), an amino group (which may be unsubstituted or substituted with one or more selected from the group consisting of a halogen atom, a C₁₋₃ alkyl group and a C₁₋₃ haloalkyl group), an aminocarbonyl group, a C₁₋₆ alkylcarbonyl group, a C₁₋₆ alkoxyC₁₋₆ alkyl group, or a C₁₋₃ alkoxycarbonyl group.

In certain embodiments, R₄ may be a hydrogen atom, a deuterium atom, a halogen atom, a hydroxyl group, an amino group, an optionally substituted C₁₋₆ alkyl group, an optionally substituted C₃₋₆ cycloalkyl group, an optionally substituted C₁₋₆ alkoxy group, an optionally substituted aminocarbonyl group, an optionally substituted C₁₋₆ alkylcarbonyl group, an optionally substituted C₁₋₃ alkoxycarbonyl group, an optionally substituted C₁₋₆ alkoxyC₁₋₆ alkyl group, an optionally substituted 3- to 6-membered cyclic hydrocarbon group, a 5- or 6-membered saturated or partially unsaturated heterocycle wherein the heterocycle may contain one or more, e.g., one, two or three, N, O, S, SO and SO₂ and optionally substituted by e.g., an optionally substituted C₁₋₆ alkyl group, an optionally substituted C₃₋₆ cycloalkyl group, an optionally substituted 3 to 6-membered cyclic hydrocarbon group, an optionally substituted 3- or 6-membered heterocycle which contains one or more N, O, S, SO and SO₂, or a 4- to 6-membered heterocycle substituted formyl group wherein the heterocycle may contain one or more, e.g., one or two, N, O, S, SO, and SO₂ and optionally substituted by e.g., a halogen atom, an optionally substituted C₁₋₆ alkylcarbonyl group, an optionally substituted C₁₋₆ alkyl group.

In certain embodiments, these groups may each contain one or more, e.g., one or two, substituents selected from the group consisting of a halogen atom, a hydroxyl group, a C₁₋₆ alkyl group, a C₁₋₆ haloalkyl group, a C₃₋₆ cycloalkyl group, a C₃₋₆ halocycloalkyl group, a C₁₋₆ alkoxy group, a C₁₋₆ haloalkoxy group, a hydroxyl C₁₋₆ alkyl group, a hydroxyl C₃₋₆ cycloalkyl group, a C₁₋₆ alkylcarbonyl group, a C₁₋₆ haloalkylcarbonyl group, a C₃₋₆ halocycloalkylcarbonyl group, a C₁₋₃ alkoxyC₁₋₃ alkyl group, a C₁₋₆ alkyl amino group, a C₁₋₆ haloalkyl amino group, and a C₁₋₃ haloalkoxyC₁₋₃ alkyl group. In certain embodiment, R₄ may be a 3 to 6-membered cyclic hydrocarbon group which is unsubstituted or substituted with one or more selected from the group consisting of a halogen atom, a C₁₋₃ alkyl group and a C₁₋₃ haloalkyl group; a 5- or 6-membered unsaturated heterocycle which contains one or more selected from the group consisting of N, O, S, SO and SO₂ and is unsubstituted or substituted by one or more selected from the group consisting of a halogen atom, a hydroxyl group, a C₁₋₆ alkyl group, a C₁₋₆ haloalkyl group, a C₃₋₆ cycloalkyl group, a C₃₋₆ halocycloalkyl group, a hydroxyl C₁₋₆ alkyl group, a hydroxyl C₃₋₆ cycloalkyl group, a C₁₋₆ alkylcarbonyl group, a C₃₋₆ halocycloalkylcarbonyl group, a C₁₋₆ haloalkyl group, a C₃₋₆ halocycloalkyl group, a C₁₋₆ haloalkylcarbonyl group, a C₃₋₆ halocycloalkylcarbonyl group, a C₁₋₃ alkoxyC₁₋₃ alkyl group, a C₁₋₆ alkyl amino group, a 3- or 6-membered heterocycle which contains one or more selected from the group consisting of N, O, S, SO and SO₂ and is unsubstituted or substituted by a halogen atom, a C₁₋₃ alkyl group and a C₁₋₃ haloalkyl group, and a 3 to 6-membered cyclic hydrocarbon group which is unsubstituted or substituted with one or more selected from the group consisting of a halogen atom, a C₁₋₃ alkyl group and a C₁₋₃ haloalkyl group; a 4- to 6-membered heterocycle substituted formyl group wherein the heterocycle contains one or more selected from the group consisting of N, O, S, SO, and SO₂ and is unsubstituted or substituted by one or more selected from the group consisting of a halogen atom, a hydroxyl group, a C₁₋₆ alkyl group, a C₁₋₆ haloalkyl group, a C₃₋₆ cycloalkyl group, a C₃₋₆ halocycloalkyl group, a C₁₋₆ alkoxy group, a C₁₋₆ haloalkoxy group, a hydroxyl C₁₋₆ alkyl group, a hydroxyl C₃₋₆ cycloalkyl group, a C₁₋₆ alkylcarbonyl group, a C₃₋₆ halocycloalkylcarbonyl group, a C₁₋₆ haloalkyl group, a C₃₋₆ halocycloalkyl group, a C₁₋₆ haloalkylcarbonyl group, a C₃₋₆ halocycloalkylcarbonyl group, a C₁₋₃ alkoxyC₁₋₃ alkyl group, and a C₁₋₆ alkyl amino group; or a hydroxyl C₁₋₆ alkyl group.

In certain embodiments, R₅ may be a hydrogen atom, a halogen atom, a hydroxyl group, a C₁₋₆ alkoxy group, a C₁₋₆ haloalkoxy group, an amino group, a C₁₋₆ alkyl group, a C₁₋₆ haloalkyl group, a C₃₋₆ cycloalkyl group, a C₁₋₃ alkylsulfinyl group, a C₁₋₃ haloalkylsulfinyl group, a C₁₋₃ alkylsulfonyl group, a C₁₋₃ haloalkylsulfonyl group, an aminocarbonyl group, a C₁₋₆ alkylcarbonyl group, a C₁₋₆ alkoxyC₁₋₆ alkyl group, a C₁₋₆ haloalkoxyC₁₋₆ alkyl group, a C₁₋₃ alkoxycarbonyl group, or a C₁₋₃ haloalkoxycarbonyl group. In certain embodiments, these groups may each contain one or more, e.g., one or two, substituents selected from the group consisting of a halogen atom, a hydroxyl group, a C₁₋₆ alkyl group, a C₁₋₆ haloalkyl group, a hydroxyl C₁₋₆ alkyl group, a C₁₋₆ alkylcarbonyl group, a C₁₋₆ haloalkylcarbonyl group, a C₁₋₃ alkoxyC₁₋₆ alkyl group, a C₁₋₃ haloalkoxyC₁₋₆ alkyl group, and an amino group. In certain embodiments, R₅ may be a hydrogen atom, a halogen atom, a hydroxyl group, a C₁₋₆ alkoxy group, a C₁₋₆ haloalkoxy group, an amino group (which may be unsubstituted or substituted with one or more selected from the group consisting of a halogen atom, a C₁₋₃ alkyl group and a C₁₋₃ haloalkyl group), a C₁₋₆ alkyl group, a C₁₋₆ haloalkyl group, a C₃₋₆ cycloalkyl group (which may be unsubstituted or substituted with one or more selected from the group consisting of a halogen atom, a C₁₋₃ alkyl group and a C₁₋₃ haloalkyl group), a C₁₋₃ alkylsulfinyl group, a C₁₋₃ haloalkylsulfinyl group, a C₁₋₃ alkylsulfonyl group, a C₁₋₃ haloalkylsulfonyl group, an aminocarbonyl group (which may be unsubstituted or substituted with one or more selected from the group consisting of a halogen atom, a C₁₋₃ alkyl group and a C₁₋₃ haloalkyl group), a C₁₋₆ alkylcarbonyl group, a C₁₋₆ alkoxyC₁₋₆ alkyl group, a C₁₋₆ haloalkoxyC₁₋₆ alkyl group, a C₁₋₃ alkoxycarbonyl group, or a C₁₋₃ haloalkoxycarbonyl group.

In another aspect, the present application relates to compounds of Formula II, or their respective geometric isomers, and pharmaceutically acceptable isotopic isomers, salts, prodrugs and solvates thereof,

wherein R₁, R₂, R₃, R₅, X, n, ring A, R₁′, R₂′, and R₃′, if any, are defined as above, and wherein R₆ and R₇ may be independently a hydrogen atom, a deuterium atom, a halogen atom, a cyano group, a hydroxyl group, an optionally substituted C₁₋₃ alkyl group, an optionally substituted C₃₋₆ cycloalkyl group, or an optionally substituted C₁₋₃ alkoxy group.

R₆ and R₇ may independently have one or more, e.g., one or two, substituents selected from the group consisting of a halogen atom, a hydroxyl group, a C₁₋₃ alkyl group, a C₁₋₃ haloalkyl group, a hydroxyl C₁₋₃ alkyl group, a C₁₋₃ alkoxy group, a C₁₋₃ haloalkoxy group, and an amino group.

In certain embodiments, R₆ and R₇ may be independently a hydrogen atom, a deuterium atom, a halogen atom, a cyano group, a hydroxyl group, a C₁₋₃ alkyl group, a C₁₋₃ haloalkyl group, a C₃₋₆ cycloalkyl group, a C₃₋₆ halocycloalkyl group, a C₁₋₃ alkoxy group, or a C₁₋₃ haloalkoxy group.

R₆ and R₇ may attach to the same atom in the ring.

In certain embodiments, the compounds of the disclosure may be selected from the group consisting of:
(3S)—[N-(2-methylpropyl), N-(5-cyclopropyl-3-cyclopropylamino)pyridine-2-carbonyl]amino-(5R)-[(3S)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(3-fluoroazetidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(3,3-difluoropyrrolidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(4,4-difluoropiperidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(4-trifluoromethoxypiperidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-[(3R)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-[(3S)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(2,2-dimethylmorpholine-N-carbonyl)-piperidine,
(3S)—{N-(2-methylpropyl), N-{2-tert-butyl-3-[3-(trans-methoxy)cyclobutyl]amino}pyrimidine-5-carbonyl}amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(morpholine-N-carbonyl)-piperidine,
(3S)—{N-(2-methylpropyl), N-{5-cyclopropyl-3-[3-(cis-methoxy)cyclobutyl]amino}pyridine-2-carbonyl}amino-(5R)-(morpholine-N-carbonyl)-piperidine,
(3S)—[N-(2-methylpropyl), N-(5-cyclopropyl-3-cyclobutylamino)pyridine-2-carbonyl]amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(5-methyl-1,3,4-oxadiazol-2-yl)-piperidine,
(3S)—{N-(2-methylpropyl), N-{5-cyclopropyl-3-[3-(trans-methoxy)cyclobutyl]amino}pyridine-2-carbonyl}amino-(5R)-(5-cyclopropyl-1,3,4-oxadiazol-2-yl)-piperidine,
(3S)—{N-(2-methylpropyl), N-{5-cyclopropyl-3-[3-(trans-methoxy)cyclobutyl]amino}pyridine-2-carbonyl}amino-(5R)-(3,3-diflurorocyclobutyl-1,3,4-oxadiazol-2-yl)-piperidine,
(3S)—[N-(2-methylpropyl), N-(5-cyclopropyl-3-cyclopropylamino)pyridine-2-carbonyl]amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-piperidine,
(3S)—{N-(2-methylpropyl), N-{5-cyclopropyl-3-[3-(trans-methoxy)cyclobutyl]amino}pyridine-2-carbonyl}amino-(5R)-[(3S)-ethylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(5-methyl-1,2,4-oxadiazol-3-yl)-piperidine,
(3S)—{N-(2-methylpropyl), N-{5-cyclopropyl-3-[3-(trans-methoxy)cyclobutyl]amino}pyridine-2-carbonyl}amino-(5R)-(3-methyl-1,2,4-oxadiazol-5-yl)-piperidine,
(3S)—[N-(2-methylpropyl), N-(5-cyclopropyl-3-(oxetan-3-ylamino)pyridine-2-carbonyl]amino-(5R)-(morpholine-N-carbonyl)-piperidine,
(3S)—{N-(2-methylpropyl), N-[3-(3-trans-methoxy)cyclobutyl]amino-pyridine-2-carbonyl}amino-(5R)-(5-cyclopropyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(5-cyclopropyl-1,3,4-oxadiazol-2-yl)-piperidine,
(3S)—{N-(2-methylpropyl), N-{6-cyclopropyl-4-[3-(trans-methoxy)cyclobutyl]amino}pyridazine-3-carbonyl}amino-(5R)-[(3S)-3-methylmorpholine-4-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-[5-(oxetan-3-yl)-1,3,4-oxadiazol-2-yl]-piperidine,
(3S)—{N-(2-methylpropyl), N-{5-cyclopropyl-3-[3-(trans-methoxy)cyclobutyl]amino}pyridine-2-carbonyl}amino-(5R)-hydroxymethyl-piperidine,
(3S)—{N-(2-methylpropyl), N-[5-cyclopropyl-3-(oxetan-3-yl)amino]pyridine-2-carbonyl}amino-piperidine,
(3S)—{N-(2-methylpropyl), N-[5-cyclopropyl-3-(oxetan-3-yl)amino]pyridine-2-carbonyl}amino-(5R)-(5-methyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(3-methyl-1,2,4-oxadiazol-5-yl)-piperidine,
(3S)—{N-(2-methylpropyl), N-{5-cyclopropyl-3-[3-(trans-methoxy)cyclobutyl]amino}pyridine-2-carbonyl}amino-(5R)-(1,2,4-oxadiazol-3-yl)-piperidine,
(3S)—{N-(2-methylpropyl), N-{5-cyclopropyl-3-[3-(trans-methoxy)cyclobutyl]amino}pyridine-2-carbonyl}amino-(5R)-(1,3,4-oxadiazol-2-yl)-piperidine,
(3S)—{N-(2-cyclopropylmethyl, N-{5-cyclopropyl-3-[3-(trans-methoxy)cyclobutyl]amino}-pyridine-2-carbonyl}amino-(5R)-(5-methyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(1,2,4-oxadiazol-3-yl)-piperidine,
(3S)—{N-(2-methylpropyl), N-[5-trifluoromethyl-3-(oxetan-3-yl)amino]pyridine-2-carbonyl}amino-(5R)-(morpholine-N-carbonyl)-piperidine,
(3S)—{N-cyclopropylmethyl, N-{5-cyclopropyl-3-[3-(trans-methoxy)cyclobutyl]amino}pyridine-2-carbonyl}amino-(5R)-(1,3,4-oxadiazol-2-yl)-piperidine,
(3S)—{N-(2-methylpropyl), N-{5-cyclopropyl-3-[3-(trans-methoxy-d3)cyclobutyl]amino}pyridine-2-carbonyl}amino-(5R)-(1,3,4-oxadiazol-2-yl)-piperidine, and
(3S)—{N-(2-methylpropyl), N-{2-cyclopropyl-3-[3-(trans-methoxy)cyclobutyl]amino}pyrimidine-5-carbonyl}amino-(5R)-[(3S)-methylmorpholine-N-carbonyl]-piperidine.

In certain embodiments, the compound is (3S)—[N-(2-methylpropyl), N-(5-cyclopropyl-3-cyclopropylamino)pyridine-2-carbonyl]amino-(5R)-[(3S)-methylmorpholine-N-carbonyl]-piperidine (Compound 21).

Also disclosed are the methods for preparing the compounds, the intermediate compounds, their respective geometric isomers, pharmaceutically acceptable isotopic isomers, salts, prodrugs and solvates thereof, of the disclosure, as well as pharmaceutical compositions comprising an effective amount of the compounds, their respective geometric isomers, pharmaceutically acceptable isotopic isomers, salts, prodrugs and solvates thereof, of the disclosure with a pharmaceutically acceptable carrier.

In a third aspect, the present application provides a method of using the compounds, their respective geometric isomers, pharmaceutically acceptable isotopic isomers, salts, prodrugs and solvates thereof, of the disclosure, as selective renin inhibitors, and for treatment of diseases associated with renin/RAAS activity such as hypertension, cardiovascular disease, diabetic kidney disease, and heart failure. The pharmaceutical composition of the disclosure may be used to inhibit renin activity, or the RAAS activity. The pharmaceutical composition of the disclosure may be used in preparation of a medicament for treating diseases associated with renin activity.

The present application provides a method for inhibiting renin activity in a subject in need thereof, comprising administering to the subject a pharmaceutical composition of the disclosure. The subject may be human. The subject may suffer from a disease caused by abnormal renin activity, such as hypertension, cardiovascular disease, diabetic kidney disease, and heart failure. In certain embodiments, the subject may suffer from hypertension and related complications.

The present application provides a method for inhibiting RAAS activity in a subject in need thereof, comprising administering to the subject a pharmaceutical composition of the disclosure. The subject may be human. The subject may suffer from a disease caused by abnormal RAAS activity, such as hypertension, cardiovascular disease, diabetic kidney disease, and heart failure. In certain embodiments, the subject may suffer from hypertension and related complications.

The present application provides a method for treating or alleviating a disease associated with renin/RAAS activity, e.g., abnormal renin/RAAS activity, in a subject in need thereof, comprising administering to the subject a pharmaceutical composition of the disclosure. The subject may be human. The disease may be hypertension, cardiovascular disease, diabetic kidney disease, or heart failure. In certain embodiments, the disease is hypertension.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the disclosure not to encompass within the disclosure any previously known product, process of making the product, or method of using the product such that Applicants reserve the right and hereby disclose a disclaimer of any previously known product, process, or method. It is further noted that the disclosure does not intend to encompass within the scope of the disclosure any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the disclosure to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the disclosure.

### DETAILED DESCRIPTION OF THE INVENTION

The present application provides novel 3, 5-substituted piperidine compounds and their derivatives which selectively and effectively inhibit renin activity. The compounds and their derivatives may be used to inhibit RAAS activity and treat or alleviate disease associated with abnormal renin/RAAS activity, including, but not limited to, hypertension, cardiovascular disease, diabetic kidney disease, and heart failure.

In a first embodiment, the compound of the disclosure has a structure of formula I or formula II illustrated below:

wherein R₁, R₂, X, n, Ring A, R₃, R₄, R₅, R₆, R₇, R₁′, R₂′, and R₃′, if any, are as defined above.

The exemplary compounds of formula I or formula II are set forth in Table 1 below.

```
TABLE 1
Exemplary compounds of the disclosure
Compound
  10
  11
  12
  13
  14
  15
  16
  17
  18
  19
  20
  21
  22
  23
  24
  25
  26
  27
  28
  29
  30
  31
  32
  33
  34
  35
  36
  37
  38
  39
  40
  41
  42
  43
  44
  45
  46
```

Various terms used throughout this specification and claims, unless otherwise limited in specific instances, either individually or as part of a larger group, have the following meanings.

The term “optionally substituted” means a group may be unsubstituted or substituted by one or more substituents.

The term “deuterium”, also called heavy hydrogen, refers to an isotope of hydrogen, the nucleus of which contains one proton and one neutron. Deuterium is abundant in oceans.

The term “halogen” or “halo” embraces six elements in Group 17 of the periodic table, including fluorine (F), chlorine (Cl), bromine (Br), iodine (I), astatine (At) and tennessine (Ts).

Preferred halogens in the present application are fluorine (F), chlorine (Cl), bromine (Br) and iodine (I), especially fluorine (F), chlorine (Cl), and bromine (Br).

The “carboxyl” refers to a functional group (—COOH) containing or consisting of a carbonyl group (C═O) with a hydroxyl group (O—H) attached to the same atom.

The term “cyano” herein refers to a functional group (—C—N) containing a carbon atom with a triple bond to a nitrogen atom.

The term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about eight, e.g., one to about six, one to about 3, carbon atoms. Examples of such radicals include methyl, ethyl, n-propyl, iso-propyl, n-butyl, iso-butyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about ten carbon atoms and more preferably about two to about eight, e.g., two to about six, two to five, two to four, carbon atoms. Examples of alkenyl radicals include ethenyl, allyl, propenyl, butenyl and 4-methylbutenyl. The terms “alkenyl”, and “lower alkenyl”, embrace radicals having “cis” and “trans” orientations, or alternatively, “E” and “Z” orientations.

The term “alkylthio” embraces radicals containing a linear or branched alkyl of one to about ten carbon atoms, attached to a divalent sulfur atom. More preferred alkylthio radicals are lower alkylthio radicals having alkyl radicals of one to about eight, e.g., one to six, one to three, carbon atoms. Examples of such lower alkylthio radicals are methylthio, ethylthio, propylthio, butylthio and hexylthio.

The term “alkynyl” embraces linear or branched radicals having at least one carbon-carbon triple bond of two to about twenty carbon atoms or, preferably, two to about twelve carbon atoms. More preferred alkynyl radicals are “lower alkynyl” radicals having two to about ten carbon atoms and more preferably about two to about eight, e.g., two to about six, two to five, two to four, carbon atoms. Examples of alkynyl radicals include propargyl, 1-propynyl, 2-propynyl, 1-butyne, 2-butynyl and 1-pentynyl.

The term “amide” refers to the —C(═O)N=radical.

The term “sulfinyl” refers to the —S(═O)— radical, while the term “alkylsulfinyl” embraces radicals containing a linear or branched alkyl radical, of one to about ten carbon atoms, attached to a divalent —S(═O)— radical. More preferred alkylsulfinyl radicals are lower alkylsulfinyl radicals having alkyl radicals of one to about eight, e.g., one to six, one to three, carbon atoms. Examples of such lower alkylsulfinyl radicals include methylsufinyl, ethylsufinyl, butylsufinyl and hexylsulfinyl.

The term “sulfonyl”, whether used alone or linked to other terms such as alkylsulfonyl, denotes the divalent radicals —SO₂—, while the term “alkylsulfonyl” embraces alkyl radicals attached to a sulfonyl radical, where alkyl is defined as above. More preferred alkylsulfonyl radicals are “lower alkylsulfonyl” radicals having one to about six, e.g., one to three, carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The term “alkoxy” embraces linear or branched oxy-containing radicals each having alkyl portions of one to about twenty carbon atoms or, preferably, one to about twelve carbon atoms. More preferred alkoxy radicals are “lower alkoxy” radicals having one to about ten carbon atoms and more preferably having one to about eight, e.g., one to six, one or three, carbon atoms. Examples of such radicals include methoxy, ethoxy, propoxy, butoxy and tert-butoxy.

The term “cycloalkyl” refers to cyclic saturated or unsaturated monovalent hydrocarbon radical of three to twelve carbon atoms. The term embraces saturated carbocyclic radicals having three to about twelve carbon atoms. The term “cycloalkyl” embraces saturated carbocyclic radicals having three to about twelve carbon atoms. More preferred cycloalkyl radicals are “lower cycloalkyl” radicals having three to about eight, e.g., three to six, three to four, carbon atoms. Examples of such radicals include cyclopropyl, cyclobutyl, cyclopentyl and cyclohexyl.

The term “hydroxyl cycloalkyl” embraces cycloalkyl radicals, as defined above, each substituted with one or more hydroxyl groups, such as hydroxyl C₃₋₆ cycloalkyl, and hydroxyl C₃₋₄ cycloalkyl.

The term “mercapto” refers to a functional group (—SH) containing a sulfur atom bonded to a hydrogen atom.

The term “carbonyl”, whether used alone or with other terms such as “alkylcarbonyl”, denotes a chemically organic functional group composed of a carbon atom double-bonded to an oxygen atom, i.e., —(C═O)—. The term “alkylcarbonyl” includes radicals having an alkyl radical, as defined above, attached to the carbon atom in a carbonyl radical. Examples of such radicals include substituted or unsubstituted methylcarbonyl, ethylcarbonyl, propylcarbonyl, butylcarbonyl, pentylcarbonyl and hexylcarbonyl. The term “aminocarbonyl” embraces radicals having an amino group, attached to the carbon atom in the carbonyl radical. The term “alkoxycarbonyl” means a radical containing an alkoxy radical, as defined above, attached to the carbon atom in a carbonyl radical. Examples of such “alkoxycarbonyl” radicals include substituted or unsubstituted methoxycarbonyl, ethoxycarbonyl, propoxycarbonyl, butoxycarbonyl and hexyloxycarbonyl.

The term “heterocycle” or “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Examples of unsaturated heterocycle radicals include pyridine, and diazine (including pyridazine, pyrimidine, and pyrazine). The partial unsaturated or unsaturated 6-membered heterocycle radicals also include pyran, thiopyran, oxazine, thiazine, dioxin, dithiin, and triazine. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “cyclic hydrocarbon group” refers to a saturated, partially unsaturated, or unsaturated carbon chain in a ring structure.

The term “formyl” refers to a functional group containing or consisting of a carbonyl group bonded to a hydrogen atom.

The term “hydroxyl alkyl” embraces alkyl radicals, as defined above, each substituted with one or more, e.g., one or two, hydroxyl radicals.

The term “alkyl amino” embraces amino radicals each substituted with one or two alkyl groups as defined above. Preferred alkylamino radicals have alkyl radicals having about one to about twenty carbon atoms or, preferably, one to about twelve, one to six, or one to three, carbon atoms. Examples of such radicals may be monosubstituted N-alkylamino or disubstituted N,N-alkylamino, such as N-methylamino, N-ethylamino, N,N-dimethylamino, N,N-diethylamino or the like.

The terms “haloalkyl”, “halocycloalkyl”, “haloalkoxy”, “halo-substituted amino”, “haloalkylcarbonyl”, “halocycloalkylcarbonyl” and the like respectively embrace alkyl, cycloalkyl, alkoxy, amino, alkylcarbonyl, cycloalkylcarbonyl and other radicals, as defined above, each substituted by one or more, e.g., one, two, three or four, halogens, as defined above.

The term “alkoxyalkyl” embraces alkyl radicals, as defined above, having one or more alkoxy radicals, as defined above, attached to the alkyl radical, that is, to form monoalkoxyalkyl and dialkoxyalkyl radicals.

The term “pyran” or “oxine” is a six-membered heterocyclic, non-aromatic ring, consisting of five carbon atoms and one oxygen atom with two double bonds, including 2H-pyran, and 4H-pyran.

The term “thiopyran” is a heterocyclic compound containing five carbon atoms and one sulfur atom with two double bonds, including 2H-thiopyran and 4H-thiopyran.

The term “oxazine” embraces heterocyclic radicals containing one oxygen and one nitrogen atom in a doubly unsaturated six-membered ring, including 1,2-oxazine, 1,3-oxazine, and 1,4-oxazine.

The term “thiazine” embraces radicals containing a ring of four carbon, one nitrogen and one sulfur atom, including 1,2-thiazine, 1,3-thiazine and 1,4-thiazine.

The term “dithiin” embraces radicals containing two sulfur and four carbon atoms, with two double bonds, including 1,2-dithiin and 1,4-dithiin.

The term “triazine” embraces radicals each with a six-membered benzene-like ring having three carbon and three nitrogen atoms, including 1,2,3-trizine, 1,2,4-triazine, and 1,3,5-triazine.

The term “aryl”, alone or in combination, generally means a carbocyclic aromatic system containing one, or more rings wherein such rings may be attached together in a pendent manner or may be fused. The term “aryl” embraces aromatic radicals such as phenyl, naphthyl, tetrahydronaphthyl, indane and biphenyl.

The term “substituted” refers to the replacement of one or more hydrogen radicals in a given structure with the radical of a specified substituent including, but not limited to, a deuterium atom, a halogen atom, a cyano group, a mercapto group, a hydroxyl group, a carboxyl group, an alkyl group, an alkenyl group, an alkynyl group, an alkoxy group, an cycloalkyl group, an amino group, an alkylthio group, a hydroxyl alkyl group, an alkoxy alkyl group, an alkylcarbonyl group, an aminocarbonyl group, an alkyl amino group, a cycloalkyl group, an alkylsulfinyl group, an alkylsulfonyl group, an amino group, an alkylcarbonyl group, an alkoxycarbonyl group, a heterocyclyl group, a hydroxyl cycloalkyl group, a haloalkyl group, a halocycloalkyl group, a haloalkylcarbonyl group, a halocycloalkylcarbonyl group, an alkylthioalkyl group, an alkylsulfonylalkyl group, an aminocarbonylcycloalkyl, an aminocarbonylheterocyclyl, an alkylaminocarbonyl, trifluoromethyl, an alkylaminoalkyl group, an aminoalkylamino group, an carboxyalkyl group, an alkoxycarbonylalkyl group, an aminocarbonylalkyl group, carboxylic acid, sulfonic acid, sulfonyl, phosphonic acid, pyridine, pyridazine, pyrimidine, pyrazine, pyran, thiopyran, oxazine, thiazine, dioxin, dithiin, and triazine. It is understood that the substituent may be further substituted.

Chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene”. Similarly, in circumstances in which divalent moieties are required and are stated as being “alkoxy”, “alkylamino”, “aryloxy”, “alkylthio”, “aryl”, “heteroaryl”, “alkyl”, “alkenyl”, “alkynyl”, or “cycloalkyl”, those skilled in the art will understand that the terms “alkoxy”, “alkylamino”, “aryloxy”, “alkylthio”, “aryl”, “heteroaryl”, “alkyl”, “alkenyl”, “alkynyl”, or “cycloalkyl” refer to the corresponding divalent moiety.

The term “compound” is defined herein to include pharmaceutically acceptable salts, solvates, hydrates, polymorphs, enantiomers, diastereoisomers, racemates and the like of the compounds having a formula as set forth herein.

The term “treatment” refers to any process, action, application, therapy, or the like, wherein a mammal, including a human being, is subject to medical aid with the object of improving the mammal's condition, directly or indirectly.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the disclosure, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The term “geometric isomer” embraces chemical species which contain the same type and quantity of atoms and bonds but have different spatial arrangements of the atoms.

The term “isotopic isomer”, also referred to as “isotopomer”, embraces isomers with isotopic atoms, having the same number of each isotope of each element but differing in their positions.

The term “pharmaceutically acceptable prodrugs” as used herein refers to those prodrugs of the compounds of the present disclosure which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present disclosure. “Prodrug”, as used herein means a compound which is convertible in vivo by metabolic means (e.g. by hydrolysis) to a compound of the disclosure. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development”, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this disclosure may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P.G.M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present disclosure is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

The compounds of the disclosure show inhibitory effects on human renin activity, with IC₅₀ less than 250.0 nM, less than 200.0 nM, less than 150.0 nM, less than 100.0 nM, less than 50.0 nM, less than 30.0 nM, less than 20.0 nM, less than 10.0 nM, less than 5.0 nM, or even less than 2.0 nM.

The pharmaceutical compositions of the present disclosure comprise a therapeutically effective amount of a compound of the present disclosure formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha-(α), beta-(β) and gamma-(γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this disclosure may be administered orally, parenterally, by inhalation spray, topically, rectally, nasally, buccally, vaginally or via an implanted reservoir, preferably by oral administration or administration by injection. The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

By a “therapeutically effective amount” of a compound of the disclosure is meant an amount of the compound which confers a therapeutic effect, e.g., reduction of abnormal renin/RAAS activity, on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg. Effective doses will also vary depending on route of administration, as well as the possibility of co-usage with other agents. It will be understood, however, that the total daily usage of the compounds and compositions of the present disclosure will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this disclosure administered to a human or other animal in single or in divided doses can be in amounts, for example, from 0.01 to 50 mg/kg body weight or more usually from 0.1 to 25 mg/kg body weight.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

A compound of the formula I, or a pharmaceutically-acceptable salt thereof, may be prepared by any process known to be applicable to the preparation of chemically-related compounds, including suitable processes for making certain intermediates. Necessary starting materials may be obtained by standard procedures of organic chemistry. The preparation of such starting materials is described within the accompanying non-limiting Examples. Alternatively necessary starting materials are obtainable by analogous procedures to those illustrated which are within the ordinary skill of a chemist.

The present disclosure further provides methods for the prevention or treatment of diseases or conditions involving abnormal renin activity or RAAS activity. In one embodiment, the disclosure further provides for the use of the pharmaceutical composition of the disclosure in the manufacture of a medicament for halting or decreasing diseases involving abnormal renin/RAAS activity. The disease may be hypertension, cardiovascular disease, diabetic kidney disease, or heart failure. In certain embodiments, the disease is hypertension. In one embodiment, the disclosure relates to a method of treating hypertension in a subject in need thereof comprising administering to said subject a therapeutically effective amount of a compound of the disclosure. In one embodiment, the disclosure relates to a method of inhibiting renin or RAAS activity in a subject in need thereof, comprising administering to the subject a pharmaceutical composition of the disclosure.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

The amount of the compound administered to the patient is less than an amount that would cause toxicity in the patient. In certain embodiments, the amount of the compound that is administered to the patient is less than the amount that causes a concentration of the compound in the patient's plasma to equal or exceed the toxic level of the compound. The optimal amount of the compound that should be administered to the patient in the practice of the present disclosure will depend on the particular compound used and the type of cancer being treated.

### EXAMPLES

The compounds described herein will be better understood in connection with the following representative synthetic schemes that illustrate the methods by which the compounds of the disclosure may be prepared, which are intended as an illustration only and not limiting of the scope of the disclosure. Various changes and modifications to the disclosed embodiments will be apparent to those skilled in the art and such changes and modifications including, without limitation, those relating to the chemical structures, substituents, derivatives, formulations and/or methods of the disclosure may be made without departing from the spirit of the disclosure and the scope of the appended claims.

### Example 1. Synthesis of intermediates

Synthesis of intermediate compound A

A solution of 3,5-dimethyl pyridine-3,5-dicarboxylate (34.1 g, 174.716 mmol, 1.0 eq) and PtO₂ (0.79 g, 3.494 mmol, 0.02 eq) in AcOH was stirred for 24 h at room temperature under hydrogen atmosphere. The resulting mixture was filtered; the filter cake was washed with AcOH (3×10 mL). The filtrate was concentrated under reduced pressure. This resulted in Compound 1 (35.1 g, crude) as a yellow oil. LCMS (ESI) [M+H]⁺: 202.

### Step B: Synthesis of dimethyl 1-tert-butoxlcarbonyl-piperidine 3,5-dicarboxylate (Compound 2)

A solution of Compound 1 (31 g, 154.059 mmol, 1.0 eq), Et₃N (77.95 g, 770.295 mmol, 5.0 eq) and Boc₂O (50.43 g, 231.089 mmol, 1.5 eq) in DCM was stirred for 15 h at room temperature. The residue was purified by silica gel column chromatography, eluted with PE/EA (2:1) to afford Compound 2 (32 g, 69%) as a yellow oil. LCMS (ESI) [M+H]⁺: 302.

### Step C: Synthesis of 1-tert-butoxycarbonyl-piperidine3,5-dicarboxylic acid (Compound 3)

A solution of Compound 2 (31 g, 102.874 mmol, 1.0 eq) and potassium carbonate (42.96 g, 308.622 mmol, 3.0 eq) in MeOH and H₂O was stirred for 15 h at 70° C. The mixture was acidified to pH 3 with HCl (aq.). The resulting mixture was extracted with EtOAc (3×200 mL). The combined organic layers were dried over anhydrous Na₂SO₄. After filtration, the filtrate was concentrated under reduced pressure. This resulted in Compound 3 (25 g, 89%) as a white solid. LCMS (ESI) [M+H]⁺: 274.

### Step D: Synthesis of 7-tert-butoxylcarbonyl-2,4-dioxo-3-oxa-7-azabicycle[3.3]nonane Compound 4)

A solution of Compound 3 (25 g, 91.480 mmol, 1.0 eq) in acetic anhydride (300 mL, 2938.612 mmol, 32.1 eq) was stirred for 15 h at 140° C. The resulting mixture was concentrated under vacuum. This resulted in Compound 4 (21.4 g, crude) as off-white solid. LCMS (ESI) [M+H]⁺: 256.

### Step E Synthesis of 1-(tert-butoxycarbonyl)-(5R)-(methoxycarbonyl)piperidine-(3S)-carboxylic acid (Compound 5)

To a solution of Compound 4 (10 g, 39.174 mmol) and (DHQD)₂AQN (3.34 g, 3.917 mmol) in a mixture of THF (250 mL) and Et₂O (700 mL) was added dropwise MeOH (16 mL, 395.18 mmol) in Et₂O (50 mL) at −78° C. The reaction mixture was stirred at −78° C. for 10 h and then warmed up to room temperature. The reaction mixture was acidified by 1N HCl (pH=3) and then extracted with EtOAc (100×3 mL). The organic layer was dried over Na₂SO₄, filtered, and concentrated in vacuo to give the mono-ester. The crude mono-ester was dissolved in EtOH (60 mL) at 80° C. To the mixture was added (R)-1-phenylethylamine (4.6 mL, 37.988 mmol) at 80° C., followed by stirring at room temperature overnight. The resulting crystals were filtered off and washed with acetonitrile to give the mono-ester. The intermediate salt in CH₂Cl₂ was then acidified by addition of 1N HCl (pH=3-4). The resulting mixture was extracted with CH₂Cl₂ (100×3 mL), and the combined organic extracts were dried over MgSO₄, filtered, and concentrated in vacuo. The residue was purified by reverse flash chromatography with the following conditions: column, silica gel; mobile phase, MeCN in water, 0% to 50% gradient in 20 min, UV 254 nm. This resulted in Compound 5 (2.2 g, 19.55%) as off-white solid. LCMS (ESI) [M+H]⁺: 288.

### Step F: Synthesis of methyl 1-tert-butoxylcarbonyl-(3S)-[(benzylogy)carbonyl]amino-piperidine-(5R)-carboxylate (Compound 6)

A solution of Compound 5 (300 mg, 1.044 mmol, 1.0 eq), diphenylphosphoryl azide (324.7 mg, 1.180 mmol, 1.1 equiv) and triethylamine (169.1 mg, 1.670 mmol, 1.6 eq) in toluene was stirred for 3 h at room temperature under nitrogen atmosphere. To the above mixture was added benzyl alcohol (225.8 mg, 2.088 mmol, 2.0 eq). The resulting mixture was stirred for additional 4 h at 100° C. The resulting mixture was concentrated under vacuum. The residue was purified by reverse flash chromatography with the following conditions: column, silica gel; mobile phase, MeCN in water, 0% to 50% gradient in 20 min; UV 254 nm. This resulted in Compound 6 (280 mg, 68.33%) as a yellow oil. LCMS (ESI) [M+H]⁺: 393.

### Step G: Synthesis of methyl 1-tert-butoxlcarbonyl (3S)-amino-piperidine-(5R)-carboxylate (Compound 7)

A solution of Compound 6 (1 g, 2.548 mmol, 1 eq) and Pd/C (0.1 g, 0.255 mmol, 0.1 eq) in methanol (20 mL) was stirred for 1 h at room temperature under hydrogen atmosphere. The precipitated solids were collected by filtration and washed with MeOH (3×3 mL). The resulting mixture was concentrated under vacuum. This resulted in Compound 7 (600 mg, 91.16%) as a yellow solid. LCMS (ESI) [M+H]⁺: 259.

### Step H: Synthesis of methyl 1-tert-butoxlcarbonyl (3S)-(2-methylpropyl)amino-piperidine (5R)-carboxylate (Compound A)

A solution of Compound 7 (215 mg, 0.751 mmol, 1.0 eq), isobutyraldehyde (64.9 mg, 0.901 mmol, 1.2 eq) and sodium triacetoxyborohydride (477.4 mg, 2.253 mmol, 3.0 eq) in MeOH was stirred for 15 h at room temperature. The residue was purified by reverse flash chromatography with the following conditions: column, silica gel; mobile phase, MeCN in water, 0% to 50% gradient in 20 min; UV 254 nm. This resulted in Compound A (170 mg, 72.00%) as a yellow oil. LCMS (ESI) [M+H]⁺: 315.

### Synthesis of intermediate compound B

### Step A: Synthesis of methyl 5-cyclopropyl-3-fluoropyridine-2-carboxylate (Compound 8)

A solution of methyl 5-bromo-3-fluoropyridine-2-carboxylate (3.3 g, 14.101 mmol, 1.0 eq), cyclopropyltrifluoro-lambda4-borane potassium (6.26 g, 42.303 mmol, 3.0 eq), Pd(dppf)Cl₂ (1.25 g, 1.410 mmol, 0.1 eq) and K₂CO₃ (5.85 g, 42.303 mmol, 3 eq) in 1,4-dioxane and H₂O was stirred for 2 h at 85° C. under nitrogen atmosphere. The resulting mixture was extracted with EtOAc (3×100 mL). The combined organic layers were dried over anhydrous Na₂SO₄. After filtration, the filtrate was concentrated under reduced pressure. This resulted in Compound 8 (1.2 g, 43.60%) as off-white solid. LCMS (ESI) [M+H]⁺: 196.

### Step B: Synthesis of methyl 5-cyclopropyl-3-[3-(trans-methoxy)cyclobutyl]amino-pyridine-2-carboxylate (Compound 9)

A solution of Compound 8 (300 mg, 1.537 mmol, 1.0 eq), (1r,3r)-3-methoxycyclobutan-1-amine (155.5 mg, 1.537 mmol, 1.0 eq) and DIEA (595.9 mg, 4.611 mmol, 3.0 eq) in NMP was stirred for 2 h at 140° C. under nitrogen atmosphere. The residue was purified by reverse flash chromatography with the following conditions: column, silica gel; mobile phase, MeCN in water, 0% to 50% gradient in 20 min, UV 254 nm. This resulted in Compound 9 (196 mg, 46.15%) as a yellow solid. LCMS (ESI) [M+H]⁺: 277.

### Step C: Synthesis of 5-cyclopropyl-3-[3-(trans-methoxy)cyclobutyl]amino-pyridine-2-carboxylate acid (Compound B)

A solution of Compound 9 (196 mg, 0.709 mmol, 1.0 eq) and lithium hydroxide monohydrate (148.8 mg, 3.545 mmol, 5.0 eq) in THF and H₂O was stirred for 2 h at room temperature. The mixture was acidified to pH 5 with AcOH. The residue was purified by reverse flash chromatography with the following conditions: column, silica gel; mobile phase, MeCN in water, 0% to 50% gradient in 20 min, UV 254 nm. This resulted in Compound B (170 mg, 91.37%) as a yellow solid. LCMS (ESI) [M+H]⁺: 263.

### Example 2. (3S)—{N-(2-methylpropyl), N-{5-cyclopropyl-3-[3-(trans-methoxy)cyclobutyl]amino}-pyridine-2-carbonyl}amino-(5R)-(3-fluoroazetidine-N-carbonyl)-piperidine (Compound 10, also termed as 5-cyclopropyl-N-[5-(3-fluoroazetidine-1-carbonyl)piperidin-3-yl]-N-(2-methylpropyl)-3-{[(1r,3r)-3-methoxycyclobutyl]amino}pyridine-2-carboxamide)

### Synthesis of methyl 1-tert-butoxylcarbonyl-(3S)—{N-(2-methylpropyl), N-{5-cyclopropyl-3-[3-(trans-methoxy)cyclobutyl]amino}pyridine-2-carbonyl}amino-piperidine-(5R)-carboxylate

A solution of Compound A (220 mg, 0.700 mmol, 1.0 eq), Compound B (183.5 mg, 0.700 mmol, 1.0 eq), N,N-diisopropylethylamine (271.3 mg, 2.100 mmol, 3.0 eq) and HATU (319.3 mg, 0.840 mmol, 1.2 eq) in DMF was stirred for 2 h at room temperature. The residue was purified by reverse flash chromatography with the following conditions: column, silica gel; mobile phase, MeCN in water, 0% to 50% gradient in 20 min; UV 254 nm. This resulted in methyl 1-tert-butoxylcarbonyl-(3S)—{N-(2-methylpropyl), N-{5-cyclopropyl-3-[3-(trans-methoxy)cyclobutyl]amino}-pyridine-2-carbonyl}amino-piperidine-(5R)-carboxylate (230 mg, 58.83%) as a yellow oil. LCMS (ESI) [M+H]⁺: 559.

### Synthesis of 1-tert-butoxylcarbonyl (3S)-{N-(2-methylpropyl),N-{5-cyclopropyl-3-[3-(trans-methoxy)cyclobutyl]amino}pyridine-2-carbonyl}amino-piperidine-(5R)- carboxylic acid (Compound C)

A solution of methyl 1-tert-butoxylcarbonyl-(3S)—{N-(2-methylpropyl), N-{5-cyclopropyl-3-[3-(trans-methoxy)cyclobutyl]amino}pyridine-2-carbonyl}amino-piperidine-(5R)-carboxylate (578 mg, 1.035 mmol, 1.0 eq) and lithium hydroxide monohydrate (217.1 mg, 5.175 mmol, 5.0 eq) in THF/H₂O was stirred overnight at room temperature. The mixture was acidified to pH 5 with AcOH. The residue was purified by reverse flash chromatography with the following conditions: column, silica gel; mobile phase, MeCN in water, 0% to 50% gradient in 20 min; UV 254 nm. This resulted in Compound C (480 mg, 85.18%) as a yellow solid. LCMS (ESI) [M+H]⁺: 545.

### Synthesis of 1-tert-butoxylcarbonyl (3S)-{N-(2-methylpropyl), N-{5-cyclopropyl-(cyclopropyl-3-[3-(trans-methoxy)cyclobutyl]amino}pyridine-2-carbonyl}amino-(5R)- (3-fluoroazetidinyl-N-carbonyl)-piperidine

A solution of Compound C (100 mg, 0.184 mmol, 1.0 eq), 3-fluoroazetidine (15.2 mg, 0.202 mmol, 1.1 eq), N,N-diisopropylethylamine (71.2 mg, 0.552 mmol, 3.0 eq) and HATU (53.1 mg, 0.221 mmol, 1.2 eq) in DMF was stirred for 2 h at room temperature. The residue was purified by reverse flash chromatography with the following conditions: column, silica gel; mobile phase, MeCN in water, 0% to 50% gradient in 20 min; UV 254 nm. This resulted in 1-tert-butoxylcarbonyl-(3S)—{N-(2-methylpropyl), N-{5-cyclopropyl-3-[3-(trans-methoxy)cyclobutyl]amino}-pyridine-2-carbonyl}amino-(5R)-(3-fluoroazetidinyl-N-carbonyl)-piperidine (95 mg, 85.99%) as a yellow oil. LCMS (ESI) [M+H]⁺: 602

### Synthesis of Compound 10

A solution of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(3-fluoroazetidinyl-N-carbonyl)-piperidine (100 mg, 0.166 mmol, 1.0 equiv) in DCM and TFA was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 10 (18.4 mg, 18.40%) as a white solid. LCMS (ESI) [M+H]⁺: 502.30. ¹H NMR (400 MHz, DMSO-d6) δ 7.63 (d, J=19.6 Hz, 1H), 6.46 (d, J=7.8 Hz, 1H), 5.74-5.23 (m, 2H), 4.62-3.58 (m, 7H), 3.20-3.24 (m, 2H), 3.14 (s, 3H), 3.01-2.64 (m, 2H), 2.42-1.46 (m, 11H), 0.94 (m, 6H), 0.76 (d, J=5.5 Hz, 2H), 0.60 (s, 2H).

### Example 3, (3S)-{N-(2-methylpropyl), N-{5cyclopropyl-3-[3-(trans-methoxy)cyclobutyl]amino}-pyridine-2-carbonyl}amino-(5R)-(3,3-difluoropyrrolidine-N- carbonyl)-piperidine (Compound 11, also termed as 5-cyclopropyl-N-[5-(3,3-difluoropyrrolidine-1-carbonyl)piperidin-3-yl]-N-(2-methylpropyl)-3-{[(1r,3r)-3- methoxycyclobutyl]amino}pyridine-2-carboxamide)

### Synthesis of 1-tert-butoxylcarbonyl (3S)-{N-(2-methylpropyl), N-{5-cyclopropyl-3-[3-(trans-methoxy)cyclobutyl]amino}pyridine-2-carbonyl}amino-(5R)-(3- carbonyl)-piperidine

A solution of Compound C (100 mg, 0.184 mmol, 1.0 eq), 3,3-difluoropyrrolidine (23.6 mg, 0.221 mmol, 1.2 eq), N,N-diisopropylethylamine (71.2 mg, 0.552 mmol, 3.0 eq) and HATU (83.8 mg, 0.221 mmol, 1.2 eq) in DMF was stirred for 2 h at room temperature. The residue was purified by reverse flash chromatography with the following conditions: column, silica gel; mobile phase, MeCN in water, 0% to 50% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3,3-difluoropyrrolidine-N-carbonyl)-piperidine (98 mg, 84.22%) as a yellow oil. LCMS (ESI) [M+H]⁺: 634.

### Synthesis of Compound 11

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3,3-difluoropyrrolidine-N-carbonyl)-piperidine (96 mg, 0.151 mmol, 1.0 eq) in DCM/TFA was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min; UV 254 nm. This resulted in Compound 11 (50 mg, 61.85%) as a white solid. LCMS (ESI) [M+H]⁺: 534.30. ¹H NMR (400 MHz, DMSO-d6) δ 7.63 (d, J=12.7 Hz, 1H), 6.58-6.37 (m, 1H), 5.69-5.46 (m, 1H), 4.17-3.79 (m, 4H), 3.66 (d, J=13.2 Hz, 2H), 3.53-3.42 (m, 1H), 3.29-3.18 (m, 2H), 3.14 (s, 3H), 3.03-2.67 (m, 3H), 2.56 (d, J=17.4 Hz, 2H), 2.40-2.21 (m, 5H), 2.03 (s, 3H), 1.94-1.84 (m, 2H), 1.82-1.49 (m, 1H), 1.07-0.84 (m, 6H), 0.75 (d, J=5.4 Hz, 2H), 0.65-0.50 (m, 2H).

### Example 4(3S)-{N-(2-methylpropyl), N-{5cyclopropyl-3-[3-(trans-methoxy)cyclobutyl]amino}-pyridine-2-carbonyl}amino-(5R)-(4,4-difluoropyrrolidine-N- carbonyl)-piperidine (Compound 12, also termed as 5-cyclopropyl-N-[5-(4,4-difluoropyrrolidine-1-carbonyl)piperidin-3-yl]-N-(2-methylpropyl)-3-{[(1r,3r)-3- methoxycyclobutyl]amino}pyridine-2-carboxamide)

### Synthesis of 1-tert-butoxylcarbonyl-(3S)-{N-(2-methylpropyl), N-{5-cyclopropyl-3-[3-(trans-methoxy)cyclobutyl]amino}pyridine-2-carbonyl}amino-(5R)-(4,4-difluoropiperidine-N-carbonyl)-piperidine

A solution of Compound C (100 mg, 0.184 mmol, 1.0 eq), 4,4-difluoropiperidine (26.7 mg, 0.221 mmol, 1.2 eq), N,N-diisopropylethylamine (71.2 mg, 0.552 mmol, 3.0 eq) and HATU (53.1 mg, 0.221 mmol, 1.2 eq) in DMF was stirred for 1h at room temperature. The residue was purified by reverse flash chromatography with the following conditions: column, silica gel; mobile phase, MeCN in water, 0% to 50% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}-pyridine-2-carbonyl}amino-(5R)-(4,4-difluoropiperidine-N-carbonyl)-piperidine (96 mg, 80.72%) as a yellow oil. LCMS (ESI) [M+H]⁺: 648.

### Synthesis of Compound 12

A solution of 1-tert-butoxycarbonyl-(3S)—{N-(2-methylpropyl), N-{5-cyclopropyl-3-[3-(trans-methoxy)cyclobutyl]amino}-pyridine-2-carbonyl}amino-(5R)-(4,4-difluoropiperidine-N-carbonyl)-piperidine (98 mg, 0.151 mmol, 1.0 eq) in DCM/TFA was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min; UV 254 nm. This resulted in Compound 12 (50 mg, 60.35%) as a white solid. LCMS (ESI) [M+H]⁺: 548.35. ¹H NMR (400 MHz, DMSO-d6) δ 7.63 (d, J=13.3 Hz, 1H), 6.46 (d, J=8.7 Hz, 1H), 5.55 (m, 1H), 3.94 (m, 3H), 3.77-3.46 (m, 5H), 3.23 (m, 2H), 3.14 (s, 3H), 3.01-2.53 (m, 4H), 2.29 (m, 4H), 2.16-1.69 (m, 10H), 1.05-0.85 (m, 6H), 0.76 (d, J=5.7 Hz, 2H), 0.61 (d, J=6.4 Hz, 2H).

### Example 5(3S)-{N-(2-methylpropyl), N-{5cyclopropyl-3-[3-(trans-methoxy)cyclobutyl]amino}-pyridine-2-carbonyl}amino-(5R)-(4,4-trifluoromethoxypiperidine- N- carbonyl)-peperridine (Compound 13, also termed as 5-cyclopropyl-N-(2-methylpropyl)-3-{[(1r,3r)-3-methoxycyclobutyl]amino}-N-{5-[4-(trifluoromethoxy)piperdine- 1-carbonyl]piperidin-3-yl}pyridine-2-carboxamide)

### Synthesis of N-tert-butoxycarbonyl 4-(trifluoromethoxy) piperidine

A solution of N-tert-butoxycarbonyl 4-hydroxypiperidine (2 g, 9.937 mmol, 1 eq), oxo(trifluoromethanesulfonyl)silver (10.21 g, 39.748 mmol, 4 eq), potassium fluoride (2.31 g, 39.748 mmol, 4 eq), 2-fluoropyridine (2.89 g, 29.811 mmol, 3 eq), trifluoromethyltrimethylsilane (4.24 g, 29.811 mmol, 3 eq) and selectfluor (15.84 g, 44.716 mmol, 4.5 eq) in EtOAc was stirred for 15 h at room temperature under nitrogen atmosphere. The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with petroleum ether/ethyl ether (4:1) to afford N-tert-butoxycarbonyl 4-(trifluoromethoxy) piperidine (1.6 g, 59.80%) as a yellow oil. LCMS (ESI) [M+H]⁺: 270. ¹H NMR (300 MHz, CDCl₃) δ 4.43 (m, 1H), 3.71 (m, 2H), 3.29 (m, 2H), 1.91 (m, 2H), 1.75 (m, 2H), 1.48 (s, 9H). ¹⁹F NMR (282 MHz, CDCl₃) δ−57.94 (s, 3F).

### Synthesis of 4-(trifluoromethoxy)piperidine

A solution of N-tert-butoxycarbonyl 4-(trifluoromethoxy) piperidine (100 mg, 0.371 mmol, 1 eq) in a solution of HCl in 1,4-dioxane was stirred for 1 h at room temperature. The resulting mixture was concentrated under vacuum. This resulted in 4-(trifluoromethoxy)piperidine (70 mg, crude) as a yellow solid. LCMS (ESI) [M+H]⁺: 170.

### Synthesis of 1-tert-butoxylcarbonyl-(3S)-{N-(2-methylpropyl), N-{5-cyclopropyl-3-[3-(trans-methoxy)cyclobutyl]amino}pyridine-2-carbonyl}amino-(5R)-(4,-trifluoromethoxypiperidine-N-carbonyl)-piperidine

A solution of Compound C (100 mg, 0.184 mmol, 1 eq), 4-(trifluoromethoxy)piperidine (70 mg, crude), N,N-diisopropylethylamine (118.6 mg, 0.920 mmol, 5 eq) and HATU (83.8 mg, 0.221 mmol, 1.2 eq) in DMF was stirred for 2 h at room temperature. The residue was purified by reverse flash chromatography with the following conditions: column, silica gel; mobile phase, MeCN in water, 0% to 50% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(4-trifluoromethoxypiperidine-N-carbonyl)-piperidine (110 mg, 86.11%) as a yellow oil. LCMS (ESI) [M+H]⁺: 696.

### Synthesis of Compound 13

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(4-trifluoromethoxypiperidine-N-carbonyl)-piperidine (100 mg, 0.144 mmol, 1 eq) and a solution of HCl in 1,4-dioxane (4 mL) was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 13 (30 mg, 35.04%) as a white solid. LCMS (ESI) [M+H]⁺: 596.3. ¹H NMR (300 MHz, DMSO-d6) δ 7.63 (s, 1H), 6.48 (s, 1H), 5.55 (d, J=34.8 Hz, 1H), 4.67 (s, 1H), 4.18-3.58 (m, 5H), 3.14 (m, 7H), 2.99-2.68 (m, 3H), 2.57 (m, 1H), 2.31 (m, 4H), 2.15-1.85 (m, 8H), 1.56 (m, 2H), 0.95 (m, 6H), 0.76 (d, J=4.9 Hz, 2H), 0.61 (d, J=6.4 Hz, 2H).

### Example 6. (3S)-{N-(2-methylpropyl), N-{5cyclopropyl-3-[3-(trans-methoxy)cyclobutyl]amino}-pyridine-2-carbonyl}amino-(5R)-[3R)-methylmorpholine-N- carbonyl]-piperidine (Compound 14, also termed as 5-cyclopropyl-N-{5-[3R)-3-methylmorpholine-4-carbonyl]piperidin-3-yl}-N-(2methylpropyl)-3-{[(1r,3r)-3-methoxycyclobutyl]amino}pyridine-2-carboxamide)

### Synthesis of 1-tert-butoxylcarbonyl-(3S)-{N-(2-methylpropyl), N-{5-cyclopropyl-3-[3-(trans-methoxy)cyclobutyl]amino}pyridine-2-carbonyl}amino-(5R)-[3R)-methylmorpholine-N-carbonyl]-piperidine

To a stirred mixture of Compound C (120 mg, 0.220 mmol, 1.0 eq), (3R)-3-methylmorpholine (26.7 mg, 0.264 mmol, 1.2 eq), and DIEA (85.4 mg, 0.660 mmol, 3.0 eq) in DMF (2 mL) was added HATU (100.5 mg, 0.264 mmol, 1.2 eq) at room temperature under nitrogen atmosphere. The resulting mixture was stirred for additional 1 h at room temperature. The residue was purified by reverse flash chromatography with the following conditions: column, silica gel; mobile phase, MeCN in water, 10% to 60% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-[(3R)-methylmorpholine-N-carbonyl]-piperidine (110 mg, 79.53%) as a yellow oil. LCMS (ESI) [M+H]⁺: 629.

### Synthesis of Compound 14

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-[(3R)-methylmorpholine-N-carbonyl]-piperidine (100 mg, 0.159 mmol, 1 eq) in dioxane (1 mL) was added HCl-dioxane (1 mL) at room temperature under nitrogen atmosphere. The resulting mixture was stirred for additional 2 h at room temperature. The resulting mixture was concentrated under vacuum. The mixture was neutralized to pH 8 with saturated Na₂CO₃ (aq.). The residue was purified by reverse flash chromatography with the following conditions: column, silica gel; mobile phase, MeCN in water, 10% to 70% gradient in 20 min; detector, UV 254 nm. This resulted in Compound 14 (26.0 mg, 30.93%) as a white solid. LCMS (ESI) [M+H]⁺: 528.40. ¹H NMR (300 MHz, DMSO-d6) δ 7.62 (s, 1H), 6.49 (s, 1H), 5.54 (d, J=34.1 Hz, 1H), 4.33 (s, 1H), 4.17-3.90 (m, 3H), 3.90-3.75 (m, 1H), 3.76-3.55 (m, 2H), 3.53-3.37 (m, 3H), 3.15 (s, 3H), 3.02-2.64 (m, 4H), 2.56 (s, 1H), 2.38-2.20 (m, 3H), 2.17-1.65 (m, 6H), 1.36-0.49 (m, 14H).

### Example 7. (3S)-{N-(2-methylpropyl), N-{5cyclopropyl-3-[3-(trans-methoxy)cyclobutyl]amino}-pyridine-2-carbonyl}amino-(5R)-[3R)-methylmorpholine-N- carbonyl]-piperidine (Compound 15, also termed as 5-cyclopropyl-N-{5-[3R)-3-methylmorpholine-4-carbonyl]piperidin-3-yl}-N-(2methylpropyl)-3-{[(1r,3r)-3-methoxycyclobutyl]amino}pyridine-2-carboxamide)

### Synthesis of 1-tert-butoxylcarbonyl-(3S)-{N-(2-methylpropyl), N-{5-cyclopropyl-3-[3-(trans-methoxy)cyclobutyl]amino}pyridine-2-carbonyl}amino-(5R)-[3R)-methylmorpholine-N-carbonyl]-piperidine

To a stirred mixture of 1-tert-butoxylcarbonyl-(3S)—{N-(2-methylpropyl), N-{5-cyclopropyl-3-[(1R,3R)-3-methoxycyclobutyl]amino-pyridine-2-carbonyl}}amino-piperidine-(5R)-carboxylic acid (120 mg, 0.220 mmol, 1 eq), (3S)-3-methylmorpholine (26.7 mg, 0.264 mmol, 1.2 eq), and DIEA (85.4 mg, 0.660 mmol, 3.0 eq) in DMF (2 mL) was added HATU (100.5 mg, 0.264 mmol, 1.2 eq) at room temperature under nitrogen atmosphere. The resulting mixture was stirred for additional 1 h at room temperature. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-[(3S)-methylmorpholine-N-carbonyl]-piperidine (100 mg, 72.30%) as a yellow oil. LCMS (ESI) [M+H]⁺: 629.

### Synthesis of Compound 15

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-[(3S)-methylmorpholine-N-carbonyl]-piperidine (100 mg, 0.159 mmol, 1.0 eq) in dioxane (1 mL) was added HCl-dioxane (1 mL) at room temperature under nitrogen atmosphere. The resulting mixture was stirred for additional 2 h at room temperature. The resulting mixture was concentrated under vacuum. The mixture was neutralized to pH 8 with saturated Na₂CO₃ (aq.). The residue was purified by reverse flash chromatography with the following conditions: column, silica gel; mobile phase, MeCN in water, 0% to 60% gradient in 20 min, UV 254 nm. This resulted in Compound 15 (48.5 mg, 57.70%) as a white solid. LCMS (ESI) [M+H]⁺: 528.40. ¹H NMR (300 MHz, DMSO-d6) δ 7.62 (s, 1H), 6.48 (s, 1H), 5.55 (d, J=32.6 Hz, 1H), 4.15-3.88 (m, 3H), 3.82 (d, J=8.7 Hz, 1H), 3.61 (d, J=12.0 Hz, 2H), 3.15 (s, 6H), 3.03-2.63 (m, 3H), 2.61-2.54 (m, 2H), 2.40-2.16 (m, 4H), 2.03 (s, 3H), 1.96-1.73 (m, 3H), 1.35-0.51 (m, 14H).

### Example 8. (3S)-{N-(2-methylpropyl), N-{5cyclopropyl-3-[3-(trans-methoxy)cyclobutyl]amino}-pyridine-2-carbonyl}amino-(5R)-2,2-dimethylmorpholine-N- carbonyl)-piperidine (Compound 16, also termed as 5-cyclopropyl-N-[5-(2,2-dimethylmorpholine-4-carbonyl)piperidin-3-yl]-N-(2-methylpropyl)-3-{[(1r,3r)-3- methoxycyclobutyl]amino}pyridine-2-carboxamide)

### Synthesis of 1-tert-butoxylcarbonyl-(3S)-{N-(2-methylpropyl), N-{5-cyclopropyl-3-[3-(trans-methoxy)cyclobutyl]amino}pyridine-2-carbonyl}amino-(5R)-(2,2dimethylmorpholine-N-carbonyl)-piperidine

To a stirred mixture of Compound C (120 mg, 0.220 mmol, 1.0 eq), (3R)-3-methylmorpholine (26.7 mg, 0.264 mmol, 1.2 eq) and DIEA (85.4 mg, 0.660 mmol, 3.0 eq) in DMF (2 mL) was added HATU (100.5 mg, 0.264 mmol, 1.2 eq) at room temperature under nitrogen atmosphere. The resulting mixture was stirred for additional 1 h at room temperature. The residue was purified by reverse flash chromatography with the following conditions: column, silica gel; mobile phase, MeCN in water, 0% to 70% gradient in 20 min, UV 254 nm. This resulted in 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(2,2-dimethylmorpholine-N-carbonyl)-piperidine (110 mg, 79.53%) as a yellow oil. LCMS (ESI) [M+H]⁺: 643.

### Synthesis of (3S,5R)-3-{N-(2-methylpropyl), N-{5-cyclopropyl-3-[3-(trans-methoxy)cyclobutyl]amino}pyridine2-carbonyl}amino-5-(2,2-dimethylmorpholine- N-carbonyl)-piperidine Compound 16

To a stirred mixture of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(2,2-dimethylmorpholine-N-carbonyl)-piperidine (100 mg, 0.156 mmol, 1.0 eq) in dioxane (1 mL) was added a solution of HCl in dioxane (1 mL) at room temperature under nitrogen atmosphere. The resulting mixture was stirred for additional 2 h at room temperature. The resulting mixture was concentrated under vacuum. The mixture was neutralized to pH 8 with saturated Na₂CO₃ (aq.). The residue was purified by reverse flash chromatography with the following conditions: column, silica gel; mobile phase, MeCN in water, 0% to 70% gradient in 20 min, UV 254 nm. This resulted in Compound 16 (54.8 mg, 64.93%) as a white solid. LCMS (ESI) [M+H]⁺: 542.4. ¹H NMR (300 MHz, DMSO-d6) δ 7.62 (s, 1H), 6.48 (s, 1H), 5.55 (d, J=39.0 Hz, 1H), 4.04-3.85 (m, 2H), 3.73-3.37 (m, 4H), 3.04-2.55 (s, 3H), 3.04-2.55 (m, 4H), 2.38-2.19 (m, 4H), 2.17-1.68 (m, 7H), 1.21-1.04 (m, 6H), 1.00 (d, J=7.9 Hz, 2H), 0.90 (d, J=6.6 Hz, 4H) 0.75 (s, 2H), 0.61 (d, 2H).

### Example 9. (3S)-{N-(2-methylpropyl), N-{5cyclopropyl-3-[3-(trans-methoxy)cyclobutyl]amino}-pyridine-5-carbonyl}amino-(5R)-(morpholine-N-carbonyl)-piperidine (Compound 17, also termed as 2-tert-butyl-N-2-((2-methylpropyl)-N-[5-(morpholine-4-carbonyl)pyrimidin-3-yl]-4-{[(1r,3r)-3-methoxycyclobutyl]amino}pyrimidine-5-carboxamide)

### Synthesis of ethyl 2-tert-butyl-4-oxo-5H-pyrimidine-5-carboxylate

A solution of 2,2-dimethylpropanimidamide (200 mg, 1.997 mmol, 1.0 eq), 1,3-diethyl 2-(ethoxymethylidene) propanedioate (518.1 mg, 2.396 mmol, 1.2 eq) and EtONa (135.8 mg, 1.997 mmol, 1.0 eq) in EtOH (3 mL) was stirred for 1 h at room temperature under nitrogen atmosphere. The residue was acidified to pH 6 with conc. HCl. The residue was washed with ethyl acetate (3×10 mL). The resulting mixture was concentrated under vacuum. The residue was purified by silica gel column chromatography, eluted with petroleum ether/EtOAc (1:1) to afford ethyl 2-tert-butyl-4-oxo-5H-pyrimidine-5-carboxylate (185 mg, 41.31%) as a yellow solid. LCMS (ESI) [M+H]⁺: 225.

### Synthesis of ethyl 2-tert-butyl-4-chloropyridimine-5-carboxylate

A mixture of ethyl 2-tert-butyl-4-oxo-5H-pyrimidine-5-carboxylate (100 mg, 0.446 mmol, 1.0 eq) and phosphorus oxychloride (341.8 mg, 2.230 mmol, 5.0 eq) in triethylamine (2 mL) was stirred for 30 min at 100° C. under nitrogen atmosphere. The residue was washed with water (3×3 mL). The resulting mixture was concentrated under vacuum. The residue was purified by reverse flash chromatography with the following conditions: column, silica gel; mobile phase, MeCN in water, 0% to 60% gradient in 20 min, UV 254 nm. This resulted in ethyl 2-tert-butyl-4-chloropyrimidine-5-carboxylate (70 mg, 64.68%) as colorless oil. LCMS (ESI) [M+H]⁺: 244.

### Synthesis of ethyl 2-tert-butyl-4-[3-(trans-methoxy)cyclobutyl]amino-pyrimidine-5-carboxylate

To a stirred mixture of ethyl 2-tert-butyl-4-chloropyrimidine-5-carboxylate (400 mg, 1.648 mmol, 1.0 eq) and 3-trans-methoxycyclobutan-1-amine (183.4 mg, 1.813 mmol, 1.1 eq) in NMP (4 mL) were added DIEA (639.1 mg, 4.944 mmol, 3.0 eq). The mixture was stirred at 140° C. under nitrogen atmosphere for 2 h. The residue was purified by reverse flash chromatography with the following conditions: column, silica gel; mobile phase, MeCN in water, 0% to 60% gradient in 20 min, UV 254 nm. This resulted in ethyl 2-tert-butyl-4-[(3-trans-methoxy)cyclobutyl]amino-pyrimidine-5-carboxylate (280 mg, 55.27%) as a red oil. LCMS (ESI) [M+H]⁺: 308.

### Synthesis of ethyl 2-tert-butyl-4-[3-(trans-methoxy)cyclobutyl]amino-pyrimidine-5-carboxylate acid

To a stirred mixture of ethyl 2-tert-butyl-4-[(3-trans-methoxy)cyclobutyl]amino-pyrimidine-5-carboxylate (270 mg, 0.878 mmol, 1.0 eq) and LiOH (184.3 mg, 4.390 mmol, 5.0 eq) in THF (2 mL) were added H₂O (2.0 mL) at 25° C. under nitrogen atmosphere. The mixture was stirred at room temperature for 1 h. The mixture was acidified to pH 6 with citric acid. The residue was purified by reverse flash chromatography with the following conditions: column, silica gel; mobile phase, MeCN in water, 0% to 70% gradient in 20 min, UV 254 nm. This resulted in 2-tert-butyl-4-[(3-trans-methoxy)cyclobutyl]amino-pyrimidine-5-carboxylic acid (220 mg, 89.66%) as a yellow solid. LCMS (ESI) [M+H]⁺: 280

### Synthesis of methyl 1-tert-butoxylcarbonyl-(3S)-{N-(2-methylpropyl), N-{2-tert-butyl-4[3-trans-methoxy)cyclobutyl]amino}pyrimidine-5-carbonyl}amino-piperidine-(5R)-carboxylate

To a stirred solution of Compound A (200 mg, 0.636 mmol, 1.0 eq), 2-tert-butyl-4-[(3-trans-methoxy)cyclobutyl]amino-pyrimidine-5-carboxylic acid (213.2 mg, 0.763 mmol, 1.2 eq) and DIEA (246.6 mg, 1.908 mmol, 3.0 eq) in DMF (3 mL) was added HATU (290.2 mg, 0.763 mmol, 1.2 eq) at room temperature under nitrogen atmosphere. The resulting mixture was stirred for additional 2 h at room temperature. The residue was purified by reverse flash chromatography with the following conditions: column, silica gel; mobile phase, MeCN in water, 0% to 60% gradient in 20 min, UV 254 nm. This resulted in methyl 1-tert-butoxylcarbonyl-(3S)—{N-(2-methylpropyl), N-{2-tert-butyl-4-[(3-trans-methoxy)cyclobutyl]amino}pyrimidine-5-carbonyl}amino-piperidine-(5R)-carboxylate (240 mg, 65.53%) as a yellow oil. LCMS (ESI) [M+H]⁺: 577.

### Synthesis of 1-tert-butoxylcarbonyl-(3S)-{N-(2-methylpropyl), N-{2-tert-butyl-4-[(3-trans-methoxy)cyclobutyl]amino}pyrimidine-5-carbonyl}amino-piperidine-(5R)- carboxylic acid

To a stirred solution of methyl 1-tert-butoxylcarbonyl-(3S)—{N-(2-methylpropyl), N-{2-tert-butyl-4-[(3-trans-methoxy)cyclobutyl]amino}pyrimidine-5-carbonyl}amino-piperidine-(5R)-carboxylate (160 mg, 0.278 mmol, 1.0 eq) and lithium hydrate (58.3 mg, 1.390 mmol, 5.0 eq) in THF (2 mL) was added H₂O (2 mL) at room temperature. The resulting mixture was stirred for additional 1 h at room temperature. The mixture was acidified to pH 6 with citric acid. The residue was purified by reverse flash chromatography with the following conditions: column, silica gel; mobile phase, MeCN in water, 0% to 50% gradient in 20 min, UV 254 nm. This resulted in 1-tert-butoxylcarbonyl-(3S)—{N-(2-methylpropyl), N-{2-tert-butyl-4-[(3-trans-methoxy)cyclobutyl]amino}pyrimidine-5-carbonyl}amino-piperidine-(5R)-carboxylic acid (150 mg, 96.09%) as a yellow oil. LCMS (ESI) [M+H]⁺: 563.

### Synthesis of 1-tert-butoxycarbonyl-(3S)-{N-(2-methylproprl), N-{2-tert-butyl-4-[(3-trans-methoxy)cyclobutyl]amino}pyrimidine-5-carbonyl}amino-(5R)-(morpholine-N- carbonyl)-piperidine

To a stirred mixture of 1-tert-butoxylcarbonyl-(3S)—{N-(2-methylpropyl), N-{2-tert-butyl-4-[(3-trans-methoxy)cyclobutyl]amino}pyrimidine-5-carbonyl}amino-piperidine-(5R)-carboxylic acid (130 mg, 0.231 mmol, 1.0 eq), morpholine (24.2 mg, 0.277 mmol, 1.2 eq) and DIEA (89.7 mg, 0.693 mmol, 3.0 eq) in DMF (3 mL) was added HATU (105.6 mg, 0.277 mmol, 1.2 eq) at room temperature under nitrogen atmosphere. The resulting mixture was stirred for additional 1 h at room temperature. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-(2-methylpropyl), N-{2-tert-butyl-4-[(3-trans-methoxy)cyclobutyl]amino}pyrimidine-5-carbonyl}amino-(5R)-(morpholine-N-carbonyl)-piperidine (140 mg, 95.89%) as a yellow oil. LCMS (ESI) [M+H]⁺: 632.

### Synthesis of Compound 17

To a stirred mixture of 1-tert-butoxycarbonyl-(3S)—{N-(2-methylpropyl), N-{2-tert-butyl-4-[(3-trans-methoxy)cyclobutyl]amino}pyrimidine-5-carbonyl}amino-(5R)-(morpholine-N-carbonyl)-piperidine (80 mg, 0.127 mmol, 1.0 eq) in DCM (1 mL) was added TFA (1 mL) at 25° C. The resulting mixture was stirred for additional 1 h at 25° C. The resulting mixture was concentrated under vacuum. The mixture was neutralized to pH 8 with saturated Na₂CO₃ (aq.). The residue was purified by reverse flash chromatography with the following conditions: column, silica gel; mobile phase, MeCN in water, 0% to 60% gradient in 20 min, UV 254 nm. This resulted in Compound 17 (36.3 mg, 53.93%) as a white solid. LCMS (ESI) [M+H]⁺: 531.70. ¹H NMR (300 MHz, DMSO-d6) δ 8.02 (s, 1H), 6.95 (s, 1H), 4.65-4.43 (m, 1H), 3.93 (d, J=5.3, 4.8 Hz, 1H), 3.70 (s, 1H), 3.48 (d, J=29.8 Hz, 8H), 3.15 (s, 5H), 2.93 (d, J=7.5 Hz, 1H), 2.84-2.55 (m, 3H), 2.39-2.15 (m, 6H), 2.03-1.75 (m, 3H), 1.29 (s, 9H), 0.82 (d, J=6.5 Hz, 6H).

### Example 10. (3S)-{N-(2-methylpropyl), N-{5cyclopropyl-3-[3-(trans-methoxy)cyclobutyl]amino}-pyridine-2-carbonyl}amino-(5R)-(morpholine-N-carbonyl)-piperidine (Compound 18, also termed as 5-cyclopropyl-N-(2-methylpropyl)-N[5-(morpholine-4-carbonyl)piperidin-3-yl]3-{[(1r,3r)-3-methoxyxcyclobutyl]amino}pyridine-2-carboxamide)

### Synthesis of 1-tert-butoxycarbonyl-(3S)-{N-(2-methylproprl), N-{5-cyclopropyl-3-[(3-trans-methoxy)cyclobutyl]amino}pyrimidine-2-carbonyl}amino-(5R)-(morpholine- N-carbonyl)-piperidine

A solution of Compound C (100 mg, 0.184 mmol, 1.0 eq), morpholine (19.2 mg, 0.221 mmol, 1.2 eq), N,N-diisopropylethylamine (71.2 mg, 0.552 mmol, 3.0 eq) and HATU (83.8 mg, 0.221 mmol, 1.2 eq) in DMF was stirred for 2 h at room temperature. The residue was purified by reverse flash chromatography with the following conditions: column, silica gel; mobile phase, MeCN in water, 0% to 50% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(morpholine-N-carbonyl)-piperidine (98 mg, 86.97%) as a yellow oil. LCMS (ESI) [M+H]⁺: 614.

### Synthesis of Compound 18

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(morpholine-N-carbonyl)-piperidine (100 mg, 0.163 mmol, 1.0 eq) in DCM and TFA was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 18 (37.2 mg, 44.45%) as a white solid. LCMS (ESI) [M+H]⁺: 514.3. ¹H NMR (400 MHz, DMSO-d6) δ 7.62 (d, J=6.1 Hz, 1H), 6.46 (d, J=7.4 Hz, 1H), 5.60 (d, J=5.9 Hz, 1H), 3.95 (m, 2H), 3.53 (m, 6H), 3.32 (s, 3H), 3.21 (m, 2H), 3.14 (s, 3H), 3.02-2.66 (m, 4H), 2.54 (m, 1H), 2.41-1.68 (m, 9H), 0.94 (m, 6H), 0.75 (m, 2H), 0.60 (d, J=6.4 Hz, 2H).

### Example 11. (3S)-{N-(2-methylpropyl), N-{5-cyclopropyl-3-[3-(cis-methoxy) cyclobutyl]amino}-pyridine-2-carbonyl}amino-(5R)-(morpholine-N-carbonyl)-piperidine (Compound 19, also termed as 5-cyclopropyl-N-(2-methylpropyl)-N-[5-(morpholine -4-carbonyl)piperidin-3-yl]-3-{[(1s,3s)-3-methoxycyclobutyl]amino}pyridine-2-carboxamide)

### Synthesis of methyl 5-cyclopropyl-3-[3-(cis-methoxy)cyclobutyl]amino-pyridine-2-carboxylate

To a stirred solution of methyl 5-cyclopropyl-3-fluoropyridine-2-carboxylate (100 mg, 0.512 mmol, 1 eq) and 3-cis-methoxycyclobutan-1-amine (62.1 mg, 0.614 mmol, 1.2 eq) in NMP were added DIEA (198.6 mg, 1.536 mmol, 3 eq) at room temperature. The resulting mixture was stirred for 1 h at 140° C. under nitrogen atmosphere. The residue was purified by reverse flash chromatography with the following conditions: column, silica gel; mobile phase, MeCN in water, 0% to 60% gradient in 20 min, UV 254 nm. This resulted in methyl 5-cyclopropyl-3-[3-(cis-methoxy)cyclobutyl]amino-pyridine-2-carboxylate (110 mg, 77.70%) as a yellow oil. LCMS (ESI) [M+H]⁺: 277.

### Synthesis of methyl 5-cyclopropyl-3-[3-(cis-methoxy)cyclobutyl]amino-pyridine-2-carboxylic acid

To a stirred solution of methyl 5-cyclopropyl-3-[3-(cis-methoxy)cyclobutyl]amino-pyridine-2-carboxylate (100 mg, 0.362 mmol, 1 eq) in THF/H₂O was added LiOH (43.3 mg, 1.810 mmol, 5 eq) at room temperature. The resulting mixture was stirred for 1 h at room temperature. The residue was acidified to pH 6 with citric acid. The residue was purified by reverse flash chromatography with the following conditions: column, silica gel; mobile phase, MeCN in water, 0% to 60% gradient in 20 min, UV 254 nm. This resulted in 5-cyclopropyl-3-[3-(cis-methoxy)cyclobutyl]amino-pyridine-2-carboxylic acid (89 mg, 93.76%) as a yellow oil. LCMS (ESI) [M+H]⁺: 263.

### Synthesis of 1-tert-butoxycarbonyl-(3S)-{N-(2-methylpropyl), N-{5-cyclopropyl-3-[3-(cis- methoxy)cyclobutyl]amino}pyridine-2-carbonyl}amino-piperidine-(5R)-carboxylate

To a stirred solution of 5-cyclopropyl-3-[3-(cis-methoxy)cyclobutyl]amino}pyridine-2-carboxylic acid (100 mg, 0.381 mmol, 1 eq), Compound A (143.8 mg, 0.457 mmol, 1.2 eq) and DIEA (147.8 mg, 1.143 mmol, 3 eq) in DMF were added HATU (173.9 mg, 0.457 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 60% gradient in 20 min, UV 254 nm. This resulted in methyl 1-tert-butoxycarbonyl-(3S)—{N-(2-methylpropyl), N-{5-cyclopropyl-3-[3-(cis-methoxy)cyclobutyl]amino}pyridine-2-carbonyl}amino-piperidine-(5R)-carboxylate (110 mg, 51.64%) as a yellow oil. LCMS (ESI) [M+H]⁺: 559.

### Synthesis of 1-tert-butoxycarbonyl-(3S)-{N-(2-methylpropyl), N-{5-cyclopropyl-3-[3-(cis-methoxy) cyclobutyl]amino}pyridine-2-carbonyl}amino-piperidine-(5R)-carboxylic acid

To a stirred solution of methyl 1-tert-butoxycarbonyl-(3S)—{N-(2-methylpropyl), N-{5-cyclopropyl-3-[3-(cis-methoxy)cyclobutyl]amino}pyridine-2-carbonyl}amino-piperidine-(5R)-carboxylate (100 mg, 0.179 mmol, 1 eq) in THF/H₂O were added LiOH (21.4 mg, 0.895 mmol, 5 eq) at room temperature. The resulting mixture was stirred for 1 h at room temperature under nitrogen atmosphere. The residue was acidified to pH 6 with citric acid. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-(2-methylpropyl), N-{5-cyclopropyl-3-[3-(cis-methoxy)cyclobutyl]amino}pyridine-2-carbonyl}amino-piperidine-(5R)-carboxylic acid (89 mg, 91.29%) as a yellow oil. LCMS (ESI) [M+H]⁺: 545.

### Synthesis of 1-tert-butoxycarbonyl-(3S)-{N-(2-methylpropyl), N-{5-cyclopropyl-3-[3-(cis-methoxy) cyclobutyl]amino}pyridine-2-carbonyl}amino-(5R)-(morpholine-N-carbonyl)-piperidine

To a stirred solution of 1-tert-butoxycarbonyl-(3S)—{N-(2-methylpropyl), N-{5-cyclopropyl-3-[3-(cis-methoxy)cyclobutyl]amino}pyridine-2-carbonyl}amino-piperidine-(5R)-carboxylic acid (100 mg, 0.184 mmol, 1 eq), morpholine (19.1 mg, 0.221 mmol, 1.2 eq) and DIEA (28.4 mg, 0.221 mmol, 1.2 eq) in DMF were added HATU (209.42 mg, 0.552 mmol, 3 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-(2-methylpropyl), N-{5-cyclopropyl-3-[3-(cis-methoxy)cyclobutyl]amino}pyridine-2-carbonyl}amino-(5R)-(morpholine-N-carbonyl)-piperidine (102 mg, 90.52%) as a yellow oil. LCMS (ESI) [M+H]⁺: 614.

### Synthesis of Compound 19

To a stirred solution of 1-tert-butoxycarbonyl-(3S)—{N-(2-methylpropyl), N-{5-cyclopropyl-3-[3-(cis-methoxy)cyclobutyl]amino}pyridine-2-carbonyl}amino-(5R)-(morpholine-N-carbonyl)-piperidine (60 mg, 0.098 mmol, 1 eq) in dioxane (1 mL) were added a solution of HCl in dioxane (1 mL) at room temperature. The resulting mixture was stirred for 1h at room temperature under nitrogen atmosphere. The residue was neutralized to pH 8 with saturated Na₂CO₃ (aq.). The residue was purified by reverse flash chromatography with the following conditions: column, silica gel; mobile phase, MeCN in water, 0% to 60% gradient in 20 min, UV 254 nm. This resulted in Compound 19 (39.0 mg, 77.67%) as a white solid. LCMS (ESI) [M+H]⁺: 514. ¹H NMR (300 MHz, DMSO-d6) δ 7.63 (s, 1H), 6.57 (s, 1H), 5.46 (d, J=24.3, 7.0 Hz, 1H), 3.60 (m, 12H), 3.21 (m, 2H), 3.13 (s, 3H), 3.00-2.80 (m, 2H), 2.73 (d, J=6.7, 3.2 Hz, 3H), 2.60-2.53 (m, 1H), 2.45-2.21 (m, 1H), 2.07 (m, 1H), 1.87 (m, 3H), 1.59 (m, 2H), 1.00 (d, J=6.4, 4.1 Hz, 2H), 0.89 (d, J=6.7 Hz, 4H), 0.81-0.71 (m, 2H), 0.61 (d, J=6.4 Hz, 2H).

### Example 12. (3S)-[N-(2-methylpropyl), N-(5-cyclopropyl-3-cyclobutylamino)pyridine-2-carbonyl ]amino-(5R)-(morpholine-N-carbonyl)-piperidine (Compound 19, also termed as 3-(cyclobutylamino) -5-cyclopropyl-N-(2-methylpropyl)-N-[5-(morpholine-4-carbonyl) piperidin-3-yl]pyridine-2-carboxamide)

### Synthesis of methyl-3-(cyclobutylamino)-5-cyclopropylpyridine-2-carboxylate

To a stirred mixture of methyl 5-cyclopropyl-3-fluoropyridine-2-carboxylate (300 mg, 1.5 mmol, 1 eq) and cyclobutylamine (327.9 mg, 4.6 mmol, 3 eq) in NMP (5 mL) were added DIEA (595.9 mg, 4.6 mmol, 3 eq) at room temperature. The mixture was stirred at 140° C. for 2 h. The residue was purified by reverse flash chromatography with the following conditions: column, silica gel; mobile phase, MeCN in water, 10% to 60% gradient in 20 min; detector, UV 254 nm. This resulted in methyl 3-(cyclobutylamino)-5-cyclopropylpyridine-2-carboxylate (110 mg, 29.06%) as a yellow oil. LCMS (ESI) [M+H]⁺: 247.

### Synthesis of methyl-3-(cyclobutylamino)-5-cyclopropylpyridine-2-carboxylic acid

To a stirred mixture of methyl 3-(cyclobutylamino)-5-cyclopropylpyridine-2-carboxylate (110 mg, 0.44 mmol, 1 eq) and LiOH (53.4 mg, 2.235 mmol, 5 eq) in THF (1 mL) were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 10% to 60% gradient in 20 min; detector, UV 254 nm. This resulted in 3-(cyclobutylamino)-5-cyclopropylpyridine-2-carboxylic acid (100 mg, 96.40%) as a yellow oil. LCMS (ESI) [M+H]⁺: 233.

### Synthesis of methyl 1-tert-butoxycarbonyl-(3S)-[N-(2-methylpropyl), N-(5-cyclopropyl-3-cyclobutylamino)- pyridine-2-carbonyl]amino-piperidine-(5R)-carboxylate

To a stirred solution of 3-(cyclobutylamino)-5-cyclopropylpyridine-2-carboxylic acid (110 mg, 0.47 mmol, 1 eq), Compound A (178.68 mg, 0.569 mmol, 1.2 eq) and HATU (216.1 mg, 0.57 mmol, 1.2 eq) in DMF (3 mL) were added DIEA (183.6 mg, 1.42 mmol, 3 eq) at room temperature under. The resulting mixture was stirred for 1 h at room temperature under nitrogen atmosphere. The residue was purified by reverse flash chromatography with the following conditions: column, silica gel; mobile phase, MeCN in water, 10% to 60% gradient in 20 min; detector, UV 254 nm. This resulted in methyl 1-tert-butoxycarbonyl-(3S)—[N-(2-methylpropyl), N-(5-cyclopropyl-3-cyclobutylamino)-pyridine-2-carbonyl]amino-piperidine-(5R)-carboxylate (120 mg, 47.93%) as a yellow oil. LCMS (ESI) [M+H]⁺: 529.

### Synthesis of methyl 1-tert-butoxycarbonyl-(3S)-[N-(2-methylpropyl), N-[(5-cyclopropyl-3-cyclobutylamino)- pyridine-2-carbonyl]amino-piperidine-(5R)-carboxylic acid

To a stirred mixture of methyl 1-tert-butoxycarbonyl-(3S)—[N-(2-methylpropyl), N-(5-cyclopropyl-3-cyclobutylamino)-pyridine-2-carbonyl]amino-piperidine-(5R)-carboxylate (110 mg, 0.21 mmol, 1 eq) and LiOH (24.9 mg, 1.04 mmol, 5 eq) in THF (1 mL) were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 10% to 60% gradient in 20 min; detector, UV 254 nm. This resulted in 1-tert-butoxycarbonyl-(3S)—[N-(2-methylpropyl), N-[(5-cyclopropyl-3-cyclobutylamino)-pyridine-2-carbonyl]amino-piperidine-(5R)-carboxylic acid (98 mg, 91.52%) as a yellow oil. LCMS (ESI) [M+H]⁺: 515.

### Synthesis of 1-tert-butoxycarbonyl-(3S)-[N-(2-methylpropyl). N-(5-cyclopropyl-3-cyclobutylamino) pyridine-2-carbonyl]amino-(5R)-(morpholine-N-carbonyl)-piperidine

To a stirred solution of 1-tert-butoxycarbonyl-(3S)—[N-(2-methylpropyl), N-[(5-cyclopropyl-3-cyclobutylamino)-pyridine-2-carbonyl]amino-piperidine-(5R)-carboxylic acid (95 mg, 0.18 mmol, 1 eq), morpholine (19.3 mg, 0.22 mmol, 1.2 eq) and HATU (84.2 mg, 0.222 mmol, 1.2 eq) in DMF (3 mL) were added DIEA (71.5 mg, 0.55 mmol, 3 eq) at room temperature. The resulting mixture was stirred for 1 h at room temperature under nitrogen atmosphere. The residue was purified by reverse flash chromatography with the following conditions: column, silica gel; mobile phase, MeCN in water, 10% to 60% gradient in 20 min; detector, UV 254 nm. This resulted in 1-tert-butoxycarbonyl-(3S)—[N-(2-methylpropyl), N-(5-cyclopropyl-3-cyclobutylamino)pyridine-2-carbonyl]amino-(5R)-(morpholine-N-carbonyl)-piperidine (100 mg, 92.80%) as a yellow oil. LCMS (ESI) [M+H]⁺: 584.

### Synthesis of Compound 20

To a stirred solution of 1-tert-butoxycarbonyl-(3S)—[N-(2-methylpropyl), N-(5-cyclopropyl-3-cyclobutylamino)pyridine-2-carbonyl]amino-(5R)-(morpholine-N-carbonyl)-piperidine (100 mg, 0.17 mmol, 1 eq) in dioxane (1 mL) were added a solution of HCl in 1,4-dioxane (1 mL, 32.91 mmol, 192.13 eq) in portions at room temperature. The resulting mixture was stirred for 1 h at room temperature under nitrogen atmosphere. The resulting mixture was concentrated under vacuum. The mixture was neutralized to pH 8 with saturated Na₂CO₃ (aq.). The residue was purified by reverse flash chromatography with the following conditions: column, silica gel; mobile phase, MeCN in water, 10% to 60% gradient in 20 min; detector, UV 254 nm. This resulted in Compound 20 (63.7 mg, 76.89%) as a white solid. LCMS (ESI) [M+H]⁺: 484.40. ¹H NMR (300 MHz, DMSO-d6) δ 7.61 (s, 1H), 6.55 (s, 1H), 5.65-5.39 (m, 1H), 4.16-3.63 (m, 2H), 3.54 (s, 8H), 3.20 (d, J=8.0 Hz, 2H), 2.77 (d, J=46.4 Hz, 3H), 2.54 (s, 1H), 2.33 (d, J=6.1 Hz, 3H), 2.08 (s, 2H), 1.96-1.47 (m, 7H), 0.99 (d, J=8.1 Hz, 2H), 0.89 (d, J=6.7 Hz, 4H), 0.81-0.70 (m, 2H), 0.69-0.51 (m, 2H).

### Example 13. (3S)-[N-(2-methylpropyl), N-(5-cyclopropyl-3-cyclopropylamino)pyridine-2-carbonyl]amino-(5R)-[(3S)-methylmorpholine-N-carbonyl]-piperidine (Compound 21, also termed as )5-cyclopropyl-3-(cyclopropylamino)-N-[(5R)-5-[(3S)-3-methylmorpholine-4-carbonyl ]piperidin-3-yl]-N-(2-methylpropyl)pyridine-2-carboxamide)

### Synthesis of methyl-5-cyclopropyl-3-(cyclopropylamino)pyridine-2-carboxylate

To a stirred mixture of methyl 5-cyclopropyl-3-fluoropyridine-2-carboxylate (300 mg, 1.54 mmol, 1 equiv) and aminocyclopropane (263.2 mg, 4.61 mmol, 3 equiv) in NMP (5 mL) were added DIEA (595.9 mg, 4.61 mmol, 3 equiv) at room temperature. The mixture was stirred at 140° C. for 2h. The residue was purified by reverse flash chromatography with the following conditions: column, silica gel; mobile phase, MeCN in water, 10% to 60% gradient in 20 min; detector, UV 254 nm. This resulted in methyl 5-cyclopropyl-3-(cyclopropylamino)pyridine-2-carboxylate (120 mg, 33.61%) as a yellow oil. LCMS (ESI) [M+H]⁺: 233.

### Synthesis of methyl-5-cyclopropyl-3-(cyclopropylamino)pyridine-2-carboxylic acid

To a stirred mixture of methyl 5-cyclopropyl-3-(cyclopropylamino)pyridine-2-carboxylate (120 mg, 0.52 mmol, 1 eq) and LiOH (61.8 mg, 2.58 mmol, 5 eq) in THF (1 mL) were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 10% to 60% gradient in 20 min; detector, UV 254 nm. This resulted in 5-cyclopropyl-3-(cyclopropylamino)pyridine-2-carboxylic acid (102 mg, 90.46%) as a yellow oil. LCMS (ESI) [M+H]⁺: 219.

### Synthesis of methyl 1-tert-butoxycarbonyl-(3S)-[N-(2-methylpropyl), N-(5-cyclopropyl-3-cyclobutylamino) -pyridine-2-carbonyl]amino-piperidine-(5R)-carboxylate

To a stirred solution of 5-cyclopropyl-3-(cyclopropylamino)pyridine-2-carboxylic acid (105 mg, 0.48 mmol, 1 eq), Compound A (181.5 mg, 0.57 mmol, 1.2 eq) and HATU (219.5 mg, 0.57 mmol, 1.2 eq) in DMF (3 mL) were added DIEA (186.5 mg, 1.44 mmol, 3 eq) at room temperature. The resulting mixture was stirred for 1 h at room temperature under nitrogen atmosphere. The residue was purified by reverse flash chromatography with the following conditions: column, silica gel; mobile phase, MeCN in water, 10% to 70% gradient in 20 min; detector, UV 254 nm. This resulted in methyl 1-tert-butoxycarbonyl-(3S)—[N-(2-methylpropyl), N-(5-cyclopropyl-3-cyclopropylamino)pyridine-2-carbonyl]amino-piperidine-(5R)-carboxylate (120 mg, 48.47%) as a yellow oil. LCMS (ESI) [M+H]⁺: 515.

### Synthesis of methyl 1-tert-butoxycarbonyl-(3S)-[N-(2-methylpropyl), N-(5-cyclopropyl-3-cyclobutylamino) -pyridine-2-carbonyl]amino-piperidine-(5R)-carboxylic acid

To a stirred mixture of methyl 1-tert-butoxycarbonyl-(3S)—[N-(2-methylpropyl), N-(5-cyclopropyl-3-cyclopropylamino)pyridine-2-carbonyl]amino-piperidine-(5R)-carboxylate (110 mg, 0.21 mmol, 1 eq) and LiOH (25.5 mg, 1.07 mmol, 5 eq) in THF (1 mL) were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 10% to 60% gradient in 20 min; detector, UV 254 nm. This resulted in 1-tert-butoxycarbonyl-(3S)—[N-(2-methylpropyl), N-(5-cyclopropyl-3-cyclopropylamino)pyridine-2-carbonyl]amino-piperidine-(5R)-carboxylic acid (90 mg, 84.11%) as a yellow oil. LCMS (ESI) [M+H]⁺: 501.

### Synthesis of 1-tert-Butoxycarbonyl-(3S)-[N-(2-methylpropyl), N-(5-cyclopropyl-3-cyclopropylamino) pyridine-2-carbonyl]amino-(5R)-[(3S)-methylmorpholine-N-carbonyl]-piperidine

To a stirred solution of 1-tert-butoxycarbonyl-(3S)—[N-(2-methylpropyl), N-(5-cyclopropyl-3-cyclopropylamino)pyridine-2-carbonyl]amino-piperidine-(5R)-carboxylic acid (385 mg, 0.77 mmol, 1 eq) and HATU (350.8 mg, 0.924 mmol, 1.2 eq) in DMF (7 mL) was added (3S)-3-methylmorpholine (93.3 mg, 0.924 mmol, 1.2 eq), and stirred for 1 h at room temperature under nitrogen atmosphere. After the completion of the reaction, the resulting mixture was purified by reverse flash chromatography with the following conditions: column, C18 silica gel; mobile phase, MeCN in Water (0.1% FA), 0% to 60% gradient in 20 min; UV 254 nm. This resulted in 1-tert-butoxycarbonyl-(3S)—[N-(2-methylpropyl), N-(5-cyclopropyl-3-cyclopropylamino)pyridine-2-carbonyl]amino-(5R)-[(3S)-methylmorpholine-N-carbonyl]-piperidine (433 mg, 96.48%) as a white solid. LCMS (ESI) [M+H]⁺: 584.

### Synthesis of Compound 21

To a stirred solution of 1-tert-butoxycarbonyl-(3S)—[N-(2-methylpropyl), N-(5-cyclopropyl-3-cyclopropylamino)pyridine-2-carbonyl]amino-(5R)-[(3S)-methylmorpholine-N-carbonyl]-piperidine (430 mg, 0.737 mmol, 1 eq) in DCM (5 mL) was added TFA (1 mL).

The resulting mixture was stirred for 1 h at room temperature under nitrogen atmosphere. The mixture was basified to pH 8 with saturated NaHCO₃ (aq.). The aqueous layer was extracted with CH₂Cl₂ (3×10 mL), dried over anhydrous Na₂SO₄. After filtration, the filtrate was concentrated under reduced pressure. The crude product (375 mg) was purified by Prep-HPLC with the following conditions (Column: XBridge Shield RP18 OBD Column, 19*150 mm, 5 m; Mobile Phase A: Water (10 mmol/L NH₄HCO₃), Mobile Phase B: MeOH—HPLC; Flow rate: 25 mL/min; Gradient: 45% B to 67% B in 7 min, 67% B; Wave Length: 220 nm; RT1 (min): 6.06; Number Of Runs: 0, affording Compound 21 (308.1 mg, 86.48%) as a white solid. LCMS (ESI) [M+H]⁺: 484.2. ¹H NMR (400 MHz, DMSO-d6) δ 7.64 (s, 1H), 7.02-6.92 (m, 1H), 5.73 (d, J=55.2 Hz, 1H), 4.17 (d, J=127.1 Hz, 2H), 3.82 (d, J=8.8 Hz, 1H), 3.75-3.56 (m, 2H), 3.54-3.37 (m, 2H), 3.27-3.09 (m, 3H), 2.99-2.65 (m, 3H), 2.46-1.98 (m, 4H), 1.98-1.72 (m, 3H), 1.33-0.28 (m, 17H).

### Example 14. (3S)-{N-(2-methylpropyl), N-{5-cyclopropyl-3-[3-(trans-methoxy) cyclobutyl]amino}-pyridine-2-carbonyl}amino-(5R)-(5-methyl-1,3,4-oxadiazol-2-yl) -piperidine (Compound 22, also termed as 5-cyclopropyl-N-[(5R)-5-(5-methyl-1,3,4-oxadiazol -2-yl)piperidin-3-yl]-N-(2-methylpropyl)-3-{[(1r,3r)-3-methoxycyclobutyl]amino}pyridine-2-carboxamide)

### Synthesis of 1-tert-butoxylcarbonyl-(3S)-{N-(2-methylpropyl), N-{5-cyclopropyl-3-[3-(trans-methoxy) cyclobutyl]amino}pyridine-2-carbonyl}amino-(5R)-(N′-acetylhydrazinecarbonyl)-piperidine

A solution of Compound C (100 mg, 0.184 mmol, 1.0 eq), uranio borane (70.2 mg, 0.276 mmol, 1.5 eq) and N,N-diisopropylethylamine (118.6 mg, 0.920 mmol, 5.0 eq) in DMF was stirred for 10 min at room temperature. To the above mixture was added acetohydrazide (27.2 mg, 0.368 mmol, 2.0 eq). The resulting mixture was stirred for additional 1h at room temperature. The residue was purified by reverse flash chromatography with the following conditions: column, silica gel; mobile phase, MeCN in water, 0% to 50% gradient in 20 min; UV 254 nm. This resulted in 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)—(N′-acetylhydrazinecarbonyl)-piperidine (107 mg, 97.01%) as a yellow oil. LCMS (ESI) [M+H]⁺: 601.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(5-methyl-1,3,4-oxadiazol-2-yl)-piperidine

A solution of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)—(N′-acetylhydrazinecarbonyl)-piperidine (100 mg, 0.166 mmol, 1.0 eq) and Burgess reagent (158.7 mg, 0.664 mmol, 4.0 eq) in DCE was stirred for 30 min at 120° C. under nitrogen atmosphere. The residue was purified by silica gel column chromatography, eluted with CH₂Cl₂/MeOH (10:1) to afford 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-methyl-1,3,4-oxadiazol-2-yl)-piperidine (44 mg, 45.36%) as a yellow oil. LCMS (ESI) [M+H]⁺: 583.

### Synthesis of Compound 22

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-methyl-1,3,4-oxadiazol-2-yl)-piperidine (40 mg, 0.069 mmol, 1.0 eq) in a solution of HCl in 1,4-dioxane was stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 50% gradient in 20 min; UV 254 nm. This resulted in Compound 22 (20 mg, 60.37%) as a white solid. LCMS (ESI) [M+H]⁺: 483.25. ¹H NMR (400 MHz, DMSO-d6) δ 7.64 (d, J=18.8 Hz, 1H), 6.47 (d, J=10.9 Hz, 1H), 5.68 (d, J=5.8 Hz, 1H), 4.19-3.88 (m, 2H), 3.77 (s, 1H), 3.28-3.19 (m, 3H), 3.14 (s, 3H), 3.04-2.75 (m, 4H), 2.71-2.59 (m, 2H), 2.45 (d, J=5.3 Hz, 3H), 2.37-2.18 (m, 4H), 2.03 (d, J=8.4 Hz, 3H), 1.91 (m, 1H), 1.03-0.85 (m, 6H), 0.76 (d, J=5.9 Hz, 2H), 0.62 (d, J=6.5 Hz, 2H).

### Example 15. (3S)-{N-(2-methylpropyl), N-{5-cyclopropyl-3-[3-(trans-methoxy) cyclobutyl]amino}pyridine-2-carbonyl}amino-(5R)-(5-cyclopropyl-1,3,4-oxadiazol -2-yl)-piperidine (Compound 23, also termed as 5-cyclopropyl-N-[(5R)-5-(5-cyclopropyl -1,3,4-oxadiazol-2-yl)piperidin-3-yl]-N-(2-methylpropyl)-3-{[(1r,3r)-3-methoxycyclobutyl]amino}pyridine-2-carboxamide)

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(N′-cyclopropanecarbonylhydrazinecarbonyl)-piperidine

A solution of Compound C (150 mg, 0.275 mmol, 1 eq), TBTU (132.6 mg, 0.413 mmol, 1.5 eq), N,N-diisopropylethylamine (71.2 mg, 0.550 mmol, 2 eq) and cyclopropanecarbohydrazide (55.1 mg, 0.550 mmol, 2 eq) in DMF was stirred for 2 h at room temperature. The residue was purified by reverse flash chromatography with the following conditions: column, silica gel; mobile phase, MeCN in water, 0% to 50% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—(N′-cyclopropanecarbonylhydrazinecarbonyl)-piperidine (160 mg, 92.69%) as a yellow oil. LCMS (ESI) [M+H]⁺: 627.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(5-cyclopropyl-1,3,4-oxadiazol-2-yl)-piperidine

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—(N′-cyclopropanecarbonylhydrazinecarbonyl)-piperidine (160 mg, 0.255 mmol, 1 eq) and Burgess reagent (243.3 mg, 1.020 mmol, 4 eq) in DCE was stirred for 20 min at 120° C. The residue was purified by Prep-TLC (CH₂Cl₂/MeOH 20:1) to afford 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-cyclopropyl-1,3,4-oxadiazol-2-yl)-piperidine (56 mg, 36.04%) as yellow oil. LCMS (ESI) [M+H]⁺: 609.

### Synthesis of Compound 23

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-cyclopropyl-1,3,4-oxadiazol-2-yl)-piperidine (50 mg, 0.082 mmol, 1 eq) and HCl (gas) in 1,4-dioxane (2 mL) was stirred for 2 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 0% to 70% gradient in 20 min, UV 254 nm. This resulted in Compound 23 (25 mg, 59.84%) as a white solid. LCMS (ESI) [M+H]⁺: 509.3. ¹H NMR (300 MHz, DMSO-d6) δ 7.64 (d, J=12.2 Hz, 1H), 6.48 (s, 1H), 5.79-5.35 (m, 1H), 4.20-3.66 (m, 3H), 3.23 (m, 2H), 3.14 (m, 4H), 3.06-2.71 (m, 3H), 2.62 (m, 1H), 2.42 (m, 1H), 2.36-1.50 (m, 9H), 1.16-0.81 (m, 10H), 0.76 (m, 2H), 0.62 (m, 2H).

### Example 16. (3S)-{N-(2-methylpropyl). N-{5-cyclopropyl-3-[3-(trans-methoxy) cyclobutyl]amino}pyridine-2-carbonyl}amino-(5R)-(3,3-diflurorocyclobutyl-1,3,4-oxadiazol -2-yl)-piperidine (Compound 24, also termed as)5-cyclopropyl-N-[(5R)-5-[5-(3,3-difluorocyclobutyl) -1,3,4-oxadiazol-2-yl]piperidin-3-yl]-N-(2-methylpropyl)-3-{[(1r,3r)-3-methoxycyclobutyl]amino}pyridine-2-carboxamide)

### Synthesis of 3,3-difluorocyclobutane-1-carbohydrazide

To a stirred solution of methyl 3,3-difluorocyclobutane-1-carboxylate (500 mg, 3.331 mmol, 1 eq) in MeOH were added N₂H₄·H₂O (1667.2 mg, 33.310 mmol, 10 e) at room temperature. The resulting mixture was stirred for 15 h at 80° C. The resulting mixture was concentrated under reduced pressure. This resulted in 3,3-difluorocyclobutane-1-carbohydrazide (450 mg, 90.0%) as a white solid. LCMS (ESI) [M+H]⁺: 151.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-[N′-(3,3-difluorocyclobutyl) carbonylhydrazinecarbonyl]-piperidine

A solution of Compound C (150 mg, 0.275 mmol, 1 eq), 3,3-difluorocyclobutane-1-carbohydrazide (82.6 mg, 0.550 mmol, 2 eq) and DIEA (177.9 mg, 1.375 mmol, 5 equiv) in DMF was stirred for 10 min and added TBTU (132.6 mg, 0.413 mmol, 1.5 eq) at room temperature. The resulting mixture was stirred for 1 h at room temperature. The residue was purified by reverse flash chromatography with the following conditions: column, silica gel; mobile phase, MeCN in water, 0% to 70% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—[N′-(3,3-difluorocyclobutyl)carbonylhydrazinecarbonyl]-piperidine (170 mg, 91.21%) as a yellow oil. LCMS (ESI) [M+H]⁺: 677. ¹H NMR (300 MHz, DMSO-d6) δ 10.01 (s, 2H), 7.63 (s, 1H), 6.50 (s, 1H), 5.74 (d, J=96.0 Hz, 1H), 3.89 (m, 6H), 3.16 (m, 5H), 2.92 (m, 1H), 2.84-2.59 (m, 5H), 2.30 (m, 3H), 2.13-1.77 (m, 6H), 1.34 (d, J=53.2 Hz, 9H), 1.06-0.82 (m, 6H), 0.69 (d, J=31.5 Hz, 4H). ¹⁹F NMR (282 MHz, DMSO-d6) δ−80.56 (d, J=189.7 Hz, 1F), −95.02 (d, J=189.4 Hz, 1F).

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(3,3-difluorocyclobutyl-1,3,4-oxadiazol-2-yl)-piperidine

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—[N′-(3,3-difluorocyclobutyl)carbonylhydrazinecarbonyl]-piperidine (150 mg, 0.222 mmol, 1 eq) in DCE (2 mL) were added Burgess reagent (211.2 mg, 0.888 mmol, 4 eq) at room temperature. The resulting mixture was stirred for 20 min at 120° C. The residue was purified by Prep-TLC (CH₂Cl₂/MeOH 20:1) to afford 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3,3-diflurorocyclobutyl-1,3,4-oxadiazol-2-yl)-piperidine (crude product) as a yellow oil. LCMS (ESI) [M+H]⁺: 659.

### Synthesis of Compound 24

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3,3-diflurorocyclobutyl-1,3,4-oxadiazol-2-yl)-piperidine (45 mg, 0.068 mmol, 1 equiv) in DCM (1 mL) was added with TFA (0.2 mL) and stirred for 1 h at room temperature. The resulting mixture was concentrated under vacuum. The mixture was neutralized to pH 9 with saturated NaHCO₃ (aq.). The residue was purified by reverse flash chromatography with the following conditions: column, silica gel; mobile phase, MeCN in water, 00% to 60% gradient in 20 min, UV 254 nm. This resulted in Compound 24 (16.7 mg, 43.76%) as a white solid. LCMS (ESI) [M+H]⁺: 559.3. ¹H NMR (300 MHz, DMSO-d6) δ 7.65 (d, J=12.7 Hz, 1H), 6.49 (s, 1H), 5.84-5.39 (m, 1H), 4.16-3.59 (m, 4H), 2.84-3.24 (m, 12H), 2.63 (m, 1H), 2.39-2.18 (m, 4H), 2.15-1.80 (m, 5H), 1.07-0.85 (m, 6H), 0.76 (s, 2H), 0.63 (d, J=6.2 Hz, 2H).

### Example 17. (3S)-[N-(2-methylpropyl), N-(5-cyclopropyl-3-cyclopropylamino)pyridine-2-carbonyl]amino-(5R)-(morpholine-N-carbonyl)-piperidine (Compound 25)

### Synthesis of 1-tert-butoxycarbonyl-(3S)-[N-(2-methylpropyl), N-(5-cyclopropyl-3-cyclopropylamino) pyridine-2-carbonyl]amino-(5R)-(morpholine-N-carbonyl)-piperidine

To a stirred solution of 1-tert-butoxycarbonyl-(3S)—[N-(2-methylpropyl), N-(5-cyclopropyl-3-cyclopropylamino)pyridine-2-carbonyl]amino-piperidine-(5R)-carboxylic acid (98 mg, 0.2 mmol, 1 eq), morpholine (20 mg, 0.23 mmol, 1.2 eq) and HATU (89 mg, 0.23 mmol, 1.2 eq) in DMF (2 mL) were added DIEA (76 mg, 0.6 mmol, 3 eq) at room temperature. The resulting mixture was stirred for 1 h at room temperature under nitrogen atmosphere. The residue was purified by reverse flash chromatography with the following conditions: column, silica gel; mobile phase, MeCN in water, 10% to 70% gradient in 20 min; detector, UV 254 nm. This resulted in 1-tert-butoxycarbonyl-(3S)—[N-(2-methylpropyl), N-(5-cyclopropyl-3-cyclopropylamino)pyridine-2-carbonyl]amino-(5R)-(morpholine-N-carbonyl)-piperidine (105 mg, 94%) as a yellow oil. LCMS (ESI) [M+H]⁺: 570.

### Synthesis of Compound 25

To a stirred solution of 1-tert-butoxycarbonyl-(3S)—[N-(2-methylpropyl), N-(5-cyclopropyl-3-cyclopropylamino)pyridine-2-carbonyl]amino-(5R)-(morpholine-N-carbonyl)-piperidine (100 mg, 0.2 mmol, 1 eq) in dioxane (1 mL) were added a solution of HCl in 1,4-dioxane (1 mL, 32 mmol) in portions at room temperature. The resulting mixture was stirred for 1 h at room temperature under nitrogen atmosphere. The resulting mixture was concentrated under vacuum. The mixture was basified to pH 8 with saturated Na₂CO₃ (aq.). The residue was purified by reverse flash chromatography with the following conditions: column, silica gel; mobile phase, MeCN in water, 10% to 60% gradient in 20 min; detector, UV 254 nm. This resulted in Compound 25 (77.5 mg, 94%) as a white solid. LCMS (ESI) [M+H]⁺: 470.35. ¹H NMR (300 MHz, DMSO-d6) δ 7.65 (s, 1H), 6.97 (s, 1H), 5.73 (d, J=41.0 Hz, 1H), 3.54 (s, 5H), 3.41 (s, 3H), 3.17 (d, J=7.2 Hz, 2H), 3.03-2.63 (m, 3H), 2.39 (s, 2H), 2.33-2.19 (m, 1H), 2.15-1.67 (m, 4H), 1.02 (d, J=8.0 Hz, 2H), 0.87 (d, J=6.7 Hz, 4H), 0.80-0.68 (m, 4H), 0.57 (s, 2H), 0.34 (d, J=8.5 Hz, 2H).

### Example 18. (3S)-{N-(2-methylpropyl), N-{5-cyclopropyl-3-[3-(trans-methoxy)cyclobutyl]amino}pyridine-2-carbonyl}amino-peperidine (Compound 26)

### Synthesis of 1-tert-butoxycarbonyl-(3S)-(2-methylpropylamino)piperidine

To a stirred solution of 1-tert-butoxycarbonyl-(3S)-aminopiperidine (100 mg, 0.5 mmol, 1 eq) and isobutyraldehyde (43 mg, 0.6 mmol, 1.2 eq) in MeOH were added diacetyl peroxide and sodiumboranyl acetate (317 mg, 1.5 mmol, 3 eq) at room temperature. The resulting mixture was stirred for 2 h at room temperature under nitrogen atmosphere. The resulting mixture was concentrated under vacuum. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)-(2-methylpropylamino)piperidine (120 mg, 94%) as a white solid. LCMS (ESI) [M+H]⁺: 257.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-piperidine

To a stirred solution of 1-tert-butoxycarbonyl-(3S)-(2-methylpropylamino)piperidine (80 mg, 0.31 mmol, 1 eq), Compound B (98 mg, 0.4 mmol, 1.2 eq) and DIEA (121 mg, 0.9 mmol, 3 eq) in DMF were added HATU (142 mg, 0.4 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine (110 mg, 70%) as a yellow oil. LCMS (ESI) [M+H]⁺: 501.

### Synthesis of Compound 26

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine (100 mg, 0.2 mmol, 1 eq) in dioxane (1 mL) were added a solution of HCl in dioxane at room temperature. The resulting mixture was stirred for 1 h at room temperature under nitrogen atmosphere. The residue was basified to pH 8 with saturated Na₂CO₃ (aq.). The residue was purified by reverse flash chromatography with the following conditions: column, silica gel; mobile phase, MeCN in water, 0% to 70% gradient in 10 min; UV 254 nm. This resulted in Compound 26 (44 mg, 55%) as off-white oil. LCMS (ESI) [M+H]⁺: 401.25. ¹H NMR (400 MHz, DMSO-d6) δ 7.62 (d, J=13.8 Hz, 1H), 6.46 (s, 1H), 5.52 (s, 1H), 4.11-3.85 (m, 2H), 3.58 (s, 1H), 3.25-3.07 (m, 6H), 3.04-2.61 (m, 3H), 2.40-2.15 (m, 3H), 2.12-1.76 (m, 5H), 1.71-1.46 (m, 2H), 1.24-1.08 (m, 1H), 1.03-0.95 (m, 2H), 0.89 (d, J=6.7 Hz, 4H), 0.75 (s, 2H), 0.60 (s, 2H).

### Example 19. (3S)-{N-(2-methylpropyl). N-{5-cyclopropyl-3-[3-(trans-methoxy) cyclobutyl]amino}-pyridine-2-carbonyl}amino-(5R)-[(3S)-ethylmorpholine-N-carbonyl]-piperidine (Compound 27)

### Synthesis of 1-tert-butoxycarbonyl-(3S)-{N-(2-methylpropyl), N-{5-cyclopropyl-3-[3-(trans-methoxy) cyclobutyl]amino}-pyridine-2-carbonyl}amino-(5R)-[(3S)-ethylmorpholine-N-carbonyl]-piperidine

To a stirred solution of Compound C (90 mg, 0.2 mmol, 1.0 eq), HATU (75.3 mg, 0.19 mmol, 1.2 eq) and DIEA (64.0 mg, 0.49 mmol, 3.0 eq) in DMF was added (3S)-ethylmorpholine (23 mg, 0.2 mmol, 1.2 eq). The resulting mixture was stirred for 1 h at room temperature under nitrogen atmosphere. After completion of reaction, the residue was purified by reversed-phase flash chromatography with the following conditions: column, C18 silica gel; mobile phase, MeCN in Water (0.1% TFA), 0% to 60% gradient in 20 min; UV 254 nm to afford 1-tert-butoxycarbonyl-(3S)—{N-(2-methylpropyl), N-{5-cyclopropyl-3-[3-(trans-methoxy)cyclobutyl]amino}-pyridine-2-carbonyl}amino-(5R)-[(3S)-ethylmorpholine-N-carbonyl]-piperidine (95 mg, 90%) as a yellow solid. LCMS (ESI) [M+H]⁺: 642. ¹H NMR (400 MHz, Chloroform-d) δ 7.62 (s, 1H), 6.63-6.35 (m, 1H), 5.95 (d, J=178.4 Hz, 1H), 4.51-4.12 (m, 3H), 4.09-3.78 (m, 5H), 3.78-3.31 (m, 5H), 3.27 (s, 3H), 3.17-2.55 (m, 4H), 2.46-2.33 (m, 3H), 2.28-2.09 (m, 4H), 1.94-1.68 (m, 4H), 1.41 (d, J=41.9 Hz, 9H), 1.17-0.83 (m, 8H), 0.82-0.61 (m, 4H).

### Synthesis of Compound 27

To a stirred solution of 1-tert-butoxycarbonyl-(3S)—{N-(2-methylpropyl), N-{5-cyclopropyl-3-[3-(trans-methoxy)cyclobutyl]amino}-pyridine-2-carbonyl}amino-(5R)-[(3S)-ethylmorpholine-N-carbonyl]-piperidine (95 mg, 0.15 mmol, 1.0 eq) in DCM (2 mL) was added TFA (0.5 mL) and stirred for 20 min at room temperature under nitrogen atmosphere. After completion of reaction, the mixture was basified to pH 9 with saturated Na₂CO₃ (aq.). The aqueous layer was extracted with EtOAc (3×10 mL). The combined organic layers were dried over anhydrous Na₂SO₄. After filtration, the filtrate was concentrated under reduced pressure. The crude product was purified by Prep-HPLC with the following conditions to afford Compound 27 (37 mg, 46%) as a white solid. LCMS (ESI) [M+H]⁺: 542.35. ¹H NMR (400 MHz, DMSO-d6) δ 7.61 (d, J=10.0 Hz, 1H), 6.47 (s, 1H), 5.63-5.44 (m, 1H), 4.30-4.01 (m, 1H), 4.00-3.89 (m, 2H), 3.86-3.50 (m, 4H), 3.48-3.35 (m, 1H), 3.30-3.18 (m, 3H), 3.16 (s, 3H), 3.00-2.65 (m, 3H), 2.63-2.53 (m, 1H), 2.42-2.16 (m, 4H), 2.13-1.95 (m, 3H), 1.96-1.69 (m, 3H), 1.68-1.47 (m, 2H), 1.07-0.95 (m, 2H), 0.94-0.83 (m, 4H), 0.83-0.68 (m, 5H), 0.67-0.52 (m, 2H).

### Example 20. (3S)-{N-(2-methylpropyl), N-{5-cyclopropyl-3-[3-(trans-methoxy) cyclobutyl]amino}pyridine-2-carbonyl}amino-(5R)-(5-methyl-1,2,4-oxadiazol-3-yl) -piperidine (Compound 28)

### Synthesis of 1-tert-butoxycarbonyl-(3S)-{N-(2-methylpropyl), N-{5-cyclopropyl-3-[3-(transmethoxy) cyclobutyl]amino}pyridine-2-carbonyl}amino-piperidine-(5R)-carboxamide

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine-5-carboxylic acid (200 mg, 0.37 mmol, 1 eq), NH₄Cl (98 mg, 1.8 mmol, 5 eq) and DIEA (142 mg, 1.1 mmol, 3 eq) in DMF were added HATU (167 mg, 0.44 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine-(5R)-carboxamide (196 mg, 98%) as yellow oil. LCMS (ESI) [M+H]⁺: 544.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-cyano-piperidine

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine-(5R)-carboxamide (280 mg, 0.5 mmol, 1 eq) in pyridine (3 mL) was added phosphorus oxychloride (79 mg, 0.5 mmol, 1 eq) at 0° C. The resulting mixture was stirred for 1 h at room temperature under nitrogen atmosphere. The residue was purified by reverse flash chromatography with the following conditions: column, silica gel; mobile phase, MeCN in water, 0% to 50% gradient in 10 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-cyano-piperidine (208 mg, 76.83%) as yellow oil. LCMS (ESI) [M+H]⁺: 526.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(N-hydroxycarbamimidoyl)-piperidine

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-cyano-piperidine (200 mg, 0.38 mmol, 1 eq) and hydroxylamine hydrochloride (12 mg, 0.76 mmol, 2 eq) in EtOH was added DIEA (98 mg, 0.76 mmol, 2 eq) at room temperature under nitrogen atmosphere. The resulting mixture was stirred for 2 h at room temperature under nitrogen atmosphere. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—(N-hydroxycarbamimidoyl)-piperidine (110 mg, 52%) as a yellow solid. LCMS (ESI) [M+H]⁺: 559.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(5-methyl-1,2,4-oxadiazol-3-yl)-piperidine

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)—(N-hydroxycarbamimidoyl)-piperidine (105 mg, 0.19 mmol, 1 eq) and acetic anhydride (58 mg, 0.56 mmol, 3 eq) in ACN was added Et₃N (95 mg, 0.94 mmol, 5 eq) at room temperature. The resulting mixture was stirred for 2 h at 100° C. under nitrogen atmosphere. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-methyl-1,2,4-oxadiazol-3-yl)-piperidine (61 mg, 56%) as a yellow solid. LCMS (ESI) [M+H]⁺: 583.

### Synthesis of Compound 28

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(5-methyl-1,2,4-oxadiazol-3-yl)-piperidine (55 mg, 0.09 mmol, 1 eq) in 1,4-dioxane were added a solution of HCl in dioxane at room temperature. The resulting mixture was stirred for 1 h at room temperature under nitrogen atmosphere. The residue was basified to pH 8 with saturated Na₂CO₃ (aq.). The residue was purified by reverse flash chromatography with the following conditions: column, silica gel; mobile phase, MeCN in water, 0% to 60% gradient in 20 min; UV 254 nm. This resulted in Compound 28 (26.5 mg, 58%) as white oil. LCMS (ESI) [M+H]⁺: 483.30. ¹H NMR (300 MHz, DMSO-d6) δ 7.64 (d, J=13.2 Hz, 1H), 6.47 (s, 1H), 5.57 (d, J=35.6 Hz, 1H), 4.19-3.68 (m, 3H), 3.29-3.18 (m, 2H), 3.14 (s, 3H), 3.06-2.81 (m, 2H), 2.78-2.53 (m, 5H), 2.48-1.78 (m, 9H), 1.74-1.11 (m, 1H), 1.09-0.95 (m, 2H), 0.95-0.83 (m, 4H), 0.81-0.71 (m, 2H), 0.70-0.56 (m, 2H).

### Example 21. (3S)-{N-(2-methylpropyl), N-{5-cyclopropyl-3-[3-(trans-methoxy) cyclobutyl]amino}pyridine-2-carbonyl}amino-(5R)-(3-methyl-1,2,4-oxadiazol-5-yl)-piperidine (Compound 29)

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(3-methyl-1,2,4-oxadiazol-5-yl)piperidine

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-piperidine-5-carboxylic acid (150 mg, 0.28 mmol, 1.0 eq) and carbonyldiimidazole (67 mg, 0.4 mmol, 1.5 eq) in DMF was stirred for 10 h at room temperature. To the above mixture was added acetamide oxime (31 mg, 0.41 mmol, 1.5 eq) at room temperature. The resulting mixture was stirred for additional 15 h at room temperature and then at 100° C. for 1 h. The residue was purified by reverse flash chromatography with the following conditions: column, silica gel; mobile phase, MeCN in water, 0% to 70% gradient in 20 min, UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3-methyl-1,2,4-oxadiazol-5-yl)-piperidine (80 mg, 50%) as a yellow oil. LCMS (ESI) [M+H]⁺: 583.

### Synthesis of Compound 29

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(3-methyl-1,2,4-oxadiazol-5-yl)-piperidine (80 mg, 0.14 mmol, 1.0 eq) in 1,4-dioxane was added a solution of HCl in 1,4-dioxane (2 mL) at room temperature. The resulting mixture was stirred for 1 h at room temperature. The resulting mixture was concentrated under vacuum. The mixture was acidified to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 29 (15.5 mg, 23%) as a white solid. LCMS (ESI) [M+H]⁺: 483.30. ¹H NMR (300 MHz, DMSO-d6) δ 7.64 (d, J=13.1 Hz, 1H), 6.48 (s, 1H), 5.68 (m, 1H), 4.14-3.67 (m, 3H), 3.23 (m, 3H), 3.14 (s, 3H), 2.97 (m, 3H), 2.65 (m, 1H), 2.28 (m, 7H), 2.16-1.95 (m, 3H), 1.91-1.82 (m, 2H), 0.99 (m, 2H), 0.89 (d, J=6.6 Hz, 4H), 0.76 (s, 2H), 0.62 (s, 2H).

### Example 22. (3S)-[N-(2-methylpropyl), N-(5-cyclopropyl-3-(oxetan-3-ylamino)pyridine-2-carbonyl ]amino-(5R)-(morpholine-N-carbonyl)-piperidine (Compound 30)

### Synthesis of methyl-5-cyclopropyl-3-(oxetan-3-ylamino)pyrimidine-2-carboxylate

To a stirred solution of methyl 5-cyclopropyl-3-fluoropyridine-2-carboxylate (300 mg, 1.5 mmol, 1 eq) and oxetan-3-amine (135 mg, 1.8 mmol, 1.2 eq) in NMP were added DIEA (238 mg, 1.8 mmol, 1.2 eq) at room temperature under nitrogen atmosphere. The resulting mixture was stirred for 1 h at 120° C. under nitrogen atmosphere. The residue was purified by reverse flash chromatography with the following conditions: column, silica gel; mobile phase, MeCN in water, 0% to 60% gradient in 20 min; UV 254 nm. This resulted in methyl 5-cyclopropyl-3-(oxetan-3-ylamino)pyridine-2-carboxylate (90 mg, 24%) as a yellow oil. LCMS (ESI) [M+H]⁺: 249.

### Synthesis of methyl-5-cyclopropyl-3-(oxetan-3-ylamino)pyrimidine-2-carboxylic acid

To a stirred solution of methyl 5-cyclopropyl-3-(oxetan-3-ylamino)pyridine-2-carboxylate (85 mg, 0.34 mmol, 1 eq) and LiOH (41 mg, 1.7 mmol, 5 eq) in THE were added H₂O (0.5 mL) at room temperature. The resulting mixture was stirred for 1 h at room temperature under nitrogen atmosphere. The residue was acidified to pH 6 with citric acid. The residue was purified by reverse flash chromatography with the following conditions: column, silica gel; mobile phase, MeCN in water, 0% to 60% gradient in 20 min; UV 254 nm. This resulted in 5-cyclopropyl-3-(oxetan-3-ylamino)pyridine-2-carboxylic acid (65 mg, 81%) as yellow oil. LCMS (ESI) [M+H]⁺: 235.

### Synthesis of methyl 1-tert-butoxycarbonyl-(3S)-{N-(2-methylpropyl), N-[5-cyclopropyl-3-(oxetan-3-yl) amino]pyridine-2-carbonyl}amino-piperidine-(5R)-carboxylate

To a stirred solution of 5-cyclopropyl-3-(oxetan-3-ylamino)pyridine-2-carboxylic acid (60 mg, 0.25 mmol, 1 eq), Compound A (97 mg, 0.3 mmol, 1.2 eq) and DIEA (99 mg, 0.76 mmol, 3 eq) in DMF were added HATU (117 mg, 0.3 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in methyl 1-tert-butoxycarbonyl-(3S)—{N-(2-methylpropyl), N-[5-cyclopropyl-3-(oxetan-3-yl)amino]pyridine-2-carbonyl}amino-piperidine-(5R)-carboxylate (103 mg, 76%) as yellow oil. LCMS (ESI) [M+H]⁺: 531.

### Synthesis of 1-tert-butoxycarbonyl-(3S)-{N-(2-methylpropyl), N-[5-cyclopropyl-3-(oxetan-3-yl) amino]pyridine-2-carbonyl}amino-piperidine-(5R)-carboxylic acid (Compound D)

To a stirred solution of methyl 1-tert-butoxycarbonyl-(3S)—{N-(2-methylpropyl), N-[5-cyclopropyl-3-(oxetan-3-yl)amino]pyridine-2-carbonyl}amino-piperidine-(5R)-carboxylate (98 mg, 0.2 mmol, 1 eq) and LiOH (22 mg, 0.9 mmol, 5 eq) in THF were added H₂O (1 mL) at room temperature. The resulting mixture was stirred for 1 h at room temperature under nitrogen atmosphere. The residue was acidified to pH 6 with citric acid. The residue was purified by reverse flash chromatography with the following conditions: column, silica gel; mobile phase, MeCN in water, 0% to 60% gradient in 20 min; UV 254 nm. This resulted in Compound D (80 mg, 84%) as yellow oil. LCMS (ESI) [M+H]⁺: 517.

### Synthesis of 1-tert-butoxycarbonyl-(3S)-{N-(2-methylpropyl), N-[5-cyclopropyl-3-(oxetan-3-yl) amino]pyridine-2-carbonyl}amino-(5R)-(morpholine-N-carbonyl)-piperidine

To a stirred solution of Compound D (75 mg, 0.15 mmol, 1 eq), morpholine (15 mg, 0.2 mmol, 1.2 eq) and DIEA (56 mg, 0.44 mmol, 3 eq) in DMF were added HATU (66 mg, 0.17 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-(5R)-(morpholine-N-carbonyl)-piperidine (70 mg, 82%) as yellow oil. LCMS (ESI) [M+H]⁺: 586.

### Synthesis of Compound 30

To a stirred solution of 1-tert-butoxycarbonyl-(3S)—{N-(2-methylpropyl), N-[5-cyclopropyl-3-(oxetan-3-yl)amino]pyridine-2-carbonyl}amino-(5R)-(morpholine-N-carbonyl)-piperidine (60 mg, 0.10 mmol, 1 eq) in DCM was added TFA (0.3 mL) at room temperature under nitrogen atmosphere. The resulting mixture was stirred for 1 h at room temperature under nitrogen atmosphere. The resulting mixture was concentrated under vacuum and basified to pH 8 with saturated Na₂CO₃ (aq.). The residue was purified by reverse flash chromatography with the following conditions: column, silica gel; mobile phase, MeCN in water, 0% to 60% gradient in 20 min; UV 254 nm. This resulted in Compound 30 (26 mg, 53%) as a white solid. LCMS (ESI) [M+H]⁺: 486.3. ¹H NMR (300 MHz, DMSO-d6) δ 7.66 (s, 1H), 6.42 (s, 1H), 6.09-5.82 (m, 1H), 4.90-4.75 (m, 2H), 4.71-4.54 (m, 1H), 4.44-4.30 (m, 2H), 3.71-3.38 (m, 8H), 3.28-3.16 (m, 2H), 3.05-2.52 (m, 4H), 2.42-2.20 (m, 2H), 2.18-1.49 (m, 5H), 1.06-0.82 (m, 6H), 0.76 (d, 2H), 0.61 (d, J=6.4 Hz, 2H).

### Example 23. (3S)-{N-(2-methylpropyl), N-[3-(3-trans-methoxy)cyclobutyllamino-pyridine-2-carbonyl}amino-(5R)-(5-cyclopropyl-1,3,4-oxadiazol-2-yl)-piperidine (Compound 31)

### Synthesis of methyl 3-(3-trans-methoxy)cyclobutyl-amino-pyridine-2-carboxylate

To a stirred solution of methyl 3-bromopyridine-2-carboxylate (300 mg, 1.4 mmol, 1 eq), 3-trans-methoxycyclobutan-1-amine (154.5 mg, 1.53 mmol, 1.1 eq), XantPhos (161 mg, 0.28 mmol, 0.2 eq) and Cs₂CO₃ (1357 mg, 4.17 mmol, 3 eq) in dioxane was added Pd₂dba₃ (127 mg, 0.14 mmol, 0.1 eq) at room temperature. The resulting mixture was stirred for 1 h at room temperature under nitrogen atmosphere. The precipitated solids were collected by filtration and washed with CH₂Cl₂ (3×10 mL). The resulting mixture was concentrated under reduced pressure and dissolved in DMSO. The residue was purified by reverse flash chromatography with the following conditions: column, silica gel; mobile phase, MeCN in water, 0% to 50% gradient in 10 min; UV 254 nm. This resulted in methyl 3-(3-(trans-methoxy)cyclobutyl-amino-pyridine-2-carboxylate (115 mg, 35%) as a yellow oil. LCMS (ESI) [M+H]⁺: 237.

### Synthesis of methyl 3-[(3-trans-methoxy)cyclobutyl]-amino-pyridine-2-carboxylic acid

To a stirred solution of methyl 3-(3-(trans-methoxy)cyclobutyl-amino-pyridine-2-carboxylate (110 mg, 0.47 mmol, 1 eq) and LiOH (556 mg, 2.3 mmol, 5 eq) in THF were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 0% to 40% gradient in 20 min; UV 254 nm. This resulted in 3-[(3-trans-methoxy)cyclobutyl]amino-pyridine-2-carboxylic acid (98 mg, 95%) as a yellow oil. LCMS (ESI) [M+H]⁺: 223.

### Synthesis of methyl 1-tert-butoxylcarbonyl-(3S)-{N-(2-methylpropyl), N-{3-[3-(trans-methoxy) cyclobutyl]amino}pyridine-2-carbonyl}amino-piperidine-(5R)-carboxylate

To a solution of 3-[(3-trans-methoxy)cyclobutyl]amino-pyridine-2-carboxylic acid (130 mg, 0.59 mmol, 1 eq), Compound A (184 mg, 0.59 mmol, 1 eq) and DIEA (227 mg, 1.76 mmol, 3 eq) in DMF (2 mL) was added HATU (267 mg, 0.7 mmol, 1.2 eq). The mixture was stirred at room temperature for 1 h. The mixture was purified by reverse flash chromatography with the following conditions: column, silica gel; mobile phase, MeCN in water, 40% to 80% gradient in 20 min; UV 254 nm. This resulted in methyl 1-tert-butoxylcarbonyl-(3S)—{N-(2-methylpropyl), N-{3-[3-(trans-methoxy)cyclobutyl]amino}pyridine-2-carbonyl}amino-piperidine-(5R)-carboxylate (150 mg, 50%) as a yellow oil. LCMS (ESI) [M+H]⁺: 519.

### Synthesis of 1-tert-butoxylcarbonyl-(3S)-{N-(2-methylpropyl), N-{3-[3-(trans-methoxy) cyclobutyl]amino}pyridine-2-carbonyl}amino-piperidine-(5R)-carboxylic acid

To a stirred solution of methyl 1-tert-butoxylcarbonyl-(3S)—{N-(2-methylpropyl), N-{3-[3-(trans-methoxy)cyclobutyl]amino}pyridine-2-carbonyl}amino-piperidine-(5R)-carboxylate (120 mg, 0.23 mmol, 1 eq) and LiOH (28 mg, 1.2 mmol, 5 eq) in THF were added H₂O (1 mL) at room temperature. The resulting mixture was stirred for 1 h at room temperature under nitrogen atmosphere. The mixture was acidified to pH 6 with citric acid. The residue was purified by reverse flash chromatography with the following conditions: column, silica gel; mobile phase, MeCN in water, 0% to 40% gradient in 20 min; UV 254 nm. This resulted in 1-tert-butoxylcarbonyl-(3S)—{N-(2-methylpropyl), N-{3-[3-(trans-methoxy)cyclobutyl]amino}pyridine-2-carbonyl}amino-piperidine-(5R)-carboxylic acid (105 mg, 90%) as a yellow oil. LCMS (ESI) [M+H]⁺: 505.

### Synthesis of 1-tert-butoxycarbonyl-(3S)-{N-(2-methylpropyl), N-{3-[3-(trans-methoxy) cyclobutyl]amino}pyridine-2-carbonyl}amino-(5R)-(N′-cyclopropanecarbonylhydrazinecarbonyl)-piperidine

To a stirred solution of 1-tert-butoxylcarbonyl-(3S)—{N-(2-methylpropyl), N-{3-[3-(trans-methoxy)cyclobutyl]amino}pyridine-2-carbonyl}amino-piperidine-(5R)-carboxylic acid (115 mg, 0.23 mmol, 1 eq), DIEA (88.3 mg, 0.68 mmol, 3 eq) and TBTU (88 mg, 0.27 mmol, 1.2 eq) in DMF were added cyclopropanecarbohydrazide (27.3 mg, 0.27 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)—{N-(2-methylpropyl), N-{3-[3-(trans-methoxy)cyclobutyl]amino}pyridine-2-carbonyl}amino-(5R)—(N′-cyclopropanecarbonylhydrazinecarbonyl)-piperidine (121 mg, 90%) as a yellow oil. LCMS (ESI) [M+H]⁺: 587.

### Synthesis of 1-tert-butoxycarbonyl-(3S)-{N-(2-methylpropyl), N-{3-[3-(trans-methoxy) cyclobutyl]amino}pyridine-2-carbonyl}amino-(5R)-(5-cyclopropyl-1,3,4-oxadiazol-2-yl)-piperidine

To a stirred solution of I₂ (104 mg, 0.41 mmol, 2 eq) and PPh₃ (107 mg, 0.41 mmol, 2 eq) in DCE was added TEA (83 mg, 0.82 mmol, 4 eq) and 1-tert-butoxycarbonyl-(3S)—{N-(2-methylpropyl), N-{3-[3-(trans-methoxy)cyclobutyl]amino}pyridine-2-carbonyl}amino-(5R)—(N′-cyclopropanecarbonylhydrazinecarbonyl)-piperidine (120 mg, 0.21 mmol, 1 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)—{N-(2-methylpropyl), N-{3-[3-(trans-methoxy)cyclobutyl]amino}pyridine-2-carbonyl}amino-(5R)-(5-cyclopropyl-1,3,4-oxadiazol-2-yl)-piperidine (100 mg, 86%) as a yellow oil. LCMS (ESI) [M+H]⁺: 569.

### Synthesis of Compound 31

To a stirred solution of 1-tert-butoxycarbonyl-(3S)—{N-(2-methylpropyl), N-{3-[3-(trans-methoxy)cyclobutyl]amino}pyridine-2-carbonyl}amino-(5R)-(5-cyclopropyl-1,3,4-oxadiazol-2-yl)-piperidine (100 mg, 0.18 mmol, 1 eq) in DCM (0.5 mL) was added TFA (0.5 mL) at room temperature. The resulting mixture was stirred for 1 h at room temperature under nitrogen atmosphere. The mixture was basified to pH 8 with saturated Na₂CO₃ (aq.). The resulting mixture was concentrated under vacuum. The precipitated solids were collected by filtration and washed with DMSO (2×2 mL). The crude product (100 mg) was purified by Prep-HPLC with the following conditions (Column: XBridge Shield RP18 OBD Column, 19*150 mm, 5 m; Mobile Phase A: Water (10 mmol/L NH₄HCO₃), Mobile Phase B: ACN; Flow rate: 25 mL/min; Gradient: 17% B to 47% B in 7 min, 47% B; Wave Length: 220 nm; RT1 (min): 6.07; Number Of Runs: 0) to afford Compound 31 (43 mg, 52%) as a white solid. LCMS (ESI) [M+H]⁺: 469.30. 1NMR (300 MHz, DMSO-d6) δ 7.95-7.73 (m, 1H), 7.33-7.13 (m, 1H), 7.00-6.82 (m, 1H), 5.68-5.37 (m, 1H), 4.28-3.61 (m, 3H), 3.29-3.09 (m, 7H), 3.09-2.87 (m, 2H), 2.85-2.57 (m, 1H), 2.46 (s, 1H), 2.38-1.99 (m, 7H), 1.99-1.47 (m, 1H), 1.21-1.04 (m, 2H), 1.04-0.77 (m, 6H), 0.62 (d, J=6.6 Hz, 2H).

### Example 24. (3S)-{N-(2-methylpropyl), N-[5-cyclopropyl-3-(oxetan-3-yl)amino]pyridine-2-carbonyl}amino-(5R)-(5-cyclopropyl-1,3,4-oxadiazol-2-yl)-piperidine (Compound 32)

### Synthesis of 1-tert-butoxycarbonyl-(3S)-{N-(2-methylpropyl), N-[5-cyclopropyl-3-(oxetan-3-yl) amino]pyridine-2-carbonyl}amino-(5R)-(N′-cyclopropanecarbonylhydrazinecarbonyl)-piperidine

To a stirred solution of Compound D (160 mg, 0.31 mmol, 1 eq), cyclopropanecarbohydrazide (62 mg, 0.62 mmol, 2 eq) and DIEA (120 mg, 0.93 mmol, 3 eq) in DMF was added TBTU (119 mg, 0.37 mmol, 1.2 eq) at room temperature. The resulting mixture was stirred for 2 h at room temperature under nitrogen atmosphere. The residue was purified by reverse flash chromatography with the following conditions: column, silica gel; mobile phase, MeCN in water, 0% to 60% gradient in 10 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-(5R)—(N′-cyclopropanecarbonylhydrazinecarbonyl)-piperidine (150 mg, 80%) as a yellow oil. LCMS (ESI) [M+H]⁺: 599.

### Synthesis of 1-tert-butoxycarbonyl (3S)-{N-(2-methylpropyl), N-[5-cyclopropyl-3-(oxetan-3-yl) amino]pyridine-2-carbonyl}amino-(5R)-(5-cyclopropyl-1,3,4-oxadiazol-2-yl)-piperidine

To a stirred solution of 1-tert-butoxycarbonyl-(3S)—{N-(2-methylpropyl), N-[5-cyclopropyl-3-(oxetan-3-yl)amino]pyridine-2-carbonyl}amino-(5R)—(N′-cyclopropanecarbonylhydrazinecarbonyl)-piperidine (150 mg, 0.25 mmol, 1 eq) in DCE was added Burgess reagent (239 mg, 1.0 mmol, 4 eq) at room temperature. The resulting mixture was stirred for 20 min at 120° C. under nitrogen atmosphere. The residue was purified by reverse flash chromatography with the following conditions: column, silica gel; mobile phase, MeCN in water, 0% to 60% gradient in 10 min; UV 254 nm. This resulted in 1-tert-butoxycarbonyl (3S)—{N-(2-methylpropyl), N-[5-cyclopropyl-3-(oxetan-3-yl)amino]pyridine-2-carbonyl}amino-(5R)-(5-cyclopropyl-1,3,4-oxadiazol-2-yl)-piperidine (98 mg, 67%) as a yellow oil. LCMS (ESI) [M+H]⁺: 581.

### Synthesis of Compound 32

To a stirred solution of 1-tert-butoxycarbonyl (3S)—{N-(2-methylpropyl), N-[5-cyclopropyl-3-(oxetan-3-yl)amino]pyridine-2-carbonyl}amino-(5R)-(5-cyclopropyl-1,3,4-oxadiazol-2-yl)-piperidine (75 mg, 0.13 mmol, 1 eq) in DCM (0.5 mL) was added TFA (0.5 mL) at room temperature under nitrogen atmosphere. The resulting mixture was stirred for 1 h at room temperature under nitrogen atmosphere. The resulting mixture was concentrated under vacuum and basified to pH 8 with saturated Na₂CO₃ (aq.). The residue was purified by reverse flash chromatography with the following conditions: column, silica gel; mobile phase, MeCN in water, 0% to 60% gradient in 20 min; UV 254 nm. This resulted in Compound 32 (35 mg, 57%) as a white solid. LCMS (ESI) [M+H]+: 481.25. 1H NMR (300 MHz, DMSO-d6) δ 7.67 (d, J=13.0 Hz, 1H), 6.44 (d, J=7.8 Hz, 1H), 6.20-5.87 (m, 1H), 4.95-4.73 (m, 2H), 4.73-4.54 (m, 1H), 4.46-4.32 (m, 2H), 4.27-3.70 (m, 1H), 3.24 (d, 3H), 3.12-2.59 (m, 4H), 2.38-2.02 (m, 3H), 2.00-1.76 (m, 2H), 1.17-1.06 (m, 2H), 1.04-0.84 (m, 8H), 0.82-0.71 (m, 2H), 0.70-0.57 (m, 2H).

### Example 25. (3S)-{N-(2-methylpropyl), N-{6-cyclopropyl-4-[3-(trans-methoxy) cyclobutyl]amino}pyridazine-3-carbonyl}amino-(5R)-[(3S)-3-methylmorpholine-4-carbonyl]-piperidine (Compound 33)

### Synthesis of methyl 4-chloro-6-cyclopropylpyridazine-3-carboxylate

To a stirred solution of methyl 4,6-dichloropyridazine-3-carboxylate (3 g, 14.5 mmol, 1.0 eq), Pd(PPh₃)₄ (0.79 g, 0.68 mmol, 0.1 eq), cyclopropyltrifluoro-lambda4-borane potassium (2.14 g, 14.49 mmol, 1.0 eq) and H₂O (10 mL) in dioxane (30 mL) was added K₂CO₃ (6.01 g, 43.47 mmol, 3.0 eq) at room temperature under nitrogen atmosphere. The resulting mixture was stirred for 2 h at 85° C. under nitrogen atmosphere. The resulting mixture was extracted with EtOAc (100 mL). The combined organic layers were washed with water (3×100 mL), dried over anhydrous Na₂SO₄. After filtration, the filtrate was concentrated under reduced pressure and purified by silica gel column chromatography, eluted with PE/EA (1:1) to afford methyl 4-chloro-6-cyclopropylpyridazine-3-carboxylate (310 mg, 10%) as a yellow oil. LCMS (ESI) [M+H]⁺: 212. ¹H NMR (400 MHz, Chloroform-d) δ 7.01 (s, 1H), 4.06 (s, 3H), 2.61-2.45 (m, 1H), 1.41-1.20 (m, 2H), 0.98-0.81 (m, 2H).

### Synthesis of methyl 6-cyclopropyl-4-[3-(trans-methoxy)cyclobutyl]amino-pyridazine-3-carboxylate

To a stirred solution of methyl 4-chloro-6-cyclopropylpyridazine-3-carboxylate (300 mg, 1.4 mmol, 1.0 eq) in anhydrous NMP (5 mL) was added DIEA (547 mg, 4.2 mmol, 3.0 eq) and (1R,3R)-3-methoxycyclobutan-1-amine hydrochloride (233 mg, 1.7 mmol, 1.2 eq) at room temperature under nitrogen atmosphere. The reaction mixture was stirred at 140° C. for 2 h. After completion of reaction, the residue was purified by reversed-phase flash chromatography with the following conditions: column, C18 silica gel; mobile phase, MeCN in Water (0.1% TFA), 0% to 50% gradient in 20 min; UV 254 nm to afford methyl 6-cyclopropyl-4-[3-(trans-methoxy)cyclobutyl]amino-pyridazine-3-carboxylate (140 mg, 36%) as a yellow oil. LCMS (ESI) [M+H]⁺: 278.

### Synthesis of 6-cyclopropyl-4-[3-(trans-methoxy)cyclobutyl]amino-pyridazine-3-carboxylic acid

To a stirred solution of methyl 6-cyclopropyl-4-[3-(trans-methoxy)cyclobutyl]amino-pyridazine-3-carboxylate (120 mg, 0.43 mmol, 1.0 eq) and H₂O (1 mL) in THE (2 mL) was added lithium hydroxide (52 mg, 2.2 mmol, 5.0 eq) at room temperature under nitrogen atmosphere. The resulting mixture was stirred for 2 h at room temperature under nitrogen atmosphere. After completion of reaction, the mixture was acidified to pH 6 with citric acid. The residue was purified by reversed-phase flash chromatography with the following conditions: column, C18 silica gel; mobile phase, MeCN in Water (0.1% FA), 0% to 60% gradient in 20 min; UV 254 nm to afford 6-cyclopropyl-4-[3-(trans-methoxy)cyclobutyl]amino-pyridazine-3-carboxylic acid (100 mg, 88%) as a yellow oil. LCMS (ESI) [M+H]⁺: 264. ¹H NMR (400 MHz, DMSO-d6) δ 9.55 (s, 1H), 6.59 (s, 1H), 4.23-4.10 (m, 1H), 4.08-3.96 (m, 1H), 3.17 (s, 3H), 2.48-2.38 (m, 2H), 2.24-2.09 (m, 3H), 1.16-1.07 (m, 4H).

### Synthesis of methyl (3S)-{N-(2-methylpropyl), N-{6-cyclopropyl-4-[3-(trans-methoxy) cyclobutyl]amino}pyridazine-3-carbonyl}amino-piperidine-(5R)-carboxylate

To a stirred solution of 6-cyclopropyl-4-[3-(trans-methoxy)cyclobutyl]amino-pyridazine-3-carboxylic acid (100 mg, 0.38 mmol, 1.0 eq), DIEA (147.2 mg, 1.14 mmol, 3.0 eq) and HATU (173.3 mg, 0.45 mmol, 1.2 eq) in DMF (2 mL) was added Compound A (119 mg, 0.38 mmol, 1.0 eq) at room temperature under nitrogen atmosphere. The resulting mixture was stirred for 1 h at room temperature under nitrogen atmosphere. After completion of reaction, the residue was purified by reversed-phase flash chromatography with the following conditions: column, C18 silica gel; mobile phase, MeCN in Water (0.1% FA), 0% to 60% gradient in 10 min; UV 254 nm to afford methyl (3 S)—{N-(2-methylpropyl), N-{6-cyclopropyl-4-[3-(trans-methoxy)cyclobutyl]amino}pyridazine-3-carbonyl}amino-piperidine-(5R)-carboxylate (110 mg, 52%) as a yellow oil. LCMS (ESI) [M+H]⁺: 560.

### Synthesis of (3S)-{N-(2-methylpropyl), N-{6-cyclopropyl-4-[3-(trans-methoxy) cyclobutyl]amino}pyridazine-3-carbonyl}amino-piperidine-(5R)-carboxylic acid

To a stirred solution of methyl (3S)—{N-(2-methylpropyl), N-{6-cyclopropyl-4-[3-(trans-methoxy)cyclobutyl]amino}pyridazine-3-carbonyl}amino-piperidine-(5R)-carboxylate (110 mg, 0.19 mmol, 1.0 eq) and H₂O (1 mL) in THF (2 mL) was added lithium hydroxide (23.3 mg, 0.98 mmol, 5.0 eq) at room temperature under nitrogen atmosphere. The resulting mixture was stirred for 2 h at room temperature under nitrogen atmosphere. After completion of reaction, the mixture was acidified to pH 6 with citric acid. The residue was purified by reversed-phase flash chromatography with the following conditions: column, C18 silica gel; mobile phase, MeCN in Water (0.1% TFA), 0% to 60% gradient in 20 min; UV 254 nm to afford (3S)—{N-(2-methylpropyl), N-{6-cyclopropyl-4-[3-(trans-methoxy)cyclobutyl]amino}pyridazine-3-carbonyl}amino-piperidine-(5R)-carboxylic acid (95 mg, 89%) as a yellow oil. LCMS (ESI) [M+H]⁺: 546.

### Synthesis of 1-tert-butoxycarbonyl-(3S)-{N-(2-methylpropyl), N-{6-cyclopropyl-4-[3-(trans-methoxy) cyclobutyl]amino}pyridazine-3-carbonyl}amino-(5R)-[(3S)-methylmorpholine-4-carbonyl]-piperidine

To a stirred solution of (3S)—{N-(2-methylpropyl), N-{6-cyclopropyl-4-[3-(trans-methoxy)cyclobutyl]amino}pyridazine-3-carbonyl}amino-piperidine-(5R)-carboxylic acid (95 mg, 0.17 mmol, 1.0 eq), DIEA (68 mg, 0.5 mmol, 3.0 eq) and HATU (79 mg, 0.21 mmol, 1.2 eq) in DMF (2 mL) was added (3 S)-3-methylmorpholine (21 mg, 0.2 mmol, 1.2 eq) at room temperature under nitrogen atmosphere. The resulting mixture was stirred for 1 h at room temperature under nitrogen atmosphere. After completion of reaction, the residue was purified by reversed-phase flash chromatography with the following conditions: column, C18 silica gel; mobile phase, MeCN in Water (0.1% TFA), 0% to 60% gradient in 20 min; UV 254 nm to afford 1-tert-butoxycarbonyl-(3S)—{N-(2-methylpropyl), N-{6-cyclopropyl-4-[3-(trans-methoxy)cyclobutyl]amino}pyridazine-3-carbonyl}amino-(5R)-[(3S)-methylmorpholine-4-carbonyl]-piperidine (90 mg, 82%) as a yellow oil. LCMS (ESI) [M+H]⁺: 629.

### Synthesis of Compound 33

To a stirred solution of 1-tert-butoxycarbonyl-(3S)—{N-(2-methylpropyl), N-{6-cyclopropyl-4-[3-(trans-methoxy)cyclobutyl]amino}pyridazine-3-carbonyl}amino-(5R)-[(3S)-methylmorpholine-4-carbonyl]-piperidine (90 mg, 0.14 mmol, 1.0 eq) in DCM (2 mL) was added TFA (0.5 mL). The resulting mixture was stirred for 20 min at room temperature under nitrogen atmosphere. The mixture was basified to pH 9 with saturated Na₂CO₃ (aq.). The aqueous layer was extracted with EtOAc (3×10 mL). The resulting mixture was concentrated under reduced pressure. The crude product was purified by Prep-HPLC with the following conditions to afford Compound 33 (33.3 mg, 44%) as a white solid. LCMS (ESI) [M+H]⁺: 529.30. ¹H NMR (400 MHz, DMSO-d6) δ 6.47-6.41 (m, 1H), 6.39-6.10 (m, 1H), 4.42-4.13 (m, 1H), 4.08-3.99 (m, 1H), 3.98-3.90 (m, 1H), 3.89-3.72 (m, 1H), 3.66-3.38 (m, 3H), 3.29-3.17 (m, 3H), 3.14 (s, 3H), 3.04-2.78 (m, 2H), 2.77-2.65 (m, 1H), 2.63-2.54 (m, 1H), 2.48-2.41 (m, 1H), 2.40-2.28 (m, 3H), 2.27-2.02 (m, 5H), 1.99-1.52 (m, 2H), 1.33-0.87 (m, 12H), 0.62 (d, J=6.5 Hz, 2H).

### Example 26. (3S)-{N-(2-methylpropyl), N-{5-cyclopropyl-3-[3-(trans-methoxy) cyclobutyl]amino}pyridine-2-carbony}amino-(5R)-[5-(oxetan-3-yl)-1,3,4-oxadiazol-2-y1]-piperidine (Compound 34)

### Synthesis of oxetane-3-carbohydrazide

A solution of methyl oxetane-3-carboxylate (1 g, 8.6 mmol, 1.0 eq) and diimine hydrate hydrogen (3.4 g, 69 mmol, 8.0 eq) in MeOH was stirred for 48 h at room temperature. The resulting mixture was concentrated under vacuum. The residue was purified by trituration with EtOAc (10 mL). This resulted in oxetane-3-carbohydrazide (800 mg, crude) as a yellow solid. LCMS (ESI) [M+H]⁺: 117.

### Synthesis of 1-tert-butoxylcarbonyl-(3S)-{N-(2-methylpropyl), N-{5-cyclopropyl-3-[3-(trans- methoxy)cyclobutyl]amino}pyridine-2-carbonyl}amino-(5R)-(2-(oxetane-3-carbonyl)hydrazine-1-carbonyl)-piperidine

A solution of Compound C (530 mg, 0.97 mmol, 1.0 eq), DIEA (377 mg, 2.92 mmol, 3.0 eq) and TBTU (468 mg, 1.46 mmol, 1.5 eq) in DMF was stirred for 10 min at room temperature. To the above mixture was added oxetane-3-carbohydrazide (452 mg, 3.9 mmol, 4.0 eq) at room temperature. The resulting mixture was stirred for additional 4 h at room temperature. The residue was purified by reverse flash chromatography with the following conditions: column, silica gel; mobile phase, MeCN in water, 0% to 60% gradient in 20 min, UV 254 nm. This resulted in 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(2-(oxetane-3-carbonyl)hydrazine-1-carbonyl)-piperidine (310 mg, 50%) as off-white solid. LCMS (ESI) [M+H]⁺: 643. ¹H NMR (300 MHz, DMSO-d6) δ 10.01 (s, 1H), 9.87 (s, 1H), 7.64 (s, 1H), 6.50 (s, 1H), 5.76 (m, 1H), 4.75-4.48 (m, 3H), 4.10-3.66 (m, 7H), 3.15 (s, 3H), 2.82 (m, 3H), 2.31 (m, 4H), 2.16-1.84 (m, 6H), 1.55-1.13 (m, 9H), 1.00 (d, J=8.2 Hz, 2H), 0.90 (d, J=6.4 Hz, 4H), 0.75 (s, 2H), 0.64 (s, 2H).

### Synthesis of 1-tert-butoxylcarbonyl-(3S)-{N-(2-methylpropyl), N-{5-cyclopropyl-3-[3-(trans-methoxy) cyclobutyl]amino}pyridine-2-carbonyl}amino-(5R)-[5-(oxetane-3-yl)-1,3,4-oxadiazol-2-yl)-piperidine

A solution of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(2-(oxetane-3-carbonyl)hydrazine-1-carbonyl)-piperidine (160 mg, 0.25 mmol, 1.0 eq) and Burgess reagent (237 mg, 1.0 mmol, 4.0 eq) in DCE was stirred for 20 min at 120° C. under nitrogen atmosphere. The residue was purified by reverse flash chromatography with the following conditions: column, silica gel; mobile phase, MeCN in water, 0% to 60% gradient in 20 min, UV 254 nm. This resulted in 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-[5-(oxetane-3-yl)-1,3,4-oxadiazol-2-yl)-piperidine (45 mg, 29%) as off-white solid. LCMS (ESI) [M+H]⁺: 625.

### Synthesis of Compound 34

A solution of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-[5-(oxetane-3-yl)-1,3,4-oxadiazol-2-yl)-piperidine (130 mg, 0.21 mmol, 1.0 eq) in DCM/TFA was stirred for 30 min at room temperature. The mixture was acidified to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 34 (49 mg, 45%) as a white solid. LCMS (ESI) [M+H]⁺: 525.30. ¹H NMR (300 MHz, DMSO-d6) δ 7.65 (d, J=13.7 Hz, 1H), 6.49 (s, 1H), 5.82-5.45 (m, 1H), 4.90 (m, 2H), 4.75 (m, 2H), 4.54 (d, J=8.2 Hz, 1H), 4.18-3.66 (m, 3H), 3.24 (m, 3H), 3.14 (s, 3H), 3.10-2.79 (m, 3H), 2.72-2.57 (m, 1H), 2.29 (m, 4H), 1.98 (m, 5H), 1.06-0.97 (m, 2H), 0.90 (d, J=6.6 Hz, 4H), 0.76 (s, 2H), 0.63 (d, J=6.4 Hz, 2H).

### Example 27 (3S)-{N-(2-methylpropyl), N-{5-cyclopropyl-3-[3-(trans-methoxy) cyclobutyl]amino}pyridine-2-carbonyl}amino-(5R)-hydroxymethyl-piperidine (Compound 35)

A solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-hydroxymethyl-piperidine (70 mg, 0.13 mmol, 1.0 eq) in DCM/TFA was stirred for 30 min at room temperature. The mixture was acidified to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 34 (32.3 mg, 57%) as a white solid. LCMS (ESI) [M+H]⁺: 431.25. ¹H NMR (300 MHz, DMSO-d6) δ 7.63 (d, J=9.1 Hz, 1H), 6.48 (s, 1H), 5.52 (d, J=5.9 Hz, 1H), 4.41 (s, 1H), 3.79 (d, J=97.8 Hz, 3H), 3.15 (m, 7H), 3.03-2.64 (m, 3H), 2.43 (m, 1H), 2.30 (m, 2H), 2.15-1.74 (m, 6H), 1.53 (m, 1H), 1.42-1.16 (m, 1H), 1.05-0.95 (m, 2H), 0.89 (d, J=6.6 Hz, 4H), 0.76 (s, 2H), 0.60 (d, J=6.3 Hz, 2H).

### Example 28. (3S)-{N-(2-methylpropyl), N-[5-cyclopropyl-3-(oxetan-3-yl)amino]pyridine-2-carbonyl}amino-piperidine (Compound 36)

### Synthesis of 1-tert-butoxycarbonyl-(3S)-{N-(2-methylpropyl), N-[5-cyclopropyl-3-(oxetan-3-yl) amino]pyridine-2-carbonyl}amino-piperidine

To a stirred solution of 1-tert-butoxycarbonyl-(3S)-(2-methylpropylamino)piperidine (80 mg, 0.31 mmol, 1 eq), 5-cyclopropyl-3-(oxetan-3-ylamino)pyridine-2-carboxylic acid (88 mg, 0.37 mmol, 1.2 eq) and DIEA (121 mg, 0.94 mmol, 3 eq) in DMF (1 mL) were added HATU (142 mg, 0.37 mmol, 1.2 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-piperidine (100 mg, 68%) as a yellow solid. LCMS (ESI) [M+H]⁺: 473.

### Synthesis of Compound 36

To a stirred solution of 1-tert-butoxycarbonyl-(3S)—{N-(2-methylpropyl), N-[5-cyclopropyl-3-(oxetan-3-yl)amino]pyridine-2-carbonyl}amino-piperidine (100 mg, 0.212 mmol, 1 eq) in DCM (0.5 mL) was added TFA (0.5 mL) at room temperature. The resulting mixture was stirred for 1 h at room temperature under nitrogen atmosphere. The mixture was basified to pH 8 with saturated NaHCO₃ (aq.). The crude product (100 mg) was purified by Prep-HPLC with the following conditions (Column: XBridge Shield RP18 OBD Column, 19*150 mm, 5 m; Mobile Phase A: Water (10 mmol/L NH₄HCO₃), Mobile Phase B: ACN; Flow rate: 25 mL/min; Gradient: 19% B to 45% B in 7 min, 45% B; Wave Length: 220 nm; RT1 (min): 5.57; Number Of Runs: 0) to afford Compound 36 (33 mg, 41%) as an off-white oil. LCMS (ESI) [M+H]⁺: 373.20. ¹H NMR (300 MHz, DMSO-d6) δ 7.66 (d, J=9.8 Hz, 1H), 6.43 (d, J=7.2 Hz, 1H), 5.94 (d, J=6.2 Hz, 1H), 4.91-4.77 (m, 2H), 4.72-4.57 (m, 1H), 4.42-4.30 (m, 2H), 3.61-3.47 (m, 1H), 3.21 (d, J=7.5 Hz, 3H), 3.04-2.54 (m, 3H), 2.44-1.95 (m, 2H), 1.94-1.36 (m, 5H), 1.03-0.85 (m, 6H), 0.81-0.72 (m, 2H), 0.61 (d, J=6.5 Hz, 2H)

### Example 29. (3S){N-(2-methylpropyl), N-[5-cyclopropyl-3-(oxetan-3-yl)amino]-pyridine-2-carbonyl}amino-(5R)-(5-methyl-1,3,4-oxadiazol-2-yl)-piperidine (Compound 37)

### Synthesis of 1-tert-butoxycarbonyl-(3S)-{N-(2-methylpropyl), N-[5-cyclopropyl-3-(oxetan-3-yl) amino]pyridine-2-carbonyl}amino-(5R)-( N′-acetylhydrazinecarbonyl)-piperidine

To a stirred solution of Compound D (210 mg, 0.41 mmol, 1 eq), TBTU (157 mg, 0.5 mmol, 1.2 eq) and DIEA (158 mg, 1.2 mmol, 3 eq) in DMF was added acetohydrazide (36 mg, 0.5 mmol, 1.2 eq) at room temperature. The resulting mixture was stirred for 1 h at room temperature under nitrogen atmosphere. The residue was purified by reverse flash chromatography with the following conditions: column, C18 silica gel; mobile phase, MeCN in Water (0.1% TFA), 0% to 70% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-(5R)—(N′-acetylhydrazinecarbonyl)-piperidine (200 mg, 86%) as a yellow oil. ¹H NMR (400 MHz, DMSO-d6) δ 9.98-9.70 (m, 2H), 7.66 (s, 1H), 6.46 (d, J=21.2 Hz, 1H), 6.11 (d, J=95.6 Hz, 1H), 4.88-4.75 (m, 2H), 4.70-4.56 (m, 1H), 4.47-4.32 (m, 2H), 4.20-3.56 (m, 4H), 3.25-3.08 (m, 1H), 3.06-2.58 (m, 2H), 2.41-2.04 (m, 2H), 2.03-1.78 (m, 6H), 1.49-1.20 (m, 9H), 1.04-0.57 (m, 10H). LCMS (ESI) [M+H]⁺: 573.

### Synthesis of 1-tert-butoxycarbonyl (3S)-{N-(2-methylpropyl), N-[5-cyclopropyl-3-(oxetan-3-yl) amino]pyridine-2-carbony}amino-(5R)-(5-methyl-1,3,4-oxadiazol-2-yl)-piperidine

A solution of PPh₃ (164.8 mg, 0.628 mmol, 2 eq) in DCM was treated with I₂ (159.5 mg, 0.63 mmol, 2 eq) for 10 min at room temperature under nitrogen atmosphere followed by the addition of TEA (127 mg, 1.3 mmol, 4 eq) and 1-tert-butoxycarbonyl-(3S)—{N-(2-methylpropyl), N-[5-cyclopropyl-3-(oxetan-3-yl)amino]pyridine-2-carbonyl}amino-(5R)—(N′-acetylhydrazinecarbonyl)-piperidine (180 mg, 0.31 mmol, 1 eq). The resulting mixture was stirred for additional 1 h at room temperature. The reaction was quenched with NaSO₃ at room temperature. The residue was purified by reverse flash chromatography with the following conditions: column, C18 silica gel; mobile phase, MeCN in Water (0.1% TFA), 0% to 60% gradient in 20 min; UV 254 nm. This resulted in 1-tert-butoxycarbonyl (3S)—{N-(2-methylpropyl), N-[5-cyclopropyl-3-(oxetan-3-yl)amino]pyridine-2-carbonyl}amino-(5R)-(5-methyl-1,3,4-oxadiazol-2-yl)-piperidine (80 mg, 46%) as a yellow oil. ¹H NMR (400 MHz, DMSO-d6) δ 7.72-7.60 (m, 1H), 6.46 (d, J=17.1 Hz, 1H), 6.12 (d, J=111.4 Hz, 1H), 4.89-4.78 (m, 2H), 4.70-4.57 (m, 1H), 4.46-4.33 (m, 2H), 4.31-3.47 (m, 4H), 3.26-3.12 (m, 2H), 3.11-2.75 (m, 2H), 2.49-2.41 (m, 3H), 2.36-2.21 (m, 1H), 2.19-2.02 (m, 1H), 1.95-1.62 (m, 2H), 1.51-1.21 (m, 9H), 1.03-0.60 (m, 10H). LCMS (ESI) [M+H]⁺: 555.

### Synthesis of Compound 37

To a stirred solution of 1-tert-butoxycarbonyl (3S)—{N-(2-methylpropyl), N-[5-cyclopropyl-3-(oxetan-3-yl)amino]pyridine-2-carbonyl}amino-(5R)-(5-methyl-1,3,4-oxadiazol-2-yl)-piperidine (65 mg, 0.12 mmol, 1 eq) in CH₂Cl₂ was added TFA (0.3 mL) at room temperature. The resulting mixture was stirred for 30 min at room temperature under nitrogen atmosphere. The mixture was basified to pH 8 with saturated NaHCO₃ (aq.). The crude product (65 mg) was purified by Prep-HPLC with the following conditions (Column: XBridge Prep OBD C18 Column, 30*150 mm, 5 m; Mobile Phase A: Water (10 mmol/L NH4HCO3), Mobile Phase B: ACN; Flow rate: 60 mL/min; Gradient: 21% B to 45% B in 7 min, 45% B; Wave Length: 220 nm; RT1 (min): 5.65; Number Of Runs: 0) to afford Compound 37 (24.3 mg, 46%) as a white solid. LCMS (ESI) [M+H]⁺: 455.25. ¹H NMR (300 MHz, DMSO-d6) δ 7.67 (d, J=13.8 Hz, 1H), 6.44 (d, J=8.3 Hz, 1H), 6.16-5.88 (m, 1H), 4.92-4.76 (m, 2H), 4.72-4.54 (m, 1H), 4.46-4.32 (m, 2H), 3.92 (d, J=104.8 Hz, 1H), 3.28-3.20 (m, 2H), 3.19-3.08 (m, 1H), 3.07-2.73 (m, 3H), 2.71-2.53 (m, 1H), 2.48-2.36 (m, 4H), 2.33-2.17 (m, 2H), 1.99-1.50 (m, 2H), 1.04-0.55 (m, 10H).

### Example 30. (3S)-{N-(2-methylpropyl), N-[5-cyclopropyl-3-(oxetan-3-yl)amino]-pyridine-2-carbonyl}amino-(5R)-(3-methyl-1,2,4-oxadiazol-5-yl)-piperidine (Compound 38)

### Synthesis of 1-tert-butoxycarbonyl-(3S)-{N-(2-methylpropyl), N-[5-cyclopropyl-3-(oxetan-3-yl) amino]-pyridine-2-carbonyl}amino-(5R)-(3-methyl-1,2,4-oxadiazol-5-yl)-piperidine

A solution of Compound D (150.0 mg, 0.29 mmol, 1.0 eq) and carbonyldiimidazole (141 mg, 0.87 mmol, 3.0 eq) in DMF was stirred for 5 h at room temperature under nitrogen atmosphere. To the above mixture was added acetamide oxime (64.5 mg, 0.87 mmol, 3.0 eq) at room temperature. The resulting mixture was stirred for additional 15 h at room temperature and then 100° C. for 1 h. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-(2-methylpropyl), N-[5-cyclopropyl-3-(oxetan-3-yl)amino]-pyridine-2-carbonyl}amino-(5R)-(3-methyl-1,2,4-oxadiazol-5-yl)-piperidine (80.0 mg, 50%) as a yellow oil. LCMS (ESI) [M+H]⁺: 555.

### Synthesis of Compound 38

A solution of 1-tert-butoxycarbonyl-(3S)—{N-(2-methylpropyl), N-[5-cyclopropyl-3-(oxetan-3-yl)amino]-pyridine-2-carbonyl}amino-(5R)-(3-methyl-1,2,4-oxadiazol-5-yl)-piperidine (60.0 mg, 0.11 mmol, 1.0 eq) in DCM/TFA (1.5:0.5 mL) was stirred for 0.5 h at room temperature. The mixture was acidified to pH 9 with saturated NaHCO₃ (aq.). The residue was purified by reverse flash chromatography with the following conditions: column, silica gel; mobile phase, MeCN in water, 0% to 50% gradient in 20 min; UV 254 nm. This resulted in Compound 38 (21.6 mg, 43.93%) as a white solid. LCMS (ESI) [M+H]⁺: 455.30. ¹H NMR (400 MHz, DMSO-d₆) δ 7.67 (d, J=19.5 Hz, 1H), 6.44 (d, J=13.0 Hz, 1H), 6.01 (m, 1H), 4.83 (m, 2H), 4.63 (m, 1H), 4.37 (m, 2H), 3.76 (d, J=11.6 Hz, 1H), 3.75-3.71 (m, 1H), 3.23 (m, 3H), 3.07-2.88 (m, 3H), 2.73-2.57 (m, 1H), 2.46 (m, 1H), 2.31 (d, 4H), 2.18-1.85 (m, 2H), 1.14-0.50 (m, 10H).

### Example 31. (3S)-{N-(2-methylpropyl), N-{5-cyclopropyl-3-[3-(trans-methoxy) cyclobutyl]amino }pyridine-2-carbonyl }amino-(5R)-(1,2,4-oxadiazol-3-yl)-piperidine (Compound 39)

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(1,2,4-oxadiazol-3-yl)-piperidine

To a stirred solution of 1-tert-butoxycarbonyl-(3S)—{N-(2-methylpropyl), N-{5-cyclopropyl-3-[(1R,3R)-3-methoxycyclobutyl]amino}pyridine-2-carbonyl}amino-(5R)—(N-hydroxycarbamimidoyl)-piperidine (110 mg, 0.2 mmol, 1 eq) in triethyl orthoformate (1 mL) was added BF₃·Et₂O (2.7 mg, 0.02 mmol, 0.1 eq) at room temperature under nitrogen atmosphere. The resulting mixture was stirred for 1 h at 100° C. under nitrogen atmosphere. The residue was purified by reverse flash chromatography with the following conditions: column, silica gel; mobile phase, MeCN in water, 0% to 60% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(1,2,4-oxadiazol-3-yl)-piperidine (95 mg, 85%) as a yellow oil. LCMS (ESI) [M+H]⁺: 569.

### Synthesis of Compound 39

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(1,2,4-oxadiazol-3-yl)-piperidine (80 mg, 0.14 mmol, 1 eq) in DCM (0.4 mL) was added TFA (0.4 mL) at room temperature. The resulting mixture was stirred for 1 h at room temperature under nitrogen atmosphere. The mixture was basified to pH 8 with saturated NaHCO₃ (aq.). The crude product (80 mg) was purified by Prep-HPLC with the following conditions (Column: XBridge Prep C18 OBD Column, 30*50 mm, 5 m 13 nm; Mobile Phase A: Water (10 mmol/L NH₄HCO₃), Mobile Phase B: ACN; Flow rate: 60 mL/min; Gradient: 22% B to 45% B in 8 min, 45% B; Wave Length: 220 nm; RT1 (min): 8.4; Number Of Runs: 0) to afford Compound 39 (32.6 mg, 49%) as a white solid. LCMS (ESI) [M+H]⁺: 469.20. ¹H NMR (400 MHz, DMSO-d6) δ 9.51 (d, J=5.3 Hz, 1H), 7.64 (d, J=19.8 Hz, 1H), 6.47 (d, J=8.8 Hz, 1H), 5.73-5.46 (m, 1H), 4.20-3.71 (m, 3H), 3.31-3.19 (m, 2H), 3.19-3.05 (m, 4H), 3.04-2.93 (m, 1H), 2.91-2.83 (m, 1H), 2.78 (s, 1H), 2.70-2.54 (m, 1H), 2.47-2.36 (m, 1H), 2.36-2.25 (m, 2H), 2.25-2.16 (m, 1H), 2.13-1.96 (m, 3H), 1.96-1.79 (m, 2H), 1.05-0.95 (m, 2H), 0.95-0.81 (m, 4H), 0.81-0.70 (m, 2H), 0.70-0.56 (m, 2H).

### Example 32. (3S){N-(2-methylpropyl), N-{5-cyclopropyl-3-[3-(trans-methoxy) cyclobutyl]amino}-pyridine-2-carbonyl}amino-(5R)-(1,3,4-oxadiazol-2-yl)-piperidine (Compound 40)

### Synthesis of 1-tert-butoxylcarbonyl-(3S)-{N-(2-methylpropyl), N-{5-cyclopropyl-3-[3-(trans-methoxy) cyclobutyl]amino}pyridine-2-carbonyl}amino-(5R)-(hydrazinecarbonyl)-piperidine

To a stirred solution of Compound C (200 mg, 0.37 mmol, 1 eq), HATU (168 mg, 0.44 mmol, 1.2 eq) and DIEA (142 mg, 1.1 mmol, 3 eq) in DMF (3 mL) were added diimine hydrate hydrogen (90.0 mg, 1.8 mmol, 5 eq) at room temperature. The resulting mixture was stirred for 1 h at room temperature under nitrogen atmosphere. The residue was purified by reverse flash chromatography with the following conditions: column, silica gel; mobile phase, MeCN in water, 0% to 70% gradient in 20 min; UV 254 nm. This resulted in 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(hydrazinecarbonyl)-piperidine (175 mg, 85%) as a yellow oil. LCMS (ESI) [M+H]⁺: 559.

### Synthesis of 1-tert-butoxycarbonyl-(3S)-{N-(2-methylpropyl), N-{5-cyclopropyl-3-[3-(trans-methoxy) cyclobutyl]amino}pyridine-2-carbonyl}amino-(5R)-(1,3,4-oxadiazol-2-yl)-piperidine

To a stirred solution of 1-tert-butoxylcarbonyl-(3S)—{N-(2-methylpropyl), N-{5-cyclopropyl-3-[3-(trans-methoxy)cyclobutyl]amino}pyridine-2-carbonyl}amino-(5R)-(hydrazinecarbonyl)-piperidine (150 mg, 0.27 mmol, 1 eq) in triethyl orthoformate (2 mL) was added Boron trifluoride etherate (0.4 mg, 0.003 mmol, 0.01 eq) at room temperature. The resulting mixture was stirred for 1 h at 90° C. under nitrogen atmosphere. The resulting mixture was concentrated under reduced pressure. The residue was purified by reverse flash chromatography with the following conditions: column, silica gel; mobile phase, MeCN in water, 0% to 50% gradient in 20 min; UV 254 nm. This resulted in 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(1,3,4-oxadiazol-2-yl)-piperidine (80 mg, 52%) as a yellow oil. LCMS (ESI) [M+H]⁺: 569.

### Synthesis of Compound 40

To a stirred solution of 1-tert-butoxycarbonyl-(3S)—{N-(2-methylpropyl), N-{5-cyclopropyl-3-[3-(trans-methoxy)cyclobutyl]amino}pyridine-2-carbonyl}amino-(5R)-(1,3,4-oxadiazol-2-yl)-piperidine (60 mg, 0.11 mmol, 1 eq) in DCM (0.5 mL) was added TFA (0.5 mL) at room temperature. The resulting mixture was stirred for 1 h at room temperature under nitrogen atmosphere. The mixture was basified to pH 8 with saturated NaHCO₃ (aq.). The resulting mixture was concentrated under reduced pressure. The crude product (60 mg) was purified by Prep-HPLC with the following conditions (Column: XBridge Shield RP18 OBD Column, 19*150 mm, 5 m; Mobile Phase A: Water (10 mmol/L NH₄HCO₃), Mobile Phase B: ACN; Flow rate: 25 mL/min; Gradient: 26% B to 47% B in 7 min, 47% B; Wave Length: 220 nm; RT1 (min): 6.11; Number Of Runs: 0) to afford Compound 40 (25.3 mg, 51%) as a white solid. LCMS (ESI) [M+H]⁺: 469.35. ¹H NMR (400 MHz, DMSO-d6) δ 9.13 (s, 1H), 7.64 (d, J=18.3 Hz, 1H), 6.48 (s, 1H), 5.59 (d, J=64.8 Hz, 1H), 4.17-3.87 (m, 3H), 3.86-3.71 (m, 1H), 3.27-3.17 (m, 3H), 3.14 (s, 3H), 3.11-2.84 (m, 3H), 2.67-2.59 (m, 1H), 2.36-2.18 (m, 4H), 2.12-1.86 (m, 4H), 1.03-0.81 (m, 6H), 0.79-0.70 (m, 2H), 0.67-0.57 (m, 2H).

### Example 33. (3S)-{N-(2-cyclopropylmethyl, N-{5-cyclopropyl-3-[3-(trans-methoxy) cyclobutyl]amino}-pyridine-2-carbonyl}amino-(5R)-(5-methyl-1,3,4-oxadiazol-2-yl)-piperidine (Compound 41)

### Synthesis of methyl 1-tert-butoxylcarbonyl-(3S)-(2-cyclopropylmethyl)amino-piperidine (5R)-carboxylate

A solution of methyl 1-tert-butoxylcarbonyl (3S)-amino-piperidine-(5R)-carboxylate (600.0 mg, 2.3 mmol, 1.0 eq), cyclopropanecarbaldehyde (195 mg, 2.8 mmol, 1.2 eq) and sodium triacetoxyborohydride (1477 mg, 7 mmol, 3.0 eq) in MeOH was stirred for 2 h at room temperature. The residue was purified by reverse flash chromatography with the following conditions: column, silica gel; mobile phase, MeCN in water, 0% to 70% gradient in 20 min, UV 254 nm. This resulted in methyl 1-tert-butoxylcarbonyl-(3S)-(2-cyclopropylmethyl)amino-piperidine (5R)-carboxylate (560 mg, 77%) as a yellow oil. LCMS (ESI) [M+H]⁺: 313.

### Synthesis of methyl 1-tert-butoxylcarbonyl-(3S)-{N-cyclopropylmethyl, N-{5-cyclopropyl-3-[3-(trans-methoxy)cyclobutyl]amino}-pyridine-2-carbonyl}amino-piperidine-(5R)-carboxylate

A solution of methyl 1-tert-butoxylcarbonyl-(3S)-(2-cyclopropylmethyl)amino-piperidine (5R)-carboxylate (180 mg, 0.57 mmol), Compound B (150 mg, 0.57 mmol, 1.0 eq), N,N-diisopropylethylamine (222 mg, 1.7 mmol, 3.0 eq) and HATU (261 mg, 0.7 mmol, 1.2 eq) in DMF was stirred for 1 h at room temperature. The residue was purified by reverse flash chromatography with the following conditions: column, silica gel; mobile phase, MeCN in water, 0% to 60% gradient in 20 min, UV 254 nm. This resulted in methyl 1-tert-butoxylcarbonyl-(3S)—{N-cyclopropylmethyl, N-{5-cyclopropyl-3-[3-(trans-methoxy)cyclobutyl]amino}-pyridine-2-carbonyl}amino-piperidine-(5R)-carboxylate (260 mg, 82%) as a yellow oil. LCMS (ESI) [M+H]⁺: 557. ¹H NMR (400 MHz, Chloroform-d) δ 7.65 (d, J=1.8 Hz, 1H), 6.42 (s, 1H), 5.90 (d, J=81.7 Hz, 1H), 4.28 (s, 2H), 4.02 (m, 3H), 3.70 (s, 3H), 3.27 (s, 3H), 2.66 (m, 3H), 2.40 (m, 4H), 2.15 (m, 2H), 1.83 (m, 2H), 1.73 (s, 1H), 1.44 (s, 9H), 1.14-0.93 (m, 3H), 0.72 (m, 2H), 0.49 (m, 4H).

### Synthesis of 1-tert-butoxylcarbonyl-(3S)-{N-cyclopropylmethyl, N-{5-cyclopropyl-3-[3-(trans-methoxy) cyclobutyl]amino}-pyridine-2-carbonyl}amino-piperidine-(5R)-carboxylic acid

A solution of methyl 1-tert-butoxylcarbonyl-(3S)—{N-cyclopropylmethyl, N-{5-cyclopropyl-3-[3-(trans-methoxy)cyclobutyl]amino}pyridine-2-carbonyl}amino-piperidine-(5R)-carboxylate (260.0 mg, 0.47 mmol, 1.0 eq) and lithium hydrate (98 mg, 2.34 mmol, 5.0 eq) in THF/H₂O (8:4 mL) was stirred for 2 h at room temperature. The mixture was acidified to pH 4 with citric acid. The residue was purified by reverse flash chromatography with the following conditions: column, silica gel; mobile phase, MeCN in water, 0% to 50% gradient in 20 min, UV 254 nm. This resulted in 1-tert-butoxylcarbonyl-(3S)—{N-cyclopropylmethyl, N-{5-cyclopropyl-3-[3-(trans-methoxy)cyclobutyl]amino}-pyridine-2-carbonyl}amino-piperidine-(5R)-carboxylic acid (240 mg, 95%) as a yellow solid. LCMS (ESI) [M+H]⁺: 543. ¹H NMR (400 MHz, DMSO-d₆) δ 12.56 (s, 1H), 7.62 (s, 1H), 6.49 (s, 1H), 5.67 (d, J=54.9 Hz, 1H), 4.37-3.63 (m, 5H), 3.31 (s, 3H), 3.14 (s, 3H), 3.03-2.58 (m, 2H), 2.31 (m, 2H), 2.06 (m, 4H), 1.89 (m, 2H), 1.33 (d, J=71.6 Hz, 9H), 0.98 (m, 2H), 0.74 (d, J=5.8 Hz, 2H), 0.56-0.17 (m, 4H).

### Synthesis of 1-tert-butoxylcarbonyl-(3S)-{N-cyclopropylmethyl, N-{5-cyclopropyl-3-[3-(trans-methoxy) cyclobutyl]amino}pyridine-2-carbonyl}amino-(5R)-(N″-acetylhydrazinecarbonyl)-piperidine

A solution of 1-tert-butoxylcarbonyl-(3S)—{N-cyclopropylmethyl, N-{5-cyclopropyl-3-[3-(trans-methoxy)cyclobutyl]amino}-pyridine-2-carbonyl}amino-piperidine-(5R)-carboxylic acid (150 mg, 0.28 mmol, 1.0 eq), N,N-diisopropylethylamine (178.6 mg, 1.38 mmol, 5.0 eq) and TBTU (133.1 mg, 0.41 mmol, 1.5 eq) in DMF was stirred for 10 min at room temperature. To the above mixture was added acetohydrazide (30.7 mg, 0.41 mmol, 1.5 eq) at room temperature. The resulting mixture was stirred for additional 1 h at room temperature. The residue was purified by reverse flash chromatography with the following conditions: column, silica gel; mobile phase, MeCN in water, 0% to 50% gradient in 20 min, UV 254 nm. This resulted in 1-tert-butoxylcarbonyl-(3S)—{N-cyclopropylmethyl, N-{5-cyclopropyl-3-[3-(trans-methoxy)cyclobutyl]amino}pyridine-2-carbonyl}amino-(5R)—(N′-acetylhydrazinecarbonyl)-piperidine (130.0 mg, 79%) as a yellow oil. LCMS (ESI) [M+H]⁺: 599. ¹H NMR (300 MHz, DMSO-d₆) δ 9.89 (d, J=13.0 Hz, 2H), 9.78 (s, 1H), 7.63 (s, 1H), 6.50 (s, 1H), 5.71 (d, J=50.5 Hz, 1H), 4.15-3.65 (m, 5H), 3.34 (s, 2H), 3.15 (s, 3H), 3.08-2.59 (m, 2H), 2.30 (d, J=6.0 Hz, 3H), 2.03-1.89 (m, 3H), 1.84 (s, 3H), 1.35 (d, J=50.8 Hz, 9H), 1.15-0.93 (m, 3H), 0.75 (m, 3H), 0.59-0.21 (m, 4H).

### Synthesis of 1-tert-butoxycarbonyl-(3S)-{N-cyclopropylmethyl, N-{5-cyclopropyl-3-[3-(trans-methoxy) cyclobutyl]amino}pyridine-2-carbonyl}amino-(5R)-(5-methyl-1,3,4-oxadiazol-2-yl)-piperidine

A solution of iodine (110.2 mg, 0.43 mmol, 2.0 eq) and triphenylphosphine (113.9 mg, 0.43 mmol, 2.0 eq) in DCM was stirred for 10 min at room temperature under nitrogen atmosphere. To the above mixture was added triethylamine (87.9 mg, 0.87 mmol, 4.0 eq) and 1-tert-butoxylcarbonyl-(3S)—{N-cyclopropylmethyl, N-{5-cyclopropyl-3-[3-(trans-methoxy)cyclobutyl]amino}pyridine-2-carbonyl}amino-(5R)—(N′-acetylhydrazinecarbonyl)-piperidine (130 mg, 0.22 mmol, 1.0 eq) at room temperature. The resulting mixture was stirred for additional 2 h at room temperature. The resulting mixture was concentrated under vacuum. The residue was purified by reverse flash chromatography with the following conditions: column, silica gel; mobile phase, MeCN in water, 0% to 60% gradient in 20 min, UV 254 nm. This resulted in 1-tert-butoxycarbonyl-(3S)—{N-cyclopropylmethyl, N-{5-cyclopropyl-3-[3-(trans-methoxy)cyclobutyl]amino}pyridine-2-carbonyl}amino-(5R)-(5-methyl-1,3,4-oxadiazol-2-yl)-piperidine (70 mg, 56%) as a yellow oil. LCMS (ESI) [M+H]⁺: 581.

### Synthesis of Compound 41

A solution of 1-tert-butoxycarbonyl-(3S)—{N-cyclopropylmethyl, N-{5-cyclopropyl-3-[3-(trans-methoxy)cyclobutyl]amino}pyridine-2-carbonyl}amino-(5R)-(5-methyl-1,3,4-oxadiazol-2-yl)-piperidine (70.0 mg, 0.12 mmol, 1.0 eq) in DCM/TFA (1.5:0.5 mL) was stirred for 30 min at room temperature. The mixture was acidified to pH 9 with saturated NaHCO₃ (aq.). The residue was purified by reverse flash chromatography with the following conditions: column, silica gel; mobile phase, MeCN in water, 0% to 50% gradient in 20 min, UV 254 nm. This resulted in Compound 41 (42 mg, 73%) as a white solid. LCMS (ESI) [M+H]⁺: 481.30. ¹H NMR (400 MHz, DMSO-d₆) δ 7.73 (d, J=22.4 Hz, 1H), 6.57 (s, 1H), 5.85-5.53 (m, 1H), 4.37-3.78 (m, 3H), 3.43-3.29 (m, 2H), 3.23 (s, 3H), 3.16-2.87 (m, 3H), 2.80-2.63 (m, 1H), 2.60 (m, 4H), 2.46-2.28 (m, 4H), 2.21-2.09 (m, 2H), 2.00 (m, 2H), 1.26-0.77 (m, 5H), 0.56 (d, J=8.1 Hz, 1H), 0.41 (d, J=8.6 Hz, 2H), 0.00 (s, 1H).

### Example 34. (3S)-{N-(2-methylpropyl), N-[5-cyclopropyl-3-(oxetan-3-yl)amino]pyridine-2-carbonyl }amino-(5R)-(1,2,4-oxadiazol-3-yl)-piperidine (Compound 42)

### Synthesis of 1-tert-butoxycarbonyl-(3S)-{N-(2-methylpropyl), N-[5-cyclopropyl-3-(oxetan-3-yl) amino]pyridine-2-carbonyl}amino-piperidine-(5R)-carboxamide

To a stirred solution of Compound D (870 mg, 1.7 mmol, 1 eq) and DIEA (652.9 mg, 5.052 mmol, 3 eq) in DMF (10 mL) was added NH₄Cl (180.1 mg, 3.368 mmol, 2 eq) and HATU (960 mg, 2.526 mmol, 1.5 eq). The mixture was stirred for 1 h at room temperature under nitrogen atmosphere. The residue was purified by reverse flash chromatography with the following conditions: column, C18 silica gel; mobile phase, MeCN in Water (0.1% TFA), 0% to 60% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-piperidine-(5R)-carboxamide (850 mg, 98%) as a yellow solid. LCMS (ESI) [M+H]⁺: 516.

### Synthesis of 1-tert-butoxycarbonyl-(3S)-{N-(2-methylpropyl), N-[5-cyclopropyl-3-(oxetan-3-yl) amino]pyridine-2-carbonyl}amino-(5R)-cyano-piperidine

A solution of 1-tert-butoxycarbonyl-(3S)—{N-(2-methylpropyl), N-[5-cyclopropyl-3-(oxetan-3-yl)amino]pyridine-2-carbonyl}amino-piperidine-(5R)-carboxamide (850 mg, 1.648 mmol, 1 eq) and phosphorus oxychloride (252.7 mg, 1.648 mmol, 1 eq) in pyridine (8 mL) was stirred for 45 min at room temperature under nitrogen atmosphere. The reaction was quenched with water at room temperature. The residue was purified by reverse flash chromatography with the following conditions: column, C18 silica gel; mobile phase, MeCN in Water (0.1% FA), 0% to 60% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-(5R)-cyano-piperidine (405 mg, 49%) as a yellow solid. LCMS (ESI) [M+H]⁺: 498.

### Synthesis of 1-tert-butoxycarbonyl-(3S)-{N-(2-methylpropyl), N-[5-cyclopropyl-3-(oxetan-3-yl) amino]pyridine-2-carbonyl}amino-(5R)-(N-hydroxycarbamimidoyl)-piperidine

A solution of 1-tert-butoxycarbonyl-(3S)—{N-(2-methylpropyl), N-[5-cyclopropyl-3-(oxetan-3-yl)amino]pyridine-2-carbonyl}amino-(5R)-cyano-piperidine (405 mg, 0.814 mmol, 1 eq), hydroxylamine hydrochloride (113.1 mg, 1.628 mmol, 2 eq) and DIEA (210.3 mg, 1.628 mmol, 2 eq) in EtOH (5 mL) was stirred for 30 min at 70° C. under nitrogen atmosphere. The residue was purified by reverse flash chromatography with the following conditions: column, C18 silica gel; mobile phase, MeCN in Water (0.1% FA), 0% to 60% gradient in 20 min; UV 254 nm. This resulted in 1-tert-butoxycarbonyl-(3S)—{N-(2-methylpropyl), N-[5-cyclopropyl-3-(oxetan-3-yl)amino]pyridine-2-carbonyl}amino-(5R)—(N-hydroxycarbamimidoyl)piperidine (220 mg, 51%) as a yellow solid. LCMS (ESI) [M+H]⁺: 531.

### Synthesis of 1-tert-butoxycarbonyl-(3S)-{N-(2-methylpropyl), N-[5-cyclopropyl-3-(oxetan-3-yl) amino]-pyridine-2-carbonyl}amino-(5R)-(1,2,4-oxadiazol-3-yl)-piperidine

A solution of 1-tert-butoxycarbonyl-(3S)—{N-(2-methylpropyl), N-[5-cyclopropyl-3-(oxetan-3-yl)amino]pyridine-2-carbonyl}amino-(5R)—(N-hydroxycarbamimidoyl)piperidine (220 mg, 0.42 mmol, 1 eq) and 4-methylbenzene-1-sulfonic acid (7.1 mg, 0.04 mmol, 0.1 eq) in triethyl orthoformate (2.5 mL) was stirred for 30 min at 125° C. under nitrogen atmosphere. The resulting mixture was concentrated under reduced pressure. The residue was purified by reverse flash chromatography with the following conditions: column, C18 silica gel; mobile phase, MeCN in Water (0.1% TFA), 0% to 60% gradient in 20 min; UV 254 nm. This resulted in 1-tert-butoxycarbonyl-(3S)—{N-(2-methylpropyl), N-[5-cyclopropyl-3-(oxetan-3-yl)amino]-pyridine-2-carbonyl}amino-(5R)-(1,2,4-oxadiazol-3-yl)-piperidine (132 mg, 59%) as a yellow oil. LCMS (ESI) [M+H]⁺: 541.

### Synthesis of Compound 42

To a stirred solution of 1-tert-butoxycarbonyl-(3S)—{N-(2-methylpropyl), N-[5-cyclopropyl-3-(oxetan-3-yl)amino]-pyridine-2-carbonyl}amino-(5R)-(1,2,4-oxadiazol-3-yl)-piperidine (125 mg, 0.231 mmol, 1 eq) in DCM (1 mL) was added TFA (1 mL) and stirred for 15 min at room temperature under nitrogen atmosphere. The mixture was basified to pH 8 with saturated Na₂CO₃ (aq.). The crude product (125 mg) was purified by Prep-HPLC with the following conditions (Column: XBridge Prep OBD C18 Column, 30*150 mm, 5 m; Mobile Phase A: Water (10 mmol/L NH4HCO3), Mobile Phase B: ACN; Flow rate: 60 mL/min; Gradient: 18% B to 50% B in 7.5 min, 50% B; Wave Length: 220 nm; RT1 (min): 6.23; Number Of Runs: 0) to afford Compound 42 (63 mg, 62%) as a white solid. LCMS (ESI) [M+H]⁺: 441.20. ¹H NMR (300 MHz, DMSO-d6) δ 9.51 (d, J=5.2 Hz, 1H), 7.68 (d, J=14.7 Hz, 1H), 6.44 (d, J=10.6 Hz, 1H), 6.16-5.83 (m, 1H), 4.90-4.78 (m, 2H), 4.74-4.56 (m, 1H), 4.45-4.33 (m, 2H), 4.24-3.67 (m, 1H), 3.29-3.18 (m, 2H), 3.15 (d, J=13.7 Hz, 1H), 3.06-2.85 (m, 2H), 2.84-2.71 (m, 1H), 2.70-2.54 (m, 1H), 2.49-2.32 (m, 1H), 2.30-2.00 (m, 2H), 1.97-1.50 (m, 2H), 1.03-0.95 (m, 2H), 0.95-0.83 (m, 4H), 0.82-0.71 (m, 2H), 0.69-0.58 (m, 2H).

### Example 35. (3S)-{N-(2-methylpropyl), N-[5-trifluoromethyl-3-(oxetan-3-yl)amino]pyridine-2-carbonyl}amino-(5R)-(morpholine-N-carbonyl)-piperidine (Compound 43)

### Synthesis of methyl 3-(oxetan-3-ylamino)-5-(trifluoromethyl)pyridine-2-carboxylate

A solution of methyl 3-bromo-5-(trifluoromethyl)pyridine-2-carboxylate (950 mg, 3.35 mmol, 1 eq), oxetan-3-amine (293 mg, 4.0 mmol, 1.2 eq), Pd₂(dba)₃ (306 mg, 0.34 mmol, 0.1 eq), and Xantphos (387 mg, 0.67 mmol, 0.2 eq) in 1,4-dioxane was treated with Cs₂CO₃ (3269 mg, 10 mmol, 3 eq) at room temperature. The resulting mixture was stirred for 2 h at 85° C. under nitrogen atmosphere. The resulting mixture was extracted with CH₂Cl₂ (3×20 mL). The combined organic layers were washed with CH₂Cl₂ (3×300 mL), dried over anhydrous Na₂SO₄. After filtration, the filtrate was concentrated under reduced pressure. The residue was purified by silica gel column chromatography, eluted with PE/EA (5:1) to afford methyl 3-(oxetan-3-ylamino)-5-(trifluoromethyl)pyridine-2-carboxylate (330 mg, 36%) as a yellow solid. LCMS (ESI) [M+H]⁺: 277.

### Synthesis of 3-(oxetan-3-ylamino)-5-(trifluoromethyl)pyridine-2-carboxylic acid

To a stirred solution of methyl 3-(oxetan-3-ylamino)-5-(trifluoromethyl)pyridine-2-carboxylate (330 mg, 1.195 mmol, 1 eq) and H₂O (3 mL) in THE (3 mL) was added LiOH (143.0 mg, 5.975 mmol, 5 eq) at room temperature. The resulting mixture was stirred for 1 h at room temperature under nitrogen atmosphere. The residue was acidified to pH 6 with citric acid. The residue was purified by reverse flash chromatography with the following conditions: column, C18 silica gel; mobile phase, MeCN in Water (0.1% TFA), 0% to 40% gradient in 20 min; UV 254 nm. This resulted in 3-(oxetan-3-ylamino)-5-(trifluoromethyl)pyridine-2-carboxylic acid (305 mg, 97.37%) as a yellow solid. LCMS (ESI) [M+H]⁺: 263.

### Synthesis of methyl 1-tert-butoxycarbonyl-(3S)-{N-(2-methylpropyl), N-[5-trifluoromethyl-3-(oxetan -3-ylamino]pyridine-2-carbonyl}amino-piperidine-(5R)-carboxylate

To a stirred solution of 3-(oxetan-3-ylamino)-5-(trifluoromethyl)pyridine-2-carboxylic acid (100 mg, 0.38 mmol, 1 eq) and DIEA (147.8 mg, 1.143 mmol, 3 eq) in DMF (1.5 mL) was added Compound A (144 mg, 0.46 mmol, 1.2 eq) and HATU (217 mg, 0.57 mmol, 1.5 eq) at room temperature under nitrogen atmosphere. The resulting mixture was stirred for 1 h at room temperature under nitrogen atmosphere. The residue was purified by reverse flash chromatography with the following conditions: column, C18 silica gel; mobile phase, MeCN in Water (0.1% FA), 0% to 60% gradient in 20 min; UV 254 nm. This resulted in methyl 1-tert-butoxycarbonyl-(3S)—{N-(2-methylpropyl), N-[5-trifluoromethyl-3-(oxetan-3-ylamino]pyridine-2-carbonyl}amino-piperidine-(5R)-carboxylate (130 mg, 61%) as a yellow oil. LCMS (ESI) [M+H]⁺: 559.

### Synthesis of 1-tert-butoxycarbonyl-(3S)-{N-(2-methylpropyl), N-[5-trifluoromethyl-3-(oxetan-3- ylamino]pyridine-2-carbonyl}amino-piperidine-(5R)-carboxylic acid

To a stirred solution of methyl 1-tert-butoxycarbonyl-(3S)—{N-(2-methylpropyl), N-[5-trifluoromethyl-3-(oxetan-3-ylamino]pyridine-2-carbonyl}amino-piperidine-(5R)-carboxylate (120 mg, 0.22 mmol, 1 eq) and H₂O (1 mL) in THF (1 mL) was added LiOH (26 mg, 1.1 mmol, 5 eq) and stirred for 1 h at room temperature under nitrogen atmosphere. The residue was acidified to pH 6 with citric acid. The residue was purified by reverse flash chromatography with the following conditions: column, C18 silica gel; mobile phase, MeCN in Water (0.1% TFA), 0% to 40% gradient in 20 min; UV 254 nm. This resulted in 1-tert-butoxycarbonyl-(3S)—{N-(2-methylpropyl), N-[5-trifluoromethyl-3-(oxetan-3-ylamino]pyridine-2-carbonyl}amino-piperidine-(5R)-carboxylic acid (105 mg, 90%) as a yellow oil. LCMS (ESI) [M+H]⁺: 545.

### Synthesis of 1-tert-butoxycarbonyl-(3S)-{N-(2-methylpropyl), N-[5-trifluoromethyl-3-(oxetan-3-yl) amino]pyridine-2-carbonyl}amino-(5R)-(morpholine-N-carbonyl)-piperidine

To a stirred solution of 1-tert-butoxycarbonyl-(3S)—{N-(2-methylpropyl), N-[5-trifluoromethyl-3-(oxetan-3-ylamino]pyridine-2-carbonyl}amino-piperidine-(5R)-carboxylic acid (120 mg, 0.22 mmol, 1 eq) and DIEA (85.4 mg, 0.660 mmol, 3 eq) in DMF (1.5 mL) was added morpholine (23.0 mg, 0.264 mmol, 1.2 eq) and HATU (100.4 mg, 0.264 mmol, 1.2 eq), and stirred for 1 h at room temperature under nitrogen atmosphere. The residue was purified by reverse flash chromatography with the following conditions: column, C18 silica gel; mobile phase, MeCN in Water (0.1% TFA), 0% to 60% gradient in 10 min; UV 254 nm. This resulted in 1-tert-butoxycarbonyl-(3S)—{N-(2-methylpropyl), N-[5-trifluoromethyl-3-(oxetan-3-yl)amino]pyridine-2-carbonyl}amino-(5R)-(morpholine-N-carbonyl)-piperidine (110 mg, 81.34%) as a yellow oil. LCMS (ESI) [M+H]⁺: 614.

### Synthesis of Compound 43

A solution of 1-tert-butoxycarbonyl-(3S)—{N-(2-methylpropyl), N-[5-trifluoromethyl-3-(oxetan-3-yl)amino]pyridine-2-carbonyl}amino-(5R)-(morpholine-N-carbonyl)-piperidine (120 mg, 0.20 mmol, 1 eq) and TFA (1 mL) in DCM (1 mL) was stirred for 15 min at room temperature under nitrogen atmosphere. The mixture was basified to pH 8 with saturated Na₂CO₃ (aq.). The crude product (120 mg) was purified by Prep-HPLC with the following conditions (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 18% B to 50% B in 7.5 min, 50% B; Wave Length: 220 nm; RT1 (min): 6.23; Number Of Runs: 0) to afford Compound 43 (63 mg, 63%) as a white solid. LCMS (ESI) [M+H]⁺: 514.25. ¹H NMR (300 MHz, DMSO-d6) δ 8.25-8.11 (m, 1H), 7.12 (d, J=1.8 Hz, 1H), 6.52-6.25 (m, 1H), 4.88-4.80 (m, 2H), 4.80-4.66 (m, 1H), 4.52-4.42 (m, 2H), 3.66-3.36 (m, 9H), 3.30-2.65 (m, 5H), 2.63-2.54 (m, 1H), 2.47-1.99 (m, 3H), 1.97-1.55 (m, 2H), 0.93 (d, J=6.6 Hz, 4H), 0.65 (d, J=6.5 Hz, 2H).

### Example 36. (3S)-{N-cyclopropylmethyl, N-{5-cyclopropyl-3-[3-(trans-methoxy) cyclobutyl]amino}-pyridine-2-carbonyl}amino-(5R)-(1,3,4-oxadiazol-2-yl)-piperidine (Compound 44)

### Synthesis of 1-tert-butoxylcarbonyl-(3S)-{N-cyclopropylmethyl, N-{5-cyclopropyl-3-[3-(trans-methoxy) cyclobuty]amino}pyridine-2-carbonyl}amino-(5R)-(hydrazinecarbonyl)-piperidine

To a stirred solution of 1-tert-butoxycarbonyl-(3S)—{N-cyclopropylmethyl, N-{5-cyclopropyl-3-[3-(trans-methoxy)cyclobutyl]amino}pyridine-2-carbonyl}amino-piperidine-(5R)-carboxylic acid (370 mg, 0.68 mmol, 1 eq), DIEA (264 mg, 2.0 mmol, 3.00 eq) and HATU (311 mg, 0.82 mmol, 1.2 eq) in DMF (4 mL) was added diimine hydrate hydrogen (66.8 mg, 1.364 mmol, 2 eq). The resulting mixture was stirred for 2 h at room temperature under nitrogen atmosphere. The residue was purified by reverse flash chromatography with the following conditions: column, C18 silica gel; mobile phase, MeCN in Water (0.1% TFA), 0% to 60% gradient in 20 min; UV 254 nm. This resulted in 1-tert-butoxylcarbonyl-(3S)—{N-cyclopropylmethyl, N-{5-cyclopropyl-3-[3-(trans-methoxy)cyclobutyl]amino}pyridine-2-carbonyl}amino-(5R)-(hydrazinecarbonyl)-piperidine (140 mg, 37%) as a white solid. LCMS (ESI) [M+H]⁺: 557.

### Synthesis of 1-tert-butoxycarbonyl-(3S)-{N-cyclopropylmethyl, N-{5-cyclopropyl-3-[3-(trans-methoxy) cyclobutyl]amino}pyridine-2-carbonyl}amino-(5R)-(1,3,4-oxadiazol-2-yl)-piperidine

To a stirred solution of 1-tert-butoxylcarbonyl-(3S)—{N-cyclopropylmethyl, N-{5-cyclopropyl-3-[3-(trans-methoxy)cyclobutyl]amino}pyridine-2-carbonyl}amino-(5R)-(hydrazinecarbonyl)-piperidine (130 mg, 0.23 mmol, 1 eq) and intriethyl orthoformate (1.5 mL) was added 4-methylbenzene-1-sulfonic acid (4.0 mg, 0.02 mmol, 0.1 eq). The resulting mixture was stirred for 15 min at 125° C. under nitrogen atmosphere. The residue was purified by reverse flash chromatography with the following conditions: column, C18 silica gel; mobile phase, MeCN in Water (0.1% TFA), 0% to 60% gradient in 20 min; UV 254 nm. This resulted in 1-tert-butoxycarbonyl-(3S)—{N-cyclopropylmethyl, N-{5-cyclopropyl-3-[3-(trans-methoxy)cyclobutyl]amino}pyridine-2-carbonyl}amino-(5R)-(1,3,4-oxadiazol-2-yl)-piperidine (60 mg, 45%) as a white solid. LCMS (ESI) [M+H]⁺: 567.

### Synthesis of Compound 44

To a stirred solution of 1-tert-butoxycarbonyl-(3S)—{N-cyclopropylmethyl, N-{5-cyclopropyl-3-[3-(trans-methoxy)cyclobutyl]amino}pyridine-2-carbonyl}amino-(5R)-(1,3,4-oxadiazol-2-yl)-piperidine (50 mg, 0.09 mmol, 1 eq) in DCM (0.5 mL) was added TFA (0.5 mL). The resulting mixture was stirred for 15 min at room temperature under nitrogen atmosphere. The mixture was basified to pH 8 with saturated NaHCO₃ (aq.). The crude product (50 mg) was purified by Prep-HPLC with the following conditions (Column: XBridge Shield RP18 OBD Column, 19*150 mm, 5 m; Mobile Phase A: Water (10 mmol/L NH₄HCO₃), Mobile Phase B: MeOH—HPLC; Flow rate: 25 mL/min; Gradient: 43% B to 66% B in 7 min, 66% B; Wave Length: 220 nm; RT1 (min): 5.78; Number Of Runs: 0) to afford Compound 44 (19 mg, 45%) as a white solid. LCMS (ESI) [M+H]⁺: 467.15. ¹H NMR (400 MHz, DMSO-d6) δ 9.13 (s, 1H), 7.63 (d, J=20.7 Hz, 1H), 6.48 (s, 1H), 5.57 (d, J=20.9 Hz, 1H), 4.31-3.69 (m, 3H), 3.26-3.18 (m, 2H), 3.17-3.10 (m, 3H), 3.00 (d, J=54.2 Hz, 3H), 2.75-2.53 (m, 2H), 2.35-2.20 (m, 4H), 2.05 (s, 3H), 1.95-1.86 (m, 1H), 1.18-0.92 (m, 3H), 0.80 (d, J=40.0 Hz, 3H), 0.47 (d, J=7.7 Hz, 1H), 0.33 (s, 2H), −0.09 (s, 1H).

### Example 37. (3S)-{N-(2-methylpropyl). N-{5-cyclopropyl-3-[3-(trans-methoxy-d3) cyclobutyl]amino}-pyridine-2-carbonyl}amino-(5R)-(1,3,4-oxadiazol-2-yl)-piperidine (Compound 45)

### Synthesis of tert-butyl N-[3-trans-(methoxy-d3)cyclobutyl]carbamate

To a stirred solution of tert-butyl N-(3-trans-hydroxycyclobutyl)carbamate (1 g, 5.34 mmol, 1 eq) and iodo (methane-d3) (2323 mg, 16 mmol, 3 eq) in THF was added NaH (385 mg, 16 mmol, 3 eq) at 0° C. under nitrogen atmosphere. The resulting mixture was stirred for 2 h at room temperature under nitrogen atmosphere. The resulting mixture was concentrated under reduced pressure. The residue was dissolved in DMSO (5 mL). The residue was purified by reverse flash chromatography with the following conditions: column, C18 silica gel; mobile phase, MeCN in Water (0.1% FA), 0% to 60% gradient in 20 min; UV 254 nm. This resulted in tert-butyl N-[3-trans-(methoxy-d3)cyclobutyl]carbamate (520 mg, 48%) as a white solid. LCMS (ESI) [M+H]⁺: 205. ¹H NMR (400 MHz, DMSO-d6) δ 7.15 (d, 1H), 4.02-3.91 (m, 1H), 3.91-3.83 (m, 1H), 2.17-2.00 (m, 4H), 1.37 (s, 9H).

### Synthesis of 3-trans-(methoxy-d3)cyclobutan-1-amine

To a stirred solution of tert-butyl N-[3-trans-(methoxy-d3)cyclobutyl]carbamate (510 mg, 2.5 mmol, 1 eq) in dioxane (5 mL) was added a solution of HCl in 1,4-dioxane (5 mL). The resulting mixture was stirred for 1 h at room temperature under nitrogen atmosphere. The resulting mixture was concentrated under reduced pressure. The resulting mixture was filtered, the filter cake was washed with diethyl ether (3×30 mL). The filtrate was concentrated under reduced pressure. This resulted in 3-trans-(methoxy-d3)cyclobutan-1-amine (270 mg, crude) as a white solid. LCMS (ESI) [M+H]⁺: 105.

### Synthesis of methyl 5-cyclopropyl-3-[3-trans-(methoxy-d3)cyclobutyl]amino-pyridine-2-carboxylate

To a stirred solution of 3-trans-(methoxy-d3)cyclobutan-1-amine (260 mg, 2.5 mmol, 1 eq) and DIEA (968 mg, 7.5 mmol, 3 eq) in NMP was added methyl 5-cyclopropyl-3-fluoropyridine-2-carboxylate (585 mg, 3.0 mmol, 1.2 eq). The resulting mixture was stirred for 4 h at 140° C. under nitrogen atmosphere. The residue was purified by reverse flash chromatography with the following conditions: column, C18 silica gel; mobile phase, MeCN in Water (0.1% TFA), 0% to 60% gradient in 20 min; UV 254 nm. This resulted in methyl 5-cyclopropyl-3-[3-trans-(methoxy-d3)cyclobutyl]amino-pyridine-2-carboxylate (150 mg, 22%) as a yellow oil. LCMS (ESI) [M+H]⁺: 280.

### Synthesis of 5-cyclopropyl-3-[3-(trans-methoxy-d3)cyclobutyl]amino-pyridine 2-carboxylic acid

To a stirred solution of methyl 5-cyclopropyl-3-[3-trans-(methoxy-d3)cyclobutyl]amino-pyridine-2-carboxylate (140 mg, 0.50 mmol, 1 eq) and H₂O (0.5 mL) in THF (0.5 mL) was added LiOH (60 mg, 2.5 mmol, 5 eq) at room temperature. The resulting mixture was stirred for 1 h at room temperature under nitrogen atmosphere. The mixture was acidified to pH 6 with citric acid. The residue was purified by reverse flash chromatography with the following conditions: column, C18 silica gel; mobile phase, MeCN in Water (0.1% TFA), 0% to 40% gradient in 10 min; UV 254 nm. This resulted in 5-cyclopropyl-3-[3-(trans-methoxy-d3)cyclobutyl]amino-pyridine-2-carboxylic acid (110 mg, 83%) as a yellow solid. LCMS (ESI) [M+H]⁺: 266.

### Synthesis of methyl 1-tert-butoxylcarbonyl-(3S)-{N-(2-methylpropyl), N-[5-cyclopropyl-3-[3-(trans-methoxy-d3) cyclobutyl]amino]pyridine-2-carbonyl}amino-piperidine-(5R)-carboxylate

To a stirred solution of 5-cyclopropyl-3-[3-(trans-methoxy-d3)cyclobutyl]amino-pyridine-2-carboxylic acid (110 mg, 0.42 mmol, 1 eq), HATU (189.1 mg, 0.498 mmol, 1.2 eq) and DIEA (160.7 mg, 1.245 mmol, 3 eq) in DMF (1.5 mL) was added Compound A (156 mg, 0.5 mmol, 1.2 eq) at room temperature. The resulting mixture was stirred for 1 h at room temperature under nitrogen atmosphere. The residue was purified by reverse flash chromatography with the following conditions: column, C18 silica gel; mobile phase, MeCN in Water (0.1% TFA), 0% to 60% gradient in 20 min; UV 254 nm. This resulted in methyl 1-tert-butoxylcarbonyl-(3S)—{N-(2-methylpropyl), N-{5-cyclopropyl-3-[3-(trans-methoxy-d3)cyclobutyl]amino}-pyridine-2-carbonyl}amino-piperidine-(5R)-carboxylate (180 mg, 77%) as a yellow oil. LCMS (ESI) [M+H]⁺: 562.

### Synthesis of 1-tert-butoxylcarbonyl-(3S)-{N-(2-methylpropyl), N-{5-cyclopropyl-3-[3-(trans-methoxy-d3) cyclobutyl]amino}pyridine-2-carbonyl}amino-piperidine-(5R)-carboxylic acid

To a stirred solution of methyl 1-tert-butoxylcarbonyl-(3S)—{N-(2-methylpropyl), N-{5-cyclopropyl-3-[3-(trans-methoxy-d3)cyclobutyl]amino}pyridine-2-carbonyl}amino-piperidine-(5R)-carboxylate (170 mg, 0.3 mmol, 1 eq) and H₂O (1 mL) in THF (1 mL) was added LiOH (36 mg, 1.5 mmol, 5 eq) at room temperature. The resulting mixture was stirred for 1 h at room temperature under nitrogen atmosphere. The residue was acidified to pH 6 with citric acid. The residue was purified by reverse flash chromatography with the following conditions: column, C18 silica gel; mobile phase, MeCN in Water (0.1% FA), 0% to 40% gradient in 200 min; UV 254 nm. This resulted in 1-tert-butoxylcarbonyl-(3S)—{N-(2-methylpropyl), N-{5-cyclopropyl-3-[3-(trans-methoxy-d3)cyclobutyl]amino}pyridine-2-carbonyl}amino-piperidine-(5R)-carboxylic acid (150 mg, 90%) as a white solid. LCMS (ESI) [M+H]⁺: 548.

### Synthesis of 1-tert-butoxylcarbonyl-(3S)-{N-(2-methylpropyl), N-{5-cyclopropyl-3-[3-(trans-methoxy-d3)cyclobutyl]amino}pyridine-2-carbonyl}amino-(5R)- (hydrazinecarbonyl)-piperidine

To a stirred solution of 1-tert-butoxylcarbonyl-(3S)—{N-(2-methylpropyl), N-{5-cyclopropyl-3-[3-(trans-methoxy-d3)cyclobutyl]amino}pyridine-2-carbonyl}amino-piperidine-(5R)-carboxylic acid (140 mg, 0.26 mmol, 1 eq), DIEA (40 mg, 0.31 mmol, 1.2 eq) and HATU (116.6 mg, 0.31 mmol, 1.2 eq) in DMF (1.5 mL) was added diimine hydrate hydrogen (25.08 mg, 0.512 mmol, 2 eq) at room temperature. The resulting mixture was stirred for 1 h at room temperature under nitrogen atmosphere. The residue was purified by reverse flash chromatography with the following conditions: column, C18 silica gel; mobile phase, MeCN in Water (0.1% T FA), 0% to 60% gradient in 20 min; UV 254 nm. This resulted in 1-tert-butoxylcarbonyl-(3S)—{N-(2-methylpropyl), N-{5-cyclopropyl-3-[3-(trans-methoxy-d3)cyclobutyl]amino}pyridine-2-carbonyl}amino-(5R)-(hydrazinecarbonyl)-piperidine (115 mg, 80%) as a yellow oil. LCMS (ESI) [M+H]⁺: 562.

### Synthesis of 1-tert-butoxycarbonyl-(3S)-{N-(2-methylpropyl), N-{5-cyclopropyl-3-[3-(trans-methoxy-d3) cyclobutyl]amino}pyridine-2-carbonyl}amino-(5R)-(1,3,4-oxadiazol-2-yl)-piperidine

To a stirred solution of 1-tert-butoxylcarbonyl-(3S)—{N-(2-methylpropyl), N-{5-cyclopropyl-3-[3-(trans-methoxy-d3)cyclobutyl]amino}pyridine-2-carbonyl}amino-(5R)-(hydrazinecarbonyl)-piperidine (150 mg, 0.27 mmol, 1 eq) in triethyl orthoformate (1.5 mL) was added p-Toluenesulfonic acid (4.6 mg, 0.027 mmol, 0.1 eq) at room temperature. The resulting mixture was stirred for 20 min at 125° C. under nitrogen atmosphere. The residue was purified by reverse flash chromatography with the following conditions: column, C18 silica gel; mobile phase, MeCN in Water (0.1% TFA), 0% to 50% gradient in 20 min; UV 254 nm. This resulted in 1-tert-butoxycarbonyl-(3S)—{N-(2-methylpropyl), N-{5-cyclopropyl-3-[3-(trans-methoxy-d3)cyclobutyl]amino}pyridine-2-carbonyl}amino-(5R)-(1,3,4-oxadiazol-2-yl)-piperidine (70 mg, 45%) as a white solid. LCMS (ESI) [M+H]⁺: 572.

### Synthesis of Compound 45

To a stirred solution of 1-tert-butoxycarbonyl-(3S)—{N-(2-methylpropyl), N-{5-cyclopropyl-3-[3-(trans-methoxy-d3)cyclobutyl]amino}pyridine-2-carbonyl}amino-(5R)-(1,3,4-oxadiazol-2-yl)-piperidine (65 mg, 0.11 mmol, 1 eq) in DCM (0.5 mL) was added TFA (0.5 mL) at room temperature. The resulting mixture was stirred for 30 min at room temperature under nitrogen atmosphere. The mixture was basified to pH 8 with saturated Na₂CO₃ (aq.). The crude product (65 mg) was purified by Prep-HPLC with the following conditions (Column: XBridge Shield RP18 OBD Column, 19*150 mm, 5 μm; Mobile Phase A: Water (10 mmol/L NH₄HCO₃), Mobile Phase B: MeOH—HPLC; Flow rate: 25 mL/min; Gradient: 45% B to 67% B in 7 min, 67% B; Wave Length: 220 nm; RT1 (min): 6.06; Number Of Runs: 0) to afford Compound 45 (29 mg, 54%) as a white solid. LCMS (ESI) [M+H]⁺: 472.20. ¹H NMR (400 MHz, DMSO-d6) δ 9.13 (s, 1H), 7.65 (d, J=16.3 Hz, 1H), 6.49 (s, 1H), 5.60 (d, J=64.0 Hz, 1H), 4.16-3.73 (m, 3H), 3.28-3.15 (m, 3H), 3.10-2.80 (m, 3H), 2.71-2.55 (m, 1H), 2.36-2.19 (m, 4H), 2.17-1.97 (m, 3H), 1.97-1.53 (m, 2H), 1.07-0.95 (m, 2H), 0.90 (d, J=6.6 Hz, 4H), 0.76 (s, 2H), 0.69-0.56 (m, 2H).

### Example 38. (3S)-{N-(2-methylpropyl), N-{2-cyclopropyl-3-[3-(trans-methoxy) cyclobutyl]amino}-pyrimidine-5-carbonyl}amino-(5R)-[(3S)-methylmorpholine-N-carbonyl]-piperidine (Compound 46)

### Synthesis of ethyl 2-cyclopropyl-4-oxo-5H-pyrimidine-5-carboxylate

To a stirred solution of cyclopropanecarboximidamide (1 g, 12 mmol, 1.0 eq) in ethanol (20 mL) was added 1,3-diethyl 2-(ethoxymethylidene)propanedioate (3.1 g, 14.3 mmol, 1.2 eq) at room temperature under nitrogen atmosphere. Then the resulting mixture was stirred overnight at room temperature under nitrogen atmosphere. The mixture was acidified to pH 6 with HCl (2 M). The aqueous layer was extracted with EtOAc (3×30 mL). The combined organic layers were dried over anhydrous Na₂SO₄. After filtration, the filtrate was concentrated under reduced pressure. The residue was purified by silica gel column chromatography, eluted with PE/EA (1:1) to afford ethyl 2-cyclopropyl-4-oxo-5H-pyrimidine-5-carboxylate (750 mg, 30%) as a white solid. LCMS (ESI) [M+H]⁺: 209.

### Synthesis of ethyl 4-chloro-2-cyclopropylpyrimidine-5-carboxylate

To a stirred solution of ethyl 2-cyclopropyl-4-oxo-5H-pyrimidine-5-carboxylate (750 mg, 3.6 mmol, 1.0 eq) in triethylamine (10 mL) was added phosphorus oxychloride (2761 mg, 18 mmol, 5.0 eq) at room temperature. The resulting mixture was stirred overnight at 100° C. under nitrogen atmosphere. The resulting mixture was extracted with EtOAc (30 mL). The combined organic layers were washed with water (3×30 mL), dried over anhydrous Na₂SO₄. After filtration, the filtrate was concentrated under reduced pressure and purified by reversed-phase flash chromatography with the following conditions: column, C18 silica gel; mobile phase, MeCN in Water (0.1% TFA), 0% to 60% gradient in 20 min; UV 254 nm. This resulted in ethyl 4-chloro-2-cyclopropylpyrimidine-5-carboxylate (300 mg, 37%) as a yellow oil. LCMS (ESI) [M+H]⁺: 227.

### Synthesis of ethyl 2-cyclopropyl-4-[3-(trans-methoxy)cyclobutyl]amino-pyrimidine-5-carboxylate

To a stirred solution of ethyl 4-chloro-2-cyclopropylpyrimidine-5-carboxylate (300 mg, 1.32 mmol, 1.0 eq) and DIEA (513.2 mg, 3.97 mmol, 3.0 eq) in NMP (5 mL) was added (1R,3R)-3-methoxycyclobutan-1-amine hydrochloride (218.5 mg, 1.59 mmol, 1.2 eq). The resulting mixture was stirred for 4 h at 140° C. under nitrogen atmosphere. After completion of reaction, the residue was purified by reversed-phase flash chromatography with the following conditions: column, C18 silica gel; mobile phase, MeCN in Water (0.1% TFA), 0% to 60% gradient in 20 min; UV 254 nm to afford ethyl 2-cyclopropyl-4-[3-(trans-methoxy)cyclobutyl]amino-pyrimidine-5-carboxylate (150 mg, 39%) as a yellow oil. LCMS (ESI) [M+H]⁺: 292.

### Synthesis of 2-cyclopropyl-4-[3-(trans-methoxy)cyclobutyl]amino-pyrimidine-5-carboxylic acid

To a stirred solution of ethyl 2-cyclopropyl-4-[3-(trans-methoxy)cyclobutyl]amino-pyrimidine-5-carboxylate (150 mg, 0.51 mmol, 1.0 eq) and H₂O (1 mL) in THE (2 mL) was added lithium hydrate (108 mg, 2.57 mmol, 5.0 eq) at room temperature under nitrogen atmosphere. The resulting mixture was stirred for 2 h at room temperature under nitrogen atmosphere. After completion of reaction, the mixture was acidified to pH 6 with citric acid and purified by reversed-phase flash chromatography with the following conditions: column, C18 silica gel; mobile phase, MeCN in Water (0.1% FA), 0% to 60% gradient in 20 min; UV 254 nm to afford 2-cyclopropyl-4-[3-(trans-methoxy)cyclobutyl]amino-pyrimidine-5-carboxylic acid (120 mg, 89%) as a yellow oil. LCMS (ESI) [M+H]⁺: 264. ¹H NMR (300 MHz, DMSO-d6) δ 13.24 (s, 1H), 8.56 (s, 1H), 8.44 (s, 1H), 4.66-4.47 (m, 1H), 4.06-3.92 (m, 1H), 3.16 (s, 3H), 2.41-2.27 (m, 2H), 2.28-2.11 (m, 2H), 2.00 (m, 1H), 1.08-0.93 (m, 4H).

### Synthesis of methyl 1-tert-butoxy/carbonyl-(3S)-{N-(2-methylpropyl), N-{2-cyclopropyl-4-[3-(trans-methoxy) cyclobutyl]amino}pyrimidine-5-carbonyl}amino-piperidine-(5R)-carboxylate

To a stirred solution of 2-cyclopropyl-4-[3-(trans-methoxy)cyclobutyl]amino-pyrimidine-5-carboxylic acid (120 mg, 0.45 mmol, 1.0 eq), DIEA (17.6 mg, 0.14 mmol, 3.0 eq) and HATU (207.9 mg, 0.55 mmol, 1.2 eq) in DMF (4 mL) was added Compound A (143.3 mg, 0.45 mmol, 1.0 eq) at room temperature under nitrogen atmosphere. The resulting mixture was stirred for 1 h at room temperature under nitrogen atmosphere. After completion of reaction, the residue was purified by reversed-phase flash chromatography with the following conditions: column, C18 silica gel; mobile phase, MeCN in Water (0.1% TFA), 0% to 60% gradient in 10 min; UV 254 nm to afford methyl 1-tert-butoxylcarbonyl-(3S)—{N-(2-methylpropyl), N-{2-cyclopropyl-4-[3-(trans-methoxy)cyclobutyl]amino}pyrimidine-5-carbonyl}amino-piperidine-(5R)-carboxylate (130 mg, 51%) as a yellow solid. LCMS (ESI) [M+H]⁺: 560.

### Synthesis of 1-tert-butoxylcarbonyl-(3S)-{N-(2-methylpropyl), N-{2-cyclopropyl-4-[3-(trans-methoxy) cyclobutyl]amino}pyrimidine-5-carbonyl}amino-piperidine-(5R)-carboxylic acid

To a stirred solution of methyl 1-tert-butoxylcarbonyl-(3S)—{N-(2-methylpropyl), N-{2-cyclopropyl-4-[3-(trans-methoxy)cyclobutyl]amino}pyrimidine-5-carbonyl}amino-piperidine-(5R)-carboxylate (130 mg, 0.23 mmol, 1.0 eq) and H₂O (1 mL) in THF (2 mL) was added lithium hydrate (48.7 mg, 1.16 mmol, 5.0 eq) at room temperature under nitrogen atmosphere. The resulting mixture was stirred for 2 h at room temperature under nitrogen atmosphere. After completion of reaction, the mixture was acidified to pH 6 with citric acid and purified by reversed-phase flash chromatography with the following conditions: column, C18 silica gel; mobile phase, MeCN in Water (0.1% TFA), 0% to 60% gradient in 20 min; UV 254 nm to afford 1-tert-butoxylcarbonyl-(3S)—{N-(2-methylpropyl), N-{2-cyclopropyl-4-[3-(trans-methoxy)cyclobutyl]amino}pyrimidine-5-carbonyl}amino-piperidine-(5R)-carboxylic acid (100 mg, 79%) as a yellow oil. LCMS (ESI) [M+H]⁺: 545. ¹H NMR (400 MHz, DMSO-d6) δ 12.56 (s, 1H), 7.92 (s, 1H), 6.98 (s, 1H), 4.59-4.45 (m, 1H), 4.13-3.89 (m, 3H), 3.87-3.46 (m, 2H), 3.23-3.09 (m, 5H), 2.80-2.57 (m, 1H), 2.39-2.12 (m, 6H), 2.01-1.88 (m, 3H), 1.45-1.21 (m, 9H), 1.00-0.73 (m, 10H).

### Synthesis of 1-tert-butoxycarbonyl-(3S)-{N-(2-methylpropyl), N-{2-tert-butyl-4-[3-(trans-methoxy) cyclobutyl]amino}pyrimidine-5-carbonyl}amino-(5R)-[(3S)-methylmorpholine-N-carbonyl]-piperidine

To a stirred solution of 1-tert-butoxylcarbonyl-(3S)—{N-(2-methylpropyl), N-{2-cyclopropyl-4-[3-(trans-methoxy)cyclobutyl]amino}pyrimidine-5-carbonyl}amino-piperidine-(5R)-carboxylic acid (100 mg, 0.18 mmol, 1.0 eq), DIEA (71 mg, 0.55 mmol, 3.0 eq) and HATU (84 mg, 0.22 mmol, 1.2 eq) in DMF was added (3 S)-3-methylmorpholine (22.2 mg, 0.22 mmol, 1.2 eq) at room temperature under nitrogen atmosphere. The resulting mixture was stirred for 1 h at room temperature under nitrogen atmosphere. After completion of reaction, the residue was purified by reversed-phase flash chromatography with the following conditions: column, C18 silica gel; mobile phase, MeCN in Water (0.1% TFA), 0% to 60% gradient in 10 min; UV 254 nm to afford 1-tert-butoxycarbonyl-(3S)—{N-(2-methylpropyl), N-{2-tert-butyl-4-[3-(trans-methoxy)cyclobutyl]amino}pyrimidine-5-carbonyl}amino-(5R)-[(3S)-methylmorpholine-N-carbonyl]-piperidine (95 mg, 82%) as a yellow solid. LCMS (ESI) [M+H]⁺: 629. ¹H NMR (400 MHz, Chloroform-d) δ 8.00 (s, 1H), 6.81 (d, J=37.0 Hz, 1H), 4.66-4.50 (m, 1H), 4.07-3.98 (m, 2H), 3.98-3.84 (m, 2H), 3.80-3.68 (m, 1H), 3.68-3.46 (m, 3H), 3.26 (s, 3H), 3.21-2.89 (m, 3H), 2.85-2.52 (m, 3H), 2.51-2.25 (m, 6H), 2.24-2.12 (m, 2H), 2.02-1.84 (m, 2H), 1.51-1.34 (m, 9H), 1.30-1.21 (m, 3H) 1.18-0.83 (m, 10H).

### Synthesis of Compound 46

To a stirred solution of 1-tert-butoxycarbonyl-(3S)—{N-(2-methylpropyl), N-{2-tert-butyl-4-[3-(trans-methoxy)cyclobutyl]amino}pyrimidine-5-carbonyl}amino-(5R)-[(3 S)-methylmorpholine-N-carbonyl]-piperidine (95 mg, 0.11 mmol, 1.0 eq) in DCM (2 mL) was added TFA (0.5 mL). The resulting mixture was stirred for 20 min at room temperature under nitrogen atmosphere. The mixture was basified to pH 9 with saturated Na₂CO₃ (aq.). The aqueous layer was extracted with EtOAc (3×10 mL). The combined organic layers were dried over anhydrous Na₂SO₄. After filtration, the filtrate was concentrated under reduced pressure. The crude product was purified by Prep-HPLC with the following conditions to afford Compound 46 (37 mg, 46%) as a white solid. LCMS (ESI) [M+H]⁺: 529.30. ¹H NMR (400 MHz, DMSO-d6) δ 7.89 (s, 1H), 6.91 (s, 1H), 4.58-4.43 (m, 1H), 4.41-4.28 (m, 1H), 4.05-3.87 (m, 2H), 3.85-3.74 (m, 1H), 3.61 (d, J=11.5 Hz, 2H), 3.48-3.37 (m, 1H), 3.28-3.18 (m, 1H), 3.17-3.05 (m, 5H), 2.92-2.53 (m, 5H), 2.39-2.13 (m, 6H), 2.07-1.86 (m, 3H), 1.87-1.72 (m, 1H), 1.24-1.03 (m, 3H), 0.98-0.89 (m, 4H), 0.88-0.70 (m, 6H).

### Example 39. Inhibitory Effects of Exemplary Compounds of the Disclosure

The exemplary compounds of the disclosure were tested for their inhibitory effects on human renin activity, using a commercially available renin assay kit (SensoLyte® 520 Renin Assay Kit Fluorimetric), following the manufacture's manual.

Briefly, human recombinant renin (from the kit) was incubated with the compound of the disclosure or buffer for 30 min at 37° C. before substrate addition. Fluorescence intensity was measured at 530 nm after an incubation of 15 min at 37° C. Data was analyzed by Prism and fitted into a sigmodal curve for IC₅₀ calculation.

The results were summarized in Table 2.

TABLE 2
Inhibitory effect of compounds of the
disclosure on human renin activity

| Compound No. | Example No. | IC₅₀ (nM) |
|---|---|---|
| 10 | 2 | 1.8 |
| 11 | 3 | 1.2 |
| 12 | 4 | 1.6 |
| 13 | 5 | 3.8 |
| 14 | 6 | 1.2 |
| 15 | 7 | 1.0 |
| 16 | 8 | 1.6 |
| 17 | 9 | 1.5 |
| 18 | 10 | 1.2 |
| 19 | 11 | 1.5 |
| 20 | 12 | 1.9 |
| 21 | 13 | 1.4 |
| 22 | 14 | 1.3 |
| 23 | 15 | 1.2 |
| 24 | 16 | 1.0 |
| 25 | 17 | <10 |
| 26 | 18 | <20 |
| 27 | 19 | <250 |
| 28 | 20 | <10 |
| 29 | 21 | <10 |
| 30 | 22 | <10 |
| 31 | 23 | <10 |
| 32 | 24 | <10 |
| 33 | 25 | <250 |
| 34 | 26 | <10 |
| 35 | 27 | <20 |
| 36 | 28 | <50 |
| 37 | 29 | <20 |
| 38 | 30 | <50 |
| 39 | 31 | <10 |
| 40 | 32 | <10 |
| 41 | 33 | <10 |
| 42 | 34 | <30 |
| 43 | 35 | <10 |
| 44 | 36 | <10 |
| 45 | 37 | <10 |
| 46 | 38 | <250 |

The invention has been illustrated by the above descriptions and examples. The examples are not intended to be limiting in any way. As used throughout, ranges are used as shorthand for describing each and every value that is within the range. Any value within the range can be selected as the terminus of the range.

What is claimed is:

1. A compound of formula I,
or a geometric isomer, a pharmaceutically acceptable isotopic isomer, salt, prodrug or solvate thereof,
wherein R₁ and R₂ are independently a hydrogen atom, a deuterium atom, a halogen atom, a hydroxyl group, a carboxyl group, a cyano group, an optionally substituted C₁₋₆ alkyl group, an optionally substituted C₂₋₆ alkenyl group, an optionally substituted C₂₋₆ alkynyl group, an optionally substituted C₁₋₃ alkylthio group, an optionally substituted C₁₋₃ alkylsulfinyl group, an optionally substituted C₁₋₃ alkylsulfonyl group, an optionally substituted C₁₋₆ alkoxy group, an optionally substituted C₁₋₆ alkoxyC₁₋₆ alkyl group, an optionally substituted C₁₋₃ alkyl C₁₋₆ alkoxy group, an optionally substituted C₃₋₆ cycloalkyl group, an optionally substitute C₁₋₃alkyl C₃₋₆ cycloalkyl group, an optionally substituted amino group, an optionally substituted C₁₋₃ alkyl amino group, an optionally substituted mercapto group, an optionally substituted aminocarbonyl group, an optionally substituted carbonyl group, an optionally substituted C₁₋₆ alkyl carbonyl group, or an optionally substituted C₁₋₃ alkoxycarbonyl group,
n is 0, 1, 2 or 3,
X is a methylene group, an oxygen atom, an amine group, a sulfinyl group or a sulfonyl group, Ring A is an optionally substituted 5- or 6-membered heterocycle that contains one or more N, O, S, SO and SO₂,
R₃ is a hydrogen atom, a deuterium atom, a halogen atom, a hydroxyl group, a carboxyl group, an optionally substituted C₁₋₆ alkyl group, an optionally substituted C₃₋₆ cycloalkyl group, an optionally substituted C₂₋₆ alkenyl group, an optionally substituted C₂₋₆ alkynyl group, an optionally substituted C₁₋₆ alkoxy group, an optionally substituted amino group, an optionally substituted mercapto group, an optionally substituted aminocarbonyl group, an optionally substituted C₁₋₆ alkylcarbonyl group, an optionally substituted C₁₋₆ alkoxyC₁₋₆ alkyl group, or an optionally substituted C₁₋₃ alkoxycarbonyl group,
R₄ is a hydrogen atom, a halogen atom, a deuterium atom, a hydroxyl group, a cyano group, an optionally substituted amino group, an optionally substituted C₁₋₆ alkyl group, an optionally substituted C₁₋₆ haloalkyl group, an optionally substituted C₂₋₆ alkenyl group, an optionally substituted C₂₋₆ alkynyl group, an optionally substituted mercapto group, an optionally substituted C₁₋₆ alkylsulfinyl group, an optionally substituted C₁₋₃ alkylsulfonyl group, an optionally substituted C₁₋₆ alkoxy group, an optionally substituted C₁₋₆ haloalkoxy group, an optionally substituted C₃₋₆ cycloalkyl group, an optionally substituted aminocarbonyl group, an optionally substituted C₁₋₆ alkylcarbonyl group, an optionally substituted C₁₋₆ haloalkylcarbonyl group, an optionally substituted C₁₋₃ alkoxycarbonyl group, an optionally substituted C₁₋₃ haloalkoxycarbonyl group, an optionally substituted C₁₋₆ alkoxyC₁₋₆ alkyl group, an optionally substituted C₁₋₆ haloalkoxyC₁₋₆ alkyl group, a 4- to 6-membered heterocycle substituted formyl group wherein the 4 to 6-membered heterocycle may contain one or more N, O, S, SO, and SO₂ and may be optionally substituted, an optionally substituted 3 to 6-membered cyclic hydrocarbon group, an optionally substituted 5- or 6-membered heterocycle wherein the heterocycle may contain one or more N, O, S, SO, and SO₂, or an optionally substituted amide group, and
R₅ is a hydrogen atom, a deuterium atom, a halogen atom, a hydroxyl group, a cyano group, a mercapto group, an optionally substituted C₁₋₃ alkylsulfinyl group, an optionally substituted C₁₋₃ alkylsulfonyl group, an optionally substituted C₁₋₃ haloalkylsulfonyl group, an optionally substituted amino group, an optionally substituted C₁₋₆ alkyl group, an optionally substituted C₁₋₆ haloalkyl group, an optionally substituted C₂₋₆ alkenyl group, an optionally substituted C₂₋₆ alkynyl group, an optionally substituted C₁₋₆ alkoxy group, an optionally substituted C₁₋₆haloalkoxy group, an optionally substituted C₃₋₆ cycloalkyl group, an optionally substituted C₃₋₆ halocycloalkyl group, an optionally substituted C₁₋₃ alkylthio group, an optionally substituted C₁₋₃ alkylsulfinyl group, an optionally substituted C₁₋₃ haloalkylsulfinyl group, an optionally substituted C₃₋₄ cycloalkyl group, an optionally substituted aminocarbonyl group, an optionally substituted C₁₋₆ alkylcarbonyl group, an optionally substituted C₁₋₆ alkoxyC₁₋₆ alkyl group, an optionally substituted C₁₋₆ haloalkoxyC₁₋₆ alkyl group, an optionally substituted C₁₋₃ alkoxycarbonyl group, or an optionally substituted C₁₋₃ haloalkoxycarbonyl group.

2. The compound of claim 1, wherein ring A is an optionally substituted 5- or 6-membered unsaturated heterocycle containing one or two atoms selected from N, O and S.

3. The compound of claim 2, wherein ring A is one of the following:
wherein R₁′, R₂′, and R₃′ are independently a hydrogen atom, a deuterium atom, a halogen atom, a cyano group, a hydroxyl group, an optionally substituted C₁₋₆ alkyl group, an optionally substituted C₁₋₆ haloalkyl group, an optionally substituted C₂₋₆ alkenyl group, an optionally substituted C₂₋₆ haloalkenyl group, an optionally substituted C₂₋₆ alkynyl group, an optionally substituted C₂₋₆ haloalkynyl group, an optionally substituted C₃₋₆ cycloalkyl group, an optionally substituted C₁₋₆ alkylsulfinyl group, an optionally substituted C₁₋₆ haloalkylsulfinyl group, an optionally substituted C₁₋₆ alkylsulfonyl group, an optionally substituted C₁₋₆ haloalkylsulfonyl group, an optionally substituted C₁₋₆ alkoxy group, an optionally substituted C₁₋₆ haloalkoxy group, an optionally substituted amino group, an optionally substituted C₁₋₆ alkylcarbonyl group, an optionally substituted C₁₋₆ haloalkylcarbonyl group, an optionally substituted C₁₋₃ alkoxycarbonyl group, an optionally substituted C₁₋₃ haloalkoxycarbonyl group, an optionally substituted C₁₋₆ alkoxyC₁₋₆ alkyl group, an optionally substituted C₁₋₆ haloalkoxyC₁₋₆ alkyl group, or an optionally substituted 5 or 6-membered heterocycle containing one or more N, O, S, SO, and SO₂.

4. The compound of claim 1, wherein R₁ and R₂ are independently a hydrogen atom, a halogen atom, a hydroxyl group, a C₁₋₆ alkyl group, a C₁₋₆ haloalkyl group, a C₃₋₆ cycloalkyl group which is unsubstituted or substituted with one or more selected from the group consisting of a halogen atom, a C₁₋₃ alkyl group and a C₁₋₃ haloalkyl group, a C₁₋₆ alkoxy group which is unsubstituted or substituted with one or more selected from the group consisting of a halogen atom, a C₁₋₃ alkyl group and a C₁₋₃ haloalkyl group, an amino group which is unsubstituted or substituted with one or more selected from the group consisting of a halogen atom, a C₁₋₃ alkyl group and a C₁₋₃ haloalkyl group, an aminocarbonyl group which is unsubstituted or substituted with one or more selected from the group consisting of a halogen atom, a C₁₋₃ alkyl group and a C₁₋₃ haloalkyl group, a C₁₋₆ alkylcarbonyl group, a C₁₋₆ haloalkylcarbonyl group, a C₁₋₆ alkoxyC₁₋₆ alkyl group, a C₁₋₆ haloalkoxyC₁₋₆ alkyl group, a C₁₋₃ alkoxycarbonyl group or a C₁₋₃ halolkoxycarbonyl group.

5. The compound of claim 3, wherein R₁′, R₂′, and R₃′ are independently a hydrogen atom, a halogen group, a hydroxyl group, a C₁₋₆ alkyl group, a C₁₋₆ haloalkyl group, a C₃₋₆ cycloalkyl group which is unsubstituted or substituted with one or more selected from the group consisting of a halogen atom, a C₁₋₃ alkyl group and a C₁₋₃ haloalkyl group, a C₁₋₆ alkoxy group, a C₁₋₆ haloalkoxy group, an amino group which is unsubstituted or substituted with one or more selected from the group consisting of a halogen atom, a C₁₋₃ alkyl group and a C₁₋₃ haloalkyl group, a C₁₋₆ alkylcarbonyl group, a C₁₋₆ haloalkylcarbonyl group, a C₁₋₃ alkoxycarbonyl group, a C₁₋₃ haloalkoxycarbonyl group, a C₁₋₆ alkoxyC₁₋₆ alkyl group, or a C₁₋₆ haloalkoxyC₁₋₆ alkyl group.

6. The compound of claim 1, wherein R₃ is a hydrogen atom, a deuterium atom, a halogen atom, a C₁₋₆ alkyl group which is unsubstituted or substituted with one or more selected from the group consisting of a halogen atom, a C₁₋₃ alkyl group and a C₁₋₃ haloalkyl group, a C₃₋₆ cycloalkyl group which is unsubstituted or substituted with one or more selected from the group consisting of a halogen atom, a C₁₋₃ alkyl group and a C₁₋₃ haloalkyl group, a C₁₋₆ alkoxy group which is unsubstituted or substituted with one or more selected from the group consisting of a halogen atom, a C₁₋₃ alkyl group and a C₁₋₃ haloalkyl group, an amino group which is unsubstituted or substituted with one or more selected from the group consisting of a halogen atom, a C₁₋₃ alkyl group and a C₁₋₃ haloalkyl group, an aminocarbonyl group, a C₁₋₆ alkylcarbonyl group, a C₁₋₆ alkoxyC₁₋₆ alkyl group, or a C₁₋₃ alkoxycarbonyl group.

7. The compound of claim 1, wherein R₄ is a 3 to 6-membered cyclic hydrocarbon group which is unsubstituted or substituted with one or more selected from the group consisting of a halogen atom, a C₁₋₃ alkyl group and a C₁₋₃ haloalkyl group; a 5- or 6-membered unsaturated heterocycle which contains one or more selected from the group consisting of N, O, S, SO and SO₂ and is unsubstituted or substituted by one or more selected from the group consisting of a halogen atom, a hydroxyl group, a C₁₋₆ alkyl group, a C₁₋₆ haloalkyl group, a C₃₋₆ cycloalkyl group, a C₃₋₆ halocycloalkyl group, a hydroxyl C₁₋₆ alkyl group, a hydroxyl C₃₋₆ cycloalkyl group, a C₁₋₆ alkylcarbonyl group, a C₃₋₆ halocycloalkylcarbonyl group, a C₁₋₆ haloalkyl group, a C₃₋₆ halocycloalkyl group, a C₁₋₆ haloalkylcarbonyl group, a C₃₋₆ halocycloalkylcarbonyl group, a C₁₋₃ alkoxyC₁₋₃ alkyl group, a C₁₋₆ alkyl amino group, a 3- or 6-membered heterocycle which contains one or more selected from the group consisting of N, O, S, SO and SO₂ and is unsubstituted or substituted by a halogen atom, a C₁₋₃ alkyl group and a C₁₋₃ haloalkyl group, and a 3 to 6-membered cyclic hydrocarbon group which is unsubstituted or substituted with one or more selected from the group consisting of a halogen atom, a C₁₋₃ alkyl group and a C₁₋₃ haloalkyl group; a 4- to 6-membered heterocycle substituted formyl group wherein the heterocycle contains one or more selected from the group consisting of N, O, S, SO, and SO₂ and is unsubstituted or substituted by one or more selected from the group consisting of a halogen atom, a hydroxyl group, a C₁₋₆ alkyl group, a C₁₋₆ haloalkyl group, a C₃₋₆ cycloalkyl group, a C₃₋₆ halocycloalkyl group, a C₁₋₆ alkoxy group, a C₁₋₆ haloalkoxy group, a hydroxyl C₁₋₆ alkyl group, a hydroxyl C₃₋₆ cycloalkyl group, a C₁₋₆ alkylcarbonyl group, a C₃₋₆ halocycloalkylcarbonyl group, a C₁₋₆ haloalkyl group, a C₃₋₆ halocycloalkyl group, a C₁₋₆ haloalkylcarbonyl group, a C₃₋₆ halocycloalkylcarbonyl group, a C₁₋₃ alkoxyC₁₋₃ alkyl group, and a C₁₋₆ alkyl amino group; or a hydroxyl C₁₋₆ alkyl group.

8. The compound of claim 1, wherein R₅ is a hydrogen atom, a halogen atom, a hydroxyl group, a C₁₋₆ alkoxy group, a C₁₋₆ haloalkoxy group, an amino group which is unsubstituted or substituted with one or more selected from the group consisting of a halogen atom, a C₁₋₃ alkyl group and a C₁₋₃ haloalkyl group, a C₁₋₆ alkyl group, a C₁₋₆ haloalkyl group, a C₃₋₆ cycloalkyl group which is unsubstituted or substituted with one or more selected from the group consisting of a halogen atom, a C₁₋₃ alkyl group and a C₁₋₃ haloalkyl group, a C₁₋₃ alkylsulfinyl group, a C₁₋₃ haloalkylsulfinyl group, a C₁₋₃ alkylsulfonyl group, a C₁₋₃ haloalkylsulfonyl group, an aminocarbonyl group which is unsubstituted or substituted with one or more selected from the group consisting of a halogen atom, a C₁₋₃ alkyl group and a C₁₋₃ haloalkyl group, a C₁₋₆ alkylcarbonyl group, a C₁₋₆ alkoxyC₁₋₆ alkyl group, a C₁₋₆ haloalkoxyC₁₋₆ alkyl group, a C₁₋₃ alkoxycarbonyl group, or a C₁₋₃ haloalkoxycarbonyl group.

9. A compound of formula II,
or a geometric isomer, a pharmaceutically acceptable isotopic isomer, salt, prodrug or solvate thereof,
wherein R₁, R₂, R₃, R₅, X, n, and ring A are defined as in claim 1, and
wherein R₆ and R₇ are independently a hydrogen atom, a deuterium atom, a halogen atom, a cyano group, a hydroxyl group, an optionally substituted C₁₋₃ alkyl group, an optionally substituted C₃₋₆ cycloalkyl group, or an optionally substituted C₁₋₃ alkoxy group.

10. The compound of claim 9, wherein R₆ and R₇ are independently a hydrogen atom, a deuterium atom, a halogen atom, a cyano group, a hydroxyl group, a C₁₋₃ alkyl group, a C₁₋₃ haloalkyl group, a C₃₋₆ cycloalkyl group, a C₃₋₆halocycloalkyl group, a C₁₋₃ alkoxy group, or a C₁₋₃ haloalkoxy group.

11. The compound of claim 1, selected from the group consisting of:
(3S)—[N-(2-methylpropyl), N-(5-cyclopropyl-3-cyclopropylamino)pyridine-2-carbonyl]amino-(5R)-[(3S)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(3-fluoroazetidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(3,3-difluoropyrrolidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(4,4-difluoropiperidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(4-trifluoromethoxypiperidine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-[(3R)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-[(3S)-methylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(2,2-dimethylmorpholine-N-carbonyl)-piperidine,
(3S)—{N-(2-methylpropyl), N-{2-tert-butyl-3-[3-(trans-methoxy)cyclobutyl]amino}pyrimidine-5-carbonyl}amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(morpholine-N-carbonyl)-piperidine,
(3S)—{N-(2-methylpropyl), N-{5-cyclopropyl-3-[3-(cis-methoxy)cyclobutyl]amino}pyridine-2-carbonyl}amino-(5R)-(morpholine-N-carbonyl)-piperidine,
(3S)—[N-(2-methylpropyl), N-(5-cyclopropyl-3-cyclobutylamino)pyridine-2-carbonyl]amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-(5R)-(5-methyl-1,3,4-oxadiazol-2-yl)-piperidine,
(3S)—{N-(2-methylpropyl), N-{5-cyclopropyl-3-[3-(trans-methoxy)cyclobutyl]amino}pyridine-2-carbonyl}amino-(5R)-(5-cyclopropyl-1,3,4-oxadiazol-2-yl)-piperidine,
(3S)—{N-(2-methylpropyl), N-{5-cyclopropyl-3-[3-(trans-methoxy)cyclobutyl]amino}pyridine-2-carbonyl}amino-(5R)-(3,3-diflurorocyclobutyl-1,3,4-oxadiazol-2-yl)-piperidine,
(3S)—[N-(2-methylpropyl), N-(5-cyclopropyl-3-cyclopropylamino)pyridine-2-carbonyl]amino-(5R)-(morpholine-N-carbonyl)-piperidine,
(3S)—{N-(2-methylpropyl), N-{5-cyclopropyl-3-[3-(trans-methoxy)cyclobutyl]amino}pyridine-2-carbonyl}amino-piperidine,
(3S)—{N-(2-methylpropyl), N-{5-cyclopropyl-3-[3-(trans-methoxy)cyclobutyl]amino}pyridine-2-carbonyl}amino-(5R)-[(3S)-ethylmorpholine-N-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-(5-methyl-1,2,4-oxadiazol-3-yl)-piperidine,
(3S)—{N-(2-methylpropyl), N-{5-cyclopropyl-3-[3-(trans-methoxy)cyclobutyl]amino}pyridine-2-carbonyl}amino-(5R)-(3-methyl-1,2,4-oxadiazol-5-yl)-piperidine,
(3S)—[N-(2-methylpropyl), N-(5-cyclopropyl-3-(oxetan-3-ylamino)pyridine-2-carbonyl]amino-(5R)-(morpholine-N-carbonyl)-piperidine,
(3S)—{N-(2-methylpropyl), N-[3-(3-trans-methoxy)cyclobutyl]amino-pyridine-2-carbonyl}amino-(5R)-(5-cyclopropyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(5-cyclopropyl-1,3,4-oxadiazol-2-yl)-piperidine,
(3S)—{N-(2-methylpropyl), N-{6-cyclopropyl-4-[3-(trans-methoxy)cyclobutyl]amino}pyridazine-3-carbonyl}amino-(5R)-[(3S)-3-methylmorpholine-4-carbonyl]-piperidine,
(3S)—{N-(2-methylpropyl), N-{5-cyclopropyl-3-[3-(trans-methoxy)cyclobutyl]amino}pyridine-2-carbonyl}amino-(5R)-[5-(oxetan-3-yl)-1,3,4-oxadiazol-2-yl]-piperidine,
(3S)—{N-(2-methylpropyl), N-{5-cyclopropyl-3-[3-(trans-methoxy)cyclobutyl]amino}pyridine-2-carbonyl}amino-(5R)-hydroxymethyl-piperidine,
(3S)—{N-(2-methylpropyl), N-[5-cyclopropyl-3-(oxetan-3-yl)amino]pyridine-2-carbonyl}amino-piperidine,
(3S)—{N-(2-methylpropyl), N-[5-cyclopropyl-3-(oxetan-3-yl)amino]pyridine-2-carbonyl}amino-(5R)-(5-methyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(3-methyl-1,2,4-oxadiazol-5-yl)-piperidine,
(3S)—{N-(2-methylpropyl), N-{5-cyclopropyl-3-[3-(trans-methoxy)cyclobutyl]amino}pyridine-2-carbonyl}amino-(5R)-(1,2,4-oxadiazol-3-yl)-piperidine,
(3S)—{N-(2-methylpropyl), N-{5-cyclopropyl-3-[3-(trans-methoxy)cyclobutyl]amino}pyridine-2-carbonyl}amino-(5R)-(1,3,4-oxadiazol-2-yl)-piperidine,
(3S)—{N-(2-cyclopropylmethyl, N-{5-cyclopropyl-3-[3-(trans-methoxy)cyclobutyl]amino}-pyridine-2-carbonyl}amino-(5R)-(5-methyl-1,3,4-oxadiazol-2-yl)-piperidine,
(3S)—{N-(2-methylpropyl), N-[5-cyclopropyl-3-(oxetan-3-yl)amino]pyridine-2-carbonyl}amino-(5R)-(1,2,4-oxadiazol-3-yl)-piperidine,
(3S)—{N-(2-methylpropyl), N-[5-trifluoromethyl-3-(oxetan-3-yl)amino]pyridine-2-carbonyl}amino-(5R)-(morpholine-N-carbonyl)-piperidine,
(3S)—{N-cyclopropylmethyl, N-{5-cyclopropyl-3-[3-(trans-methoxy)cyclobutyl]amino}pyridine-2-carbonyl}amino-(5R)-(1,3,4-oxadiazol-2-yl)-piperidine,
(3S)—{N-(2-methylpropyl), N-{5-cyclopropyl-3-[3-(trans-methoxy-d3)cyclobutyl]amino}pyridine-2-carbonyl}amino-(5R)-(1,3,4-oxadiazol-2-yl)-piperidine, and
(3S)—{N-(2-methylpropyl), N-{2-cyclopropyl-3-[3-(trans-methoxy)cyclobutyl]amino}pyrimidine-5-carbonyl}amino-(5R)-[(3S)-methylmorpholine-N-carbonyl]-piperidine.

12. A pharmaceutical composition comprising an effective amount of a compound, or a geometric isomer, a pharmaceutically acceptable isotopic isomer, salt, prodrug or solvate thereof, of claim 1, and a pharmaceutically acceptable carrier.

13. A method for inhibiting renin activity in a subject in need thereof, comprising administering to the subject a pharmaceutical composition of claim 12.

14. A method for treating a disease associated with renin activity in a subject in need thereof, comprising administering to the subject a pharmaceutical composition of claim 12.

15. The method of claim 14, wherein the disease is hypertension, cardiovascular disease, diabetic kidney disease, or heart failure.

16. The method of claim 15, wherein the disease is hypertension.
